(19) United States
(12) Patent Application Publication
(10) Pub. No.: US 2024/0294548 A1
(43) Pub. Date: Sep. 5, 2024

(54) TRICYCLIC SULTAMS AND SULFAMIDES AS ANTITUMOR AGENTS

(71) Applicant: PELOTON THERAPEUTICS INC., Rahway, NJ (US)

(72) Inventors: James P. Rizzi, Aurora, CO (US); Paul Wehn, Dallas, TX (US)

(21) Appl. No.: 18/681,173

(22) PCT Filed: Sep. 14, 2022
(86) PCT No.: PCT/US22/43437

§ 371 Date: Feb. 5, 2024

Related U.S. Application Data
(60) Provisional application No. 63/254,566, filed on Oct. 12, 2021.

(51) Int. Cl. C07D 513/04; A61K 31/542; A61K 31/549

(52) U.S. Cl. CPC C07D 513/04; A61K 31/542; A61K 31/549

(57) ABSTRACT

Compounds of formula I, that modulate DCAF15 and induce RBM39 protein degradation, pharmaceutical compositions containing these compounds, and methods of using these compounds for treating diseases associated with RBM39 protein activity and DCAF15 modulation are described herein.

### BACKGROUND OF THE INVENTION

The frequent observation of aberrant pre-mRNA splicing in many cancers may offer opportunities for the development of novel therapeutic agents. For example, RBM39 protein is associated with core components of the spliceosome. Loss or reduction of amount of RBM39 protein can alter the frequency of alternative splicing events resulting in exon skipping and intron retention. Such events may trigger selective lethality in cancer cells reliant on altered splicing or induce expression of splicing-derived neoantigens that can be exploited for therapy. For example, it was found that anticancer sulfonamide, indisulam, targets pre-RNA splicing by inducing RBM39 degradation via recruitment of DCAF15 (See T. Han, et al., Anticancer sulfonamides target splicing by inducing RBM39 degradation via recruitment to DCAF15”, Science 356, eeal. 3755, (2017).

RBM39 protein is required for acute myeloid leukemia (AML) maintenance through misspicing of HOXA9 target genes and it has been found that RBM39 loss alters splicing of mRNAs essential for AML cell growth (See E. Wang et al., “Targeting an RNA-Binding Protein network in Acute Myeloid Leukemia”, Cancer Cell 35, 369-382, (2019) and D. Hsiehchen, et al., “Biomarkers for RBM39 degradation in acute myeloid leukemia”, Springer Nature, February 2020)). It is also believed that compounds able to degrade RBM39 may be effective in treating cancers such as colon, EZH2 mutant limphoma, and melanoma.

### SUMMARY OF THE INVENTION

The present invention relates to novel compounds, to pharmaceutical compositions comprising the compounds, to a process for making the compounds and to the use of the compounds in therapy. More particularly, it relates to certain tricyclic sulfamide and sultam derivatives useful in potential disease treatment via proteasomal degradation mechanism by modulating E3 ubiquitin ligase DCAF15 and recruiting neosubstrates such as RBM39 for degradation.

The invention identifies a class of tricyclic sulfamide and sultam derivatives which are capable of mediating the selective degradation of RBM39, an RNA binding protein associated with core components of the spliceosome. Loss or diminished amounts of RBM39 protein can alter the frequency of alternative splicing events resulting in exon skipping and intron retention. Such events may trigger selective lethality in cancer cells reliant on altered splicing or induce expression of splicing-derived neoantigens that can be exploited for therapy. In addition to RBM39 degradation, the disclosed compounds have superior binding affinities to the E3 ligase DCAF15. Small molecule binding to DCAF15 enables proximilization of RBM39 to the cullin ring ligase system for sequential ubiquitylation and proteasomal degradation of RBM39. It may be possible to harness the improved affinity of the disclosed compounds towards DCAF15 for novel applications such as in bifunctional degradation or in the construction of libraries to prospect for molecular glues capable of mediating the degradation of neo-substrates.

In one aspect, the present invention provides a compound of Formula I:

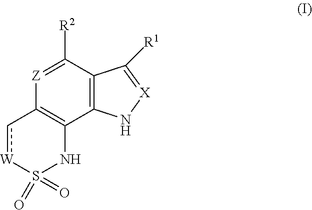

or a pharmaceutically acceptable salt thereof.

In another aspect, the present disclosure provides a pharmaceutical composition comprising a compound of Formula I or a pharmaceutically acceptable salt thereof, and a pharmaceutically acceptable diluent or carrier.

In another aspect, the present disclosure provides a method of inducing degradation of RBM39 protein, comprising contacting RBM39 protein with an effective amount of a compound described herein, such as a compound of Formula I. The contacting may comprise contacting a cell that expresses RBM39 protein and E3 ligase DCAF15. The contacting may take place in vivo or in vitro.

In another aspect, the present disclosure provides a method of treating cancer in a subject in need thereof, comprising administering to the subject an effective amount of a compound described herein, such as a compound of Formula I, or a pharmaceutically acceptable salt thereof.

In another aspect, the present invention provides a compound, such as a compound of Formula I, or a pharmaceutically acceptable salt thereof, for use as a medicament for the treatment of cancer.

### DETAILED DESCRIPTION OF THE INVENTION

The present invention includes compounds of formula I

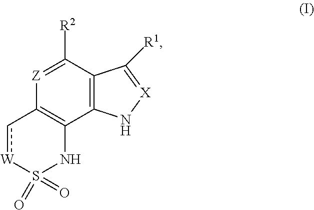

or a pharmaceutically acceptable salt thereof, wherein:
W is NR^{a} or CR^{a}R^{b};
R^{b} is hydrogen, or (C₁-C₄)alkyl;
R^{a} is selected from H, (C₁-C₆)alkyl, —(C₁-C₆)alkylOH, (C₁-C₆)alkoxy(C₁-C₆)alkyl), (C₁-C₆)haloalkyl, (C₂-C₈)alkenyl, cycloalkyl((C₀-C₆)alkyl), aryl((C₀-C₆)alkyl), heterocycloalkyl((C₀-C₆)alkyl), heteroaryl((C₀-C₆)alkyl), ((C₀-C₆)alkyl)amino((C₁-C₆)alkyl), and ((C₁-C₆)alkyl)carbonylamino((C₁-C₆)alkyl), wherein R^{a} is substituted with 0, 1, 2, or 2 R⁴ substituents;
each R⁴ independently is selected from (C₁-C₆)alkylcarbonyl, cyano, (C₁-C₆)haloalkyl, halogen, oxo, (C₁-C₆)alkyl, (C₁-C₆)alkoxy((C₀-C₆)alkyl), —SO₂(C₁-C₆)alkyl, amino, hydroxy, amino(C₁-C₆)akylcarbonyl, (C₁-C₆)alkylcarboxy, and —(C₁-C₆)alkylOH;
each n independently is 1, 2, or 3;
Z is CH, or N;
X is CH or N;
R¹ is H, (C₁-C₆)alkyl, cyano, —(C₁-C₆)OH, halogen, or (C₁-C₆)haloalkyl; and
R² is H, (C₁-C₆)alkyl, cyano, —(C₁-C₆)OH, halogen, or (C₁-C₆)haloalkyl.

In a first embodiment of the invention, R¹ is hydrogen, (C₁-C₆)alkyl, cyano, or halogen, and the other groups are as provided in the general formula above.

In a second embodiment of the invention, R¹ is methyl, ethyl, propyl, isopropyl, cyano, hydrogen, chloro, or fluoro, and the other groups are as provided in the general formula above.

In a third embodiment of the invention R¹ is hydrogen, cyano, or chloro, and the other groups are as provided in the general formula above.

In a fourth embodiment of the invention, R² is methyl, ethyl, propyl, isopropyl, cyano, hydrogen, chloro, or fluoro, and the other groups are as provided in the general formula above, or as in the first through third embodiments.

In a fifth embodiment of the invention, R² is methyl, hydrogen, chloro, or fluoro and the other groups are as provided in the general formula above, or as in the first through third embodiments.

In a sixth embodiment of the invention X is CH and the other groups are as provided in the general formula above, or as in the first through fifth embodiments.

In a seventh embodiment, X is N and the other groups are as provided in the general formula above, or as in the first through fifth embodiments.

In an eighth embodiment, W is NR^{a} and the other groups are as provided in the general formula above, or as in the first through seventh embodiments.

In a ninth embodiment of the invention, W is CR^{a}R^{b} and the other groups are as provided in the general formula above, or as in the first through seventh embodiments.

In a tenth embodiment of the invention, R^{b} is hydrogen, methyl, ethyl, propyl, tert-butyl or isopropyl and the other groups are as provided in the general formula above, or as in the first through ninth embodiments.

In a eleventh embodiment of the invention, R^{b} is hydrogen, or methyl and the other groups are as provided in the general formula above, or as in the first through ninth embodiments.

In a twelfth embodiment of the invention, R^{a} is selected from methylcarbonyl, ethylcarbonyl, hydrogen, cyano, difluoromethyl, 2,2,2,-trifluoroethyl, fluoromethyl, fluoro, chloro, oxo, methyl, ethyl, propyl, isopropyl, butyl, methoxy, ethoxy, methylsulfonyl, ethylsulfonyl, hydroxymethyl, hydroxyethyl, hydroxy, amino, and trifluoromethyl and the other groups are as provided in the general formula above, or as in the first through eleventh embodiments.

In a thirteenth embodiment of the invention, R^{a} is selected from methylcarbonyl, hydrogen, cyano, difluoromethyl, fluoro, chloro, oxo, methyl, methoxy, methylsulfonyl, hydroxy, amino, and trifluoromethyl and the other groups are as provided in the general formula above, or as in the first through eleventh embodiments.

Non-limiting examples of the Compounds of Formula I include compounds 1-100 or a pharmaceutically acceptable salt thereof, as set forth in the Examples:
6,7-dichloro-3-(2-methoxyethyl)-4,9-dihydro-1H-pyrrolo[3,2-h][2,1,3]benzothiadiazine 2,2-dioxide;
6-chloro-3-(2-methoxyethyl)-4,9-dihydro-1H-pyrrolo[3,2-h][2,1,3]benzothiadiazine 2,2-dioxide;
6,7-dichloro-1,3,4,9-tetrahydropyrrolo[3,2-h][2,1,3]benzothiadiazine 2,2-dioxide,
6,7-dichloro-3-cyclohexyl-4,9-dihydro-1H-pyrrolo[3,2-h][2,1,3]benzothiadiazine 2,2-dioxide;
6,7-dichloro-1,3,4,9-tetrahydropyrrolo[3,2-h][2,1]benzothiazine 2,2-dioxide;
3-benzyl-6,7-dichloro-4,9-dihydro-1H-pyrrolo[3,2-h][2,1,3]benzothiadiazine 2,2-dioxide;
6,7-dichloro-3-phenyl-4,9-dihydro-1H-pyrrolo[3,2-h][2,1,3]benzothiadiazine 2,2-dioxide;
6-chloro-3-(2-methoxyethyl)-2,2-dioxo-4,9-dihydro-1H-pyrrolo[3,2-h][2,1,3]benzothiadiazine-7-carbonitrile;
7-chloro-3-propyl-1,9-dihydropyrrolo[3,2-h][2,1]benzothiazine 2,2-dioxide;
7-chloro-3-propyl-1,3,4,9-tetrahydropyrrolo[3,2-h][2,1]benzothiazine 2,2-dioxide;
N-[(7-amino-3,4-dichloro-1H-indol-6-yl)methyl]-N-[(3-chlorophenyl)methyl]methanesulfonamide;
6,7-dichloro-3-[(6-chloro-3-pyridyl)methyl]-4,9-dihydro-1H-pyrrolo[3,2-h][2,1,3]benzothiadiazine 2,2-dioxide;
6,7-dichloro-3-[(4-chlorophenyl)methyl]-4,9-dihydro-1H-pyrrolo[3,2-h][2,1,3]benzothiadiazine 2,2-dioxide;
6,7-dichloro-3-[(2-chlorophenyl)methyl]-4,9-dihydro-1H-pyrrolo[3,2-h][2,1,3]benzothiadiazine 2,2-dioxide;
6,7-dichloro-3-methyl-4,9-dihydro-1H-pyrrolo[3,2-h][2,1,3]benzothiadiazine 2,2-dioxide;
3-benzyl-6,7-dichloro-1,3,4,9-tetrahydropyrrolo[3,2-h][2,1]benzothiazine 2,2-dioxide;
5-[(6,7-dichloro-2,2-dioxo-4,9-dihydro-1H-pyrrolo[3,2-h][2,1,3]benzothiadiazin-3-yl)methyl]-1H-pyridin-2-one;
6,7-dichloro-3-(4-fluorobutyl)-4,9-dihydro-1H-pyrrolo[3,2-h][2,1,3]benzothiadiazine 2,2-dioxide;
6,7-dichloro-3-(2-pyridylmethyl)-4,9-dihydro-1H-pyrrolo[3,2-h][2,1,3]benzothiadiazine 2,2-dioxide;
6,7-dichloro-3-(4-pyridylmethyl)-4,9-dihydro-1H-pyrrolo[3,2-h][2,1,3]benzothiadiazine 2,2-dioxide;
6,7-dichloro-3-[(6-methoxy-3-pyridyl)methyl]-4,9-dihydro-1H-pyrrolo[3,2-h][2,1,3]benzothiadiazine 2,2-dioxide;
6,7-dichloro-3-(2-methoxyethyl)-1,3,4,9-tetrahydropyrrolo[3,2-h][2,1]benzothiazine 2,2-dioxide;
6,7-dichloro-3-(1-phenylethyl)-4,9-dihydro-1H-pyrrolo[3,2-h][2,1,3]benzothiadiazine 2,2-dioxide;
2-(6,7-dichloro-2,2-dioxo-4,9-dihydro-1H-pyrrolo[3,2-h][2,1,3]benzothiadiazin-3-yl)ethanol;
6,7-dichloro-3-(4,4,5,5,5-pentafluoropentyl)-4,9-dihydro-1H-pyrrolo[3,2-h][2,1,3]benzothiadiazine 2,2-dioxide;
6,7-dichloro-3-(3-pyridylmethyl)-4,9-dihydro-1H-pyrrolo[3,2-h][2,1,3]benzothiadiazine 2,2-dioxide;
6,7-dichloro-3-[(2-chloro-3-pyridyl)methyl]-4,9-dihydro-1H-pyrrolo[3,2-h][2,1,3]benzothiadiazine 2,2-dioxide;
2-(6,7-dichloro-2,2-dioxo-4,9-dihydro-1H-pyrrolo[3,2-h][2,1,3]benzothiadiazin-3-yl)-N-methyl-ethanamine; 2,2,2-trifluoroacetic acid;
(3R)-6,7-dichloro-3-(2-methoxyethyl)-1,3,4,9-tetrahydropyrrolo[3,2-h][2,1]benzothiazine 2,2-dioxide;
(3S)-6,7-dichloro-3-(2-methoxyethyl)-1,3,4,9-tetrahydropyrrolo[3,2-h][2,1]benzothiazine 2,2-dioxide;
6,7-dichloro-1,3,4,9-tetrahydropyrrolo[1,2]pyrido[3,5-d][1,2,6]thiadiazine 2,2-dioxide;
6,7-dichloro-3-(4,4,4-trifluorobutyl)-4,9-dihydro-1H-pyrrolo[3,2-h][2,1,3]benzothiadiazine 2,2-dioxide;
6,7-dichloro-3-(pyrimidin-5-ylmethyl)-4,9-dihydro-1H-pyrrolo[3,2-h][2,1,3]benzothiadiazine 2,2-dioxide;
6,7-dichloro-3-(oxetan-3-ylmethyl)-4,9-dihydro-1H-pyrrolo[3,2-h][2,1,3]benzothiadiazine 2,2-dioxide;
6,7-dichloro-3-[(3-chloro-4-pyridyl)methyl]-4,9-dihydro-1H-pyrrolo[3,2-h][2,1,3]benzothiadiazine 2,2-dioxide;
6,7-dichloro-3-[(5-methyl-1,3,4-oxadiazol-2-yl)methyl]-4,9-dihydro-1H-pyrrolo[3,2-h][2,1,3]benzothiadiazine 2,2-dioxide;
6,7-dichloro-3-[(1-methyl-1,2,4-triazol-3-yl)methyl]-4,9-dihydro-1H-pyrrolo[3,2-h][2,1,3]benzothiadiazine 2,2-dioxide;
6,7-dichloro-3-[(2-methyl-1,2,4-triazol-3-yl)methyl]-4,9-dihydro-1H-pyrrolo[3,2-h][2,1,3]benzothiadiazine 2,2-dioxide;
6,7-dichloro-3-(4-piperidylmethyl)-4,9-dihydro-1H-pyrrolo[3,2-h][2,1,3]benzothiadiazine 2,2-dioxide;
N-[2-(6,7-dichloro-2,2-dioxo-4,9-dihydro-1H-pyrrolo[3,2-h][2,1,3]benzothiadiazin-3-yl)ethyl]acetamide;
2-(6,7-dichloro-2,2-dioxo-4,9-dihydro-1H-pyrrolo[3,2-h][2,1,3]benzothiadiazin-3-yl)-N,N-dimethyl-ethanamine;
6,7-dichloro-3-(pyridazin-3-ylmethyl)-4,9-dihydro-1H-pyrrolo[3,2-h][2,1,3]benzothiadiazine 2,2-dioxide;
6,7-dichloro-3-[(1-methylpyrazol-4-yl)methyl]-4,9-dihydro-1H-pyrrolo[3,2-h][2,1,3]benzothiadiazine 2,2-dioxide;
6,7-dichloro-3-(tetrahydropyran-4-ylmethyl)-4,9-dihydro-1H-pyrrolo[3,2-h][2,1,3]benzothiadiazine 2,2-dioxide;
1-[4-[(6,7-dichloro-2,2-dioxo-4,9-dihydro-1H-pyrrolo[3,2-h][2,1,3]benzothiadiazin-3-yl)methyl]-1-piperidyl]ethenone;
2-[(6,7-dichloro-2,2-dioxo-4,9-dihydro-1H-pyrrolo[3,2-h][2,1,3]benzothiadiazin-3-yl)methyl]benzonitrile;
1-[3-[(6,7-dichloro-2,2-dioxo-4,9-dihydro-1H-pyrrolo[3,2-h][2,1,3]benzothiadiazin-3-yl)methyl]azetidin-1-yl]ethenone;
6,7-dichloro-3-[(6-methyl-3-pyridyl)methyl]-4,9-dihydro-1H-pyrrolo[3,2-h][2,1,3]benzothiadiazine 2,2-dioxide;
6,7-dichloro-3-[[6-(trifluoromethyl)-3-pyridyl]methyl]-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]benzonitrile;
3-[(6,7-dichloro-2,2-dioxo-4,9-dihydro-1H-pyrrolo[3,2-h][2,1,3]benzothiadiazin-3-yl)methyl]benzonitrile;
6,7-dichloro-3-(pyrimidin-4-ylmethyl)-4,9-dihydro-1H-pyrrolo[3,2-h][2,1,3]benzothiadiazine 2,2-dioxide;
6,7-dichloro-3-[[6-(difluoromethyl)-3-pyridyl]methyl]-4,9-dihydro-1H-pyrrolo[3,2-h][2,1,3]benzothiadiazine 2,2-dioxide;
6,7-dichloro-3-[(4-chloro-3-pyridyl)methyl]-4,9-dihydro-1H-pyrrolo[3,2-h][2,1,3]benzothiadiazine 2,2-dioxide;
6,7-dichloro-3-[(4-methyl-3-pyridyl)methyl]-4,9-dihydro-1H-pyrrolo[3,2-h][2,1,3]benzothiadiazine 2,2-dioxide;
6,7-dichloro-3-((5-chloropyridin-3-yl)methyl)-1,3,4,9-tetrahydro-[1,2,6]thiadiazino[4,3-g]indole 2,2-dioxide;
6-[(6,7-dichloro-2,2-dioxo-4,9-dihydro-1H-pyrrolo[3,2-h][2,1,3]benzothiadiazin-3-yl)methyl]-1H-pyridin-2-one;
6-chloro-2,2-dioxo-1,3,4,9-tetrahydropyrrolo[3,2-h][2,1,3]benzothiadiazine-7-carbonitrile;
6,7-dichloro-3-(pyrazin-2-ylmethyl)-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]-1H-pyridin-2-one;
4-[(6,7-dichloro-2,2-dioxo-4,9-dihydro-1H-pyrrolo[3,2-h][2,1,3]benzothiadiazin-3-yl)methyl]-1-methyl-pyridin-2-one;
6-[(6,7-dichloro-2,2-dioxo-4,9-dihydro-1H-pyrrolo[3,2-h][2,1,3]benzothiadiazin-3-yl)methyl]-1-methyl-pyridin-2-one;
6,7-dichloro-3-[(2-chloro-4-pyridyl)methyl]-4,9-dihydro-1H-pyrrolo[3,2-h][2,1,3]benzothiadiazine 2,2-dioxide;
6,7-dichloro-3-(2-methoxyethyl)-4,9-dihydro-1H-pyrazolo[4,3-h][2,1,3]benzothiadiazine 2,2-dioxide;
5-[(6,7-dichloro-2,2-dioxo-4,9-dihydro-1H-pyrrolo[3,2-h][2,1,3]benzothiadiazin-3-yl)methyl]-1-methyl-pyridin-2-one;
6,7-dichloro-3,3-dimethyl-4,9-dihydro-1H-pyrrolo[3,2-h][2,1]benzothiazine 2,2-dioxide;
6,7-dichloro-3-(pyridazin-4-ylmethyl)-4,9-dihydro-1H-pyrrolo[3,2-h][2,1,3]benzothiadiazine 2,2-dioxide;
6,7-dichloro-3-(3-methoxypropyl)-4,9-dihydro-1H-pyrrolo[3,2-h][2,1,3]benzothiadiazine 2,2-dioxide;
6,7-dichloro-3-[(4-fluorophenyl)methyl]-4,9-dihydro-1H-pyrrolo[3,2-h][2,1,3]benzothiadiazine 2,2-dioxide;
6,7-dichloro-3-[(2-fluorophenyl)methyl]-4,9-dihydro-1H-pyrrolo[3,2-h][2,1,3]benzothiadiazine 2,2-dioxide;
6,7-dichloro-3-(2-morpholinoethyl)-4,9-dihydro-1H-pyrrolo[3,2-h][2,1,3]benzothiadiazine 2,2-dioxide;
6,7-dichloro-3-[(1-methylsulfonyl-4-piperidyl)methyl]-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]phenol;
2-[(6,7-dichloro-2,2-dioxo-4,9-dihydro-1H-pyrrolo[3,2-h][2,1,3]benzothiadiazin-3-yl)methyl]aniline;
6,7-dichloro-3-[(3-fluorophenyl)methyl]-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]aniline;
6,7-dichloro-3-(3-methylsulfonylpropyl)-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]aniline;
7-chloro-6-fluoro-3-(2-methoxyethyl)-4,9-dihydro-1H-pyrrolo[3,2-h][2,1,3]benzothiadiazine 2,2-dioxide;
7-chloro-3-tetrahydropyran-3-yl-4,9-dihydro-1H-pyrrolo[3,2-h][2,1,3]benzothiadiazine 2,2-dioxide;
7-chloro-3-(2-methoxyethyl)-4,9-dihydro-1H-pyrrolo[3,2-h][2,1,3]benzothiadiazine 2,2-dioxide;
7-chloro-3-(3-pyridyl)-4,9-dihydro-1H-pyrrolo[3,2-h][2,1,3]benzothiadiazine 2,2-dioxide;
6,7-dichloro-3-[(1-methyl-4-piperidyl)methyl]-4,9-dihydro-1H-pyrrolo[3,2-h][2,1,3]benzothiadiazine 2,2-dioxide;
6,7-dichloro-3-(2-phenylethyl)-4,9-dihydro-1H-pyrrolo[3,2-h][2,1,3]benzothiadiazine 2,2-dioxide;
6,7-dichloro-3-[(3,3-difluorocyclobutyl)methyl]-4,9-dihydro-1H-pyrrolo[3,2-h][2,1,3]benzothiadiazine 2,2-dioxide;
6,7-dichloro-3-(tetrahydrofuran-3-ylmethyl)-4,9-dihydro-1H-pyrrolo[3,2-h][2,1,3]benzothiadiazine 2,2-dioxide;
7-chloro-3-(2-methoxyethyl)-6-methyl-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]cyclobutanol;
6,7-dichloro-3-(tetrahydrofuran-2-ylmethyl)-4,9-dihydro-1H-pyrrolo[3,2-h][2,1,3]benzothiadiazine 2,2-dioxide;
6,7-dichloro-3-(3-piperidylmethyl)-4,9-dihydro-1H-pyrrolo[3,2-h][2,1,3]benzothiadiazine 2,2-dioxide;
1-[3-[(6,7-dichloro-2,2-dioxo-4,9-dihydro-1H-pyrrolo[3,2-h][2,1,3]benzothiadiazin-3-yl)methyl]-1-piperidyl]ethenone;
2-(6,7-dichloro-2,2-dioxo-4,9-dihydro-1H-pyrrolo[3,2-h][2,1,3]benzothiadiazin-3-yl)-1-morpholino-ethanone;
3-allyl-6,7-dichloro-4,9-dihydro-1H-pyrrolo[3,2-h][2,1,3]benzothiadiazine 2,2-dioxide;
6,7-dichloro-3-[2-(oxetan-3-yl)ethyl]-4,9-dihydro-1H-pyrrolo[3,2-h][2,1,3]benzothiadiazine 2,2-dioxide;
6,7-dichloro-3-(tetrahydropyran-3-ylmethyl)-4,9-dihydro-1H-pyrrolo[3,2-h][2,1,3]benzothiadiazine 2,2-dioxide;
6,7-dichloro-3-(tetrahydropyran-2-ylmethyl)-4,9-dihydro-1H-pyrrolo[3,2-h][2,1,3]benzothiadiazine 2,2-dioxide;
3-chloro-4-[(6,7-dichloro-2,2-dioxo-4,9-dihydro-1H-pyrrolo[3,2-h][2,1,3]benzothiadiazin-3-yl)methyl]-1H-pyridin-2-one;
5-chloro-4-[(6,7-dichloro-2,2-dioxo-4,9-dihydro-1H-pyrrolo[3,2-h][2,1,3]benzothiadiazin-3-yl)methyl]-1H-pyridin-2-one;
6-fluoro-3-(2-methoxyethyl)-2,2-dioxo-4,9-dihydro-1H-pyrrolo[3,2-h][2,1,3]benzothiadiazine-7-carbonitrile; or
3-(2-methoxyethyl)-2,2-dioxo-4,9-dihydro-1H-pyrrolo[3,2-h][2,1,3]benzothiadiazine-7-carbonitrile.

Unless defined otherwise, all technical and scientific terms used herein have the same meaning as is commonly understood by one of skill in the art to which this invention belongs.

As used in the specification and claims, the singular form “a”, “an” and “the” include plural references unless the context clearly dictates otherwise.

Unless defined otherwise, the term “Cx-Cy” when used in conjunction with a chemical moiety, such as alkyl, alkenyl, or alkynyl is meant to include groups that contain from x to y carbons in the chain. For example, the term “(Cx-Cy) alkyl” refers to substituted or unsubstituted saturated hydrocarbon groups, including straight-chain alkyl and branched-chain alkyl groups that contain from x to y carbons in the chain.

The term “C₀” or “C0” as employed in expressions such as “(C₀-C₆)alkyl” and C0-6alkyl” means a direct covalent bond; or when the term appears at the terminus of a substituent, C₀₋₆ alkyl means hydrogen. Similarly, when an integer defining the presence of a certain number of atoms in a group is equal to zero, it means that the atoms adjacent thereto are connected directly by a bond. For example, in the structure

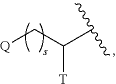

wherein s is an integer equal to zero, 1 or 2, the structure is

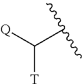

when s is zero.

The term “alkyl” as used herein refers to saturated linear or branched-chain monovalent hydrocarbon radicals. In one embodiment, an alkyl group contains from about 1 to about 10 carbon atoms. The term “(C₁-C₁₀)alkyl” as used herein refers to saturated linear or branched-chain monovalent hydrocarbon radicals of one to 10 carbon atoms, respectively. The term “(C₁-C₄)alkyl” as used herein refers to saturated linear or branched-chain monovalent hydrocarbon radicals of one to four carbon atoms, respectively. Illustrative examples of (C₁-C₄)alkyl include methyl, ethyl, 1-propyl, 2-propyl, I-butyl, 2-methyl-1-propyl, 2-butyl, and 2-methyl-2-propyl. The term (C₁-C₆)alkyl as used herein refers to saturated linear or branched-chain monovalent hydrocarbon radicals of one to six carbon atoms, respectively. Illustrative examples of (C₁-C₆)alkyl include, but are not limited to, methyl, ethyl, 1-propyl, 2-propyl, I-butyl, 2-methyl-1-propyl, 2-butyl, 2-methyl-2-propyl, 1-hexyl, 2-hexyl, 3-hexyl, 3,3-dimethylbutabyl, 2,2-dimethylbutanyl, 2,3-dimethylbutanyl, 2-methylpentanyl, 3-methylpentanyl, and 4-methylpentanyl.

The term “alkoxy” refers to an alkyl (carbon and hydrogen chain) group singularly bonded to oxygen (R—O). Non-limiting examples of alkoxy are methoxy (CH₃ O—)·, ethoxy (CH₃ CH₂ O—) and butoxy (CH₃ CH₂ CH₂ O—).

The term “carbonyl” means a functional group composed of a carbon atom double-bonded to an oxygen atom, C═O.

The term “carboxy” or “carboxyl” means a carbon atom double bonded to an oxygen atom and single bonded to a hydroxyl group (—COOH).

“Cycloalkyl” or “C₃₋₁₂ cycloalkyl” means any univalent non-aromatic radicals derived from a monocyclic or bicyclic ring system having 3 to 12 ring carbons atoms and may be fully saturated, or partially unsaturated; said ring system may be (a) a C₃ to a C₈ monocyclic, fully saturated or partially unsaturated ring, or (b) a bicyclic ring. Here, the point of attachment for a “cycloalkyl” to the rest of the molecule is on the saturated ring. Bicyclic cycloalkyl ring systems include fused ring systems, where two rings share two atoms (e.g. decalin), spiro ring systems where two rings share one atom (e.g. spiro[4.5]decanyl) and bridge groups (e.g., norbornane).

Additional examples within the above meaning include, but are not limited to univalent radicals of cyclopropyl, cyclobutyl, cyclopentyl, cyclohexyl, cycloheptyl, bicyclo[2.2.2]octanyl, bicyclo[1.1.1]pentanyl, bicyclo [2.2.1]heptanyl, [1.1.1]-bicyclo pentane, 1-decalinyl, spiro[2.4]heptyl, spiro[2.2]pentyl, 2,3-dihydro-1H-indenyl, and norbornyl.

The term “C₃₋₈ cycloalkyl” (or “C₃-C₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The terms “C₃₋₇ cycloalkyl”, “C₃₋₆ cycloalkyl”, “C₅₋₇ cycloalkyl” and the like have analogous meanings.

The term “(C₁-C₆)fluoroalkyl” as used herein refers to saturated linear or branched-chain monovalent hydrocarbon radicals of one to six carbon atoms substituted with one or more fluorine atoms. Illustrative examples include, but are not limited to, CHF₂, CH₂F, CF₃, CH₂CF₃, CF₂CH₃, CHFCH₃, CF(CH₃)₂, CH(CF₃)₂, CHFCF₃ and CF₂CF₃.

The term “(C₂-C₈)alkenyl” as used herein refers to straight or branched hydrocarbon chain radicals containing at least one double bond and having from two to five carbon atoms. A (C₂-C₈) alkenyl is attached to the rest of a molecule by a single bond through an sp² hybridized carbon. Illustrative examples of (C₂-C₈)alkenyl include, but are not limited to, ethenyl (i.e., vinyl), prop-1-enyl, but-1-enyl, pent-1-enyl, penta-1,4-dienyl.

The term “heteroaryl”, as used herein, represents a stable monocyclic, bicyclic or tricyclic ring system containing 5-14 carbon atoms and containing at least one ring heteroatom selected from N, S (including SO and SO₂) and O, wherein at least one of the heteroatom containing rings is aromatic. In the case of a heteroaryl ring system where one or more of the rings are saturated and contain one or more N atoms, the N can be in the form of quarternary amine. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzothiazolyl, benzo[d]isothiazole, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyrrolyl, pyrazolopyrimidinyl, pyridazinyl, pyridyl, pyrimidyl, pyrimidinyl, pyrrolyl, quinazolinyl, quinolyl, quinoxalinyl, tetrazolyl, tetrazolopyridyl, thiadiazolyl, 5H-pyrrolo[3,4-b]pyridine, thiazolyl, thienyl, triazolyl, triazinyl, benzothiazolyl, benzothienyl, quinolinyl, quinazolinyl, and isoquinolinyl, and oxazolyl. If the heteroaryl contains nitrogen atoms, it is understood that the corresponding N-oxides thereof are also encompassed by this definition.

Examples of 5-6 member heteraryls containing at least one ring heteroatom selected from N, S (including SO and SO₂) and O, include: furanyl, imidazolyl, isothiazolyl, isoxazolyl, oxadiazolyl, oxazolyl, oxazolinyl, isoxazolinyl, pyranyl, pyrazinyl, pyrazolyl, pyrrolyl, pyridazinyl, pyridyl, pyrimidyl, pyrimidinyl, pyrrolyl, tetrazolyl, thiadiazolyl, thiazolyl, thienyl, triazolyl, triazinyl, and oxazolyl.

The term “heterocycloalkyl,” as used herein, refers to a non-aromatic saturated monocyclic or multicyclic ring system comprising 3 to about 14 ring atoms, wherein from 1 to 4 of the ring atoms are independently O, S, or N, and the remainder of the ring atoms are carbon atoms. There are no adjacent oxygen and/or sulfur atoms present in the ring system. A heterocycloalkyl group can be joined via a ring carbon or ring nitrogen atom. Said ring system may be (a) a saturated monocyclic ring or a partially unsaturated ring, or (b) a bicyclic ring system having at one saturated ring with at least one ring atom that is independently O, S, or N. The other ring of the bicyclic system (b) may be saturated or partially unsaturated. For a bicyclic system, the rings are fused across two adjacent ring carbon atoms (e.g., decahydroisoquinoline, 2,3-dihydro-1H-benzo[d]imidazolyl, isoindolinyl), at one ring carbon atom (e.g., 1,4-dioxaspiro[4.5]decane), or are bridged groups (e.g., 2,5-diazabicyclo[2.2.1]heptyl, quinuclidinyl).

In one embodiment, a heterocycloalkyl group is monocyclic and has from about 3 to about 7 ring atoms. In another embodiment, a heterocycloalkyl group is monocyclic has from about 5 to about 8 ring atoms. In another embodiment, a heterocycloalkyl group is bicyclic and has from about 8 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, 1,2-dihydropyridyl (1,2-dihydropyridinyl), beta lactam, gamma lactam, delta lactam, beta lactone, gamma lactone, delta lactone, and pyrrolidinone, and oxides thereof and all isomers thereof.

The term “phenyl” as used herein refers to a radical with the formula C₆H.

The term “halogen” includes fluoro, chloro, bromo and iodo.

The term “oxy” means an oxygen (O) atom. The term “thio” means a sulfur (S) atom.

The term “oxo” means “═O”. The term “carbonyl” means “C═O.”

The term “substituted” as used herein refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

The term “optional” or “optionally” as used herein means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

By “pharmaceutically acceptable” is meant that the ingredients of the pharmaceutical composition must be compatible with each other and not deleterious to the recipient thereof.

Where any amine is present in the compound, the N atom may be optionally in the form of a quaternary amine having one or more appropriate additional substitutions, as further described herein.

When any variable (e.g., n, R^{a}, R^{b}, etc.) occurs more than one time in any constituent or in Formula (I), its definition on each occurrence is independent of its definition at every other occurrence. Also, combinations of substituents and/or variables are permissible only if such combinations result in stable compounds.

When any ring atom is specified as being optionally substituted with, or in a specified form, for example, S substituted with oxo groups, or N in the form of a N-oxide, this does not preclude the substitution of any ring atom with the other listed optional substituents when not substituted with oxo groups or in the form of a N-oxide.

“Celite®” (Fluka) diatomite is diatomaceous earth, and can be referred to as “celite”.

By “stable compound” or “stable structure” is meant a compound that is sufficiently robust to survive isolation to a useful degree of purity from a reaction mixture, and formulation into an efficacious therapeutic agent. The compounds of the present invention are limited to stable compounds embraced by Formula (I).

The term “compound” refers to the compound and, in certain embodiments, to the extent they are stable, any hydrate or solvate thereof. A hydrate is the compound complexed with water, and a solvate is the compound complexed with an organic solvent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

Under standard nomenclature used throughout this disclosure, the terminal portion of the designated side chain is preceded by the adjacent functionality toward the point of attachment. For example, a C1-5 alkylcarbonylamino C1-6 alkyl substituent is equivalent to

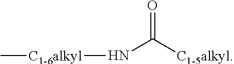

Structural representations of compounds having substituents terminating with a methyl group may display the terminal methyl group either using the characters “CH₃”, e.g. “—CH₃” or using a straight line representing the presence of the methyl group, e.g., “-”, i.e.,

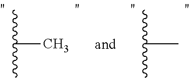

have equivalent meanings.

For variable definitions containing terms having repeated terms, e.g., (CRᵢRⱼ)ᵣ, where r is the integer 2, Rᵢ is a defined variable, and Rⱼ is a defined variable, the value of Rᵢ may differ in each instance in which it occurs, and the value of Rⱼ may differ in each instance in which it occurs. For example, if Rᵢ and Rⱼ are independently selected from the group consisting of methyl, ethyl, propyl and butyl, then (CRᵢRⱼ)² can be

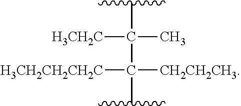

Unless expressly stated to the contrary, all ranges cited herein are inclusive. For example, a heteroaromatic ring described as containing from “1 to 4 heteroatoms” means the ring can contain, 1, 2, 3 or r heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁-C₆ when used with a chain, for example an alkyl chains means that the chain can contain 1, 2, 3, 4, 5, or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

In choosing compounds of the present invention, one of ordinary skill in the art will recognize that the various substituents, i.e. R¹, R^{A}, etc., are to be chosen in conformity with well-known principles of chemical structure connectivity and stability.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a compound of Formula (I) or a pharmaceutically acceptable salt of the compound. The transformation may occur by various mechanisms (e.g., by metabolic or chemical processes), such as, for example, through hydrolysis in blood. For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If a compound of Formula (I) incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters, including those corresponding to both natural and non-natural amino acids (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvates, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The compound of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), arylalkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the compound of Formula (I) may be atropisomers (e.g., substituted biaryls) and are considered as part of this invention. Enantiomers can also be directly separated using chiral chromatographic techniques.

It is also possible that the compound of Formula (I) may exist in different tautomeric forms, and all such forms are embraced within the scope of the invention. For example, all keto-enol and imine-enamine forms of the compounds are included in the invention.

Unless otherwise indicated, all stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a compound of Formula (I) incorporates a double bond or a fused ring, both the cis- and trans-forms, as well as mixtures, are embraced within the scope of the invention.

When a substituent on a chiral carbon atom is depicted without specific stereochemistry (by using a straight line bond to a chiral center), it is to be understood that both the alpha and beta configurations of said substituent group are to be considered part of the present invention. For example, the compound of the present invention, which is drawn as follows:

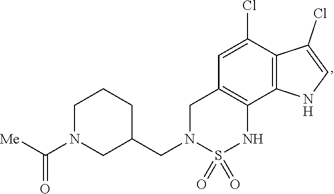 [SMILES: CC(=O)N1CCCC(CN2Cc3cc(Cl)c4c(Cl)c[nH]c4c3NS2(=O)=O)C1]

is understood to encompass both stereoisomers at the indicated chiral center located at the carbon atom attached to the carboxamide portion of the compound, the structures of which are as follows:

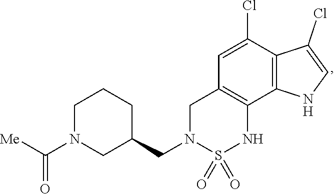 [SMILES: CC(=O)N1CCC[C@@H](CN2Cc3cc(Cl)c4c(Cl)c[nH]c4c3NS2(=O)=O)C1]

(R)-1-(3-((6,7-dichloro-2,2-dioxido-4,9-dihydro-[1,2,6]thiadiazino[4,3-g]indol-3(1H)-yl)methyl)piperidin-1-yl)ethan-1-one, and

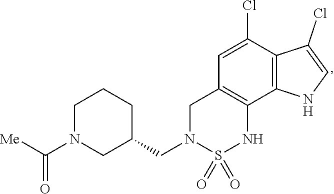 [SMILES: CC(=O)N1CCC[C@H](CN2Cc3cc(Cl)c4c(Cl)c[nH]c4c3NS2(=O)=O)C1]

(S)-1-(3-((6,7-dichloro-2,2-dioxido-4,9-dihydro-[1,2,6]thiadiazino[4,3-g]indol-3(1H)-yl)methyl)piperidin-1-yl)ethan-1-one.

In the Examples section below, compounds of the present invention that have been purified as individual stereoisomers are sometimes depicted in non-stereospecific form but identified using one or more of the terms: “diastereomer 1,” “diastereomer 2,” “isomer 1,” “isomer 2,” “first eluding enantiomer”, “enantiomer A” and “enantiomer B.” In this instance, the absolute stereochemistry of each isolated diastereomer and enantiomeric center has not been determined and the terms used above are used to represent each individual purified stereochemically pure compound.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

In another embodiment, the Compounds of Formula (I) are in substantially purified form.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. It is recognized that one skilled in the art may affect the cancerous disorders by treating a patient presently afflicted with the disorders or by prophylactically treating a patient afflicted with such disorders with an effective amount of the compound of the present invention.

As used herein, “treatment”, “treatment of” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “degrader” as used herein refers to a compound having the ability to induce the degradation of a target protein. For example, a degrader may induce the ubiquitination and subsequent proteasomal degradation of a target protein. Targeted protein degradation can be undertaken for the purposes of inhibiting the biological function of the target protein, meaning that a degrader can be a member of a specific sub-class of antagonist. Compounds that potentiate the formation of a complex between a target protein and any portion of an E3 ubiquitin ligase complex are specifically included within this definition. Additionally included in “degrader” are heterobifunctional degraders and included any degraders that could be derived from this chemotype for the purpose of degrading any proteins other than RBM39.

An example of a heterobifunctional degrader is a PROTAC degrader using indisulam for degradation of BRD4 (See Li, Liang et al., “In vivo target protein degradation induced by PROTACs based on E3 ligase DCAF15”, Sig. Transduct Target Ther 5, 129 (2020).

The term “RBM39 degrader” as used herein refers to a compound, such as a compound of Formula I or II, having the ability to induce the degradation of RBM39 protein. For example, an RBM39 degrader may induce the ubiquitination and subsequent proteasomal degradation of RBM39 protein. Targeted protein degradation can be undertaken for the purposes of inhibiting the biological function of the target protein. Compounds that potentiate the formation of a complex between RBM39 protein and any portion of an E3 ubiquitin ligase complex are included within this definition.

The term “cell proliferation” refers to a phenomenon by which the cell number has changed as a result of division. This term also encompasses cell growth by which the cell morphology has changed (e.g., increased in size) consistent with a proliferative signal.

The term “subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

The term “substantially pure” means that the isolated material is at least 90% pure, and preferably 95% pure, and even more preferably 99% pure as assayed by analytical techniques known in the art.

For purposes of this specification, the following abbreviations have the indicated meanings:
Ac acetyl
ACN acetonitrile
AcO acetate
BOC t-butyloxycarbonyl
CBZ carbobenzoxy
CDI carbonyldiimidazole
DBU 1,8-Diazabicycloundec-7-ene
DCC 1,3-dicyclohexylcarbodiimide
DCE 1,2-dichloroethane
(dF(CF3)ppy) 2-(2,4-difluorophenyl)-5-trifluoromethylpyridine
DI de-ionized
DIAD Diisopropyl azodicarboxylate
DIBAL diisobutyl aluminum hydride
DIPEA or DIEA N,N-diisoproylethylamine, also known as Hunig's base
DMA dimethylacetamide
DMAP 4-(dimethylamino)pyridine
DMF dimethylformamide
DMF·DMA N,N-dimethylformamide dimethyl acetal
DMP Dess-Martin periodinane
DPPA Diphenylphosphoryl azide
DPPP 1,3-bis(diphenylphosphino)propane
Dtbbpy 4,4′-di-tert-butyl-2,2′-dipyridyl
EDC or EDCI 1-(3-dimethylaminopropyl)-3-ethylcarbodiimide hydrochloride
EDTA ethylenediaminetetraacetic acid, tetrasodium salt
EtOAc ethyl acetate
FAB fast atom bombardment
FMOC 9-fluorenylmethoxycarbonyl
HMPA hexamethylphosphoramide
HATU O-(7-Azabenzotriazol-1-yl)-N,N,N′,N′-tetramethyluronium hexafluorophosphate
HOAt 1-Hydroxy-7-azabenzotriazole or 3H-[1,2,3]triazolo[4,5-b]pyridin-3-ol
HOBt 1-hydroxybenzotriazole
HRMS high resolution mass spectrometry
IBCF isobutyl chloroformate
KHMDS potassium hexamethyldisilazane
LC-MS Liquid chromatography-mass spectrometry
LDA lithium diisopropylamide
LiHMDS lithium hexamethyldisilazane
MCPBA metachloroperbenzoic acid
MMPP magnesium monoperoxyphthlate hexahydrate
Ms methanesulfonyl=mesyl
MsO methanefulfonate=mesylate
MTBE Methyl t-butyl ether
NBS N-bromosuccinimide
NCS N-chlorosuccinimide
NMM 4-methylmorpholine
NMP N-methylpyrrolidinone
NMR Nuclear magnetic resonance
PCC pyridinium chlorochromate
PDC pyridinium dichromate
Ph phenyl
PPTS pyridinium p-toluene sulfonate
pTSA p-toluene sulfonic acid
PyH·Br3 pyridine hydrobromide perbromide
r.t./RT room temperature
rac. racemic
T3P 2,4,6-Tripropyl-1,3,5,2,4,6-trioxatriphosphinane 2,4,6-trioxide
TEA triethylamine
TFA trifluoroacetic acid
TFAA trifluoroacetic acid anhydride
TfO trifluoromethanesulfonate=triflate
THF tetrahydrofuran
TLC thin layer chromatography
TMSCl trimethylsilyl chloride

The present invention is directed to a compound of the present invention or a pharmaceutically acceptable salt thereof for use in medicine. The present invention is further directed to a use of a compound of the present invention or a pharmaceutically acceptable salt thereof for the manufacture of a medicament for treating a disorder associated with RBM39 degradation function in a mammalian patient in need thereof. The present invention is further directed to a use of a compound of the present invention or a pharmaceutically acceptable salt thereof for the manufacture of a medicament for treating a disorder associated with cancers, such as, for example, acute myeloid leukemia (AML), colon, EZH2 mutant limphomas, and melanomia in a mammalian patient in need thereof.

The present invention is further directed to a use of the compound of formula 1 or a pharmaceutically acceptable salt thereof, in the preparation of a medicament for modulating at least one activity selected from RBM39 activity and DCAF 15 activity, in a patient in need thereof. In one embodiment, the activity is RBM39 activity. In another embodiment of the invention, the activity is DCAF 15 activity. In yet another embodiment of the invention, the modulation included both RBM39 activity and DCAF 15 activity where RBM39 protein degradation is the result of compound modulating (binding to) DCAF15 and recruiting RBM39.

The subject compounds may be further useful in a method for the prevention, treatment, control, amelioration, or reduction of risk of the diseases, disorders and conditions noted herein. The subject compounds are further useful in a method for the prevention, treatment, control, amelioration, or reduction of risk of the aforementioned diseases, disorders and conditions in combination with other agents.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefore, contemporaneously or sequentially with a compound of the present invention. When a compound of the present invention is used contemporaneously with one or more other drugs, a pharmaceutical composition in unit dosage form containing such other drugs and the compound of the present invention may be desirable. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly.

Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, AChEis (Aricept (donepezil)) and NMDA blocker Namenda (memantine), beta-secretase inhibitors, gamma-secretase inhibitors, HMG-CoA reductase inhibitors, NSAID's including ibuprofen, vitamin E, and anti-amyloid antibodies.

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compounds of Formula (I). The additional active agents also include free-acid, free-base and pharmaceutically acceptable salts of said additional active agents. Generally, any suitable additional active agent or agents, including chemotherapeutic agents or therapeutic antibodies may be used in any combination with a compound of Formula (I) in a single dosage formulation (a fixed dose drug combination), or in one or more separate dosage formulations which allows for concurrent or sequential administration of the active agents (co-administration of the separate active agents) to subjects. In addition, the compounds of Formulae (I)(or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes but is not limited to the combination of one or more compounds of Formula (I) with chemotherapeutic agents, therapeutic antibodies, and radiation treatment, to provide a synergistic or additive therapeutic effect.

In one embodiment, the combination therapies comprise chemotherapeutic agents. Many such agents are presently known in the art and can be used in combination with the compounds of Formula (I). In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), and Adriamycin as well. Non-limiting examples of chemotherapeutic agents include alkylating agents such as thiotepa and cyclosphosphamide (CYTOXANTM™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™ chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifiuridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxy tamoxifen, trioxifene, keoxifene, LY 117018, onapristone, and toremifene; and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); and topoisomerase inhibitor RFS 2000.

Where desired, the compounds of Formula (I) or pharmaceutical compositions containing such compounds can be used in combination with commonly prescribed anti-cancer drugs such as Herceptin®, Avastin®, Erbitux®, Rituxan®, Taxol®, Arimidex®, Taxotere®, ABVD, AVICINE, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, Antineoplastic, antitumorigenic herbs, apaziquone, atiprimod, azathioprine, belotecan, bendamustine, BIBW 2992, biricodar, brostallicin, bryostatin, buthionine sulfoximine, calyculin, cell-cycle nonspecific antineoplastic agents, dichloroacetic acid, discodermolide, elsamitrucin, enocitabine, epothilone, eribulin, everolimus, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pixantrone, proteasome inhibitor, rebeccamycin, resiquimod, rubitecan, SN-38, salinosporamide a, sapacitabine, swainsonine, talaporfin, tariquidar, tegafur-uracil, temozolimide, tesetaxel, triplatin tetranitrate, tris(2-chloroethyl)amine, troxacitabine, Vadimezan, Vinflunine, ZD6126 or Zosuquidar.

The present application further provides a method for using the compounds of Formula (I) or pharmaceutical compositions provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The compounds of Formula (I) or pharmaceutical compositions containing such compounds can be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors and MMP-9 (matrix-metalloproteinase 9) inhibitors, can be used in conjunction with a compound of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful matrix metalloproteinase inhibitors are described in WO 96/33172, WO 96/27583 European Patent Publication No. EP0818442, European Patent Publication No. EP1004578, WO 98/07697, WO 98/03516, WO 98/34918, WO 98/34915, WO 98/33768, WO 98/30566, European Patent Publication No. 606046, European Patent Publication No. 931788, WO 90/05719, WO 99/52910, WO 99/52889, WO 99/29667, WO 1999007675, European Patent Publication No. EP1786785, European Patent Publication No. EP1181017, U.S. Publication No. US20090012085, U.S. Pat. Nos. 5,863,949, 5,861,510, and European Patent Publication No. EP0780386. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in the combinations are AG-3340, RO 32-3555, and RS 13-0830.

The compounds of Formula (I) may also be used in co-therapies with other antineoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-la, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

The compounds of Formula (I) may further be used with VEGFR inhibitors.

In some embodiments, the combination comprises a composition of the present invention in combination with at least one anti-angiogenic agent. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-angiogenic agents include ERBITUX™, KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRESSA™ (gefitinib), TARCEVA™ (erlotinib), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”. Other anti-angiogenic agents include Campath, IL-8, B-FGF, Tek antagonists (Ceretti et al, U.S. Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., U.S. Publication No. 2002/0042368), specifically binding anti-eph receptor and/or anti-ephrin antibodies or antigen binding regions (U.S. Pat. Nos. 5,981,245; 5,728,813; 5,969,110; 6,596,852; 6,232,447; and 6,057,124), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide (Merck KGaA, Germany); pegaptanib octasodium, (Gilead Sciences, USA); alphastatin (BioActa, UK); M-PGA, ilomastat, (Arriva, USA); emaxanib, (Pfizer, USA); vatalanib (Novartis, Switzerland); 2-methoxyestradiol; TLC ELL-12 (Elan, Ireland); anecortave acetate (Alcon, USA); alpha-D148 Mab, (Amgen, USA); CEP-7055 (Cephalon, USA); anti-Vn Mab (Crucell, Netherlands) angiocidin (InKine Pharmaceutical, USA); KM-2550 (Kyowa Hakko, Japan); SU-0879 (Pfizer, USA); CGP-79787 (Novartis, Switzerland, EP 970070); fibrinogen-E fragment (BioActa, UK); TBC-1635 (Encysive Pharmaceuticals, USA); SC-236 (Pfizer, USA); metastatin (EntreMed, USA); maspin (Sosei, Japan); ER-68203-00 (IVAX, USA); benefin (Lane Labs, USA); Tz-93 (Tsumura, Japan); TAN-1120 (Takeda, Japan); FR-111142 (Fujisawa, Japan); platelet factor 4; vascular endothelial growth factor antagonist, (Borean, Denmark); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and Medlmmune, USA); enzastaurin hydrochloride (USAN), (Lilly, USA); CEP 7055, (Cephalon, USA and Sanofi-Synthelabo, France); BC 1, (Genoa Institute of Cancer Research, Italy); rBPI 21 and BPI-derived antiangiogenic (XOMA, USA); PI 88 (Progen, Australia); cetuximab, (Aventis, France); AVE 8062 (Ajinomoto, Japan); AS 1404, (Cancer Research Laboratory, New Zealand); SG 292, (Telios, USA); endostatin, (Boston Childrens Hospital, USA); ANGIOSTATIN (Boston Childrens Hospital, USA); AZD 6474, (AstraZeneca, UK); ZD 6126 (Angiogene Pharmaceuticals, UK); PPI 2458, (Praecis, USA); AZD 9935 (AstraZeneca, UK); AZD 2171 (AstraZeneca, UK); vatalanib (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); SDX 103, (University of California at San Diego, USA); PX 478, (ProlX, USA); METASTATIN (EntreMed, USA); troponin I, (Harvard University, USA); SU 6668, (SUGEN, USA); OXI 4503 (OXiGENE, USA); motuporamine C, (British Columbia University, Canada); CDP 791 (Celltech Group, UK); atiprimod (GlaxoSmithKline, UK); E 7820 (Eisai, Japan); CYC 381 (Harvard University, USA); AE 941 (Aeterna, Canada); urokinase plasminogen activator inhibitors; HIF-1 alfa inhibitors; angiocidin (InKine, USA); GW 2286 (GlaxoSmithKline, UK); EHT 0101 (ExonHit, France); CP 868596 (Pfizer, USA); CP 564959 (OSI, USA); CP 547632 (Pfizer, USA); 786034, (GlaxoSmithKline, UK); KRN 633 (Kirin Brewery, Japan); tumor necrosis factor-alpha inhibitors; KDR kinase inhibitors; combretastatin A4 prodrug (Arizona State University, USA); chondroitinase AC (IBEX, Canada); BAY RES 2690 (Bayer, Germany); tetrathiomolybdate (University of Michigan, USA); GCS 100 (Wayne State University, USA) CV 247 (Ivy Medical, UK); CKD 732, (Chong Kun Dang, South Korea); MAb, vascular endothelium growth factor, (Xenova, UK); irsogladine (Nippon Shinyaku, Japan); RG 13577 (Aventis, France); VE-cadherin-2 antagonists; vasostatin, (National Institutes of Health, USA); Flk-1, (ImClone Systems, USA); TZ 93 (Tsumura, Japan); TumStatin (Beth Israel Hospital, USA); forms of FLT 1 (vascular endothelial growth factor receptor 1); Tie-2 ligands (Regeneron, USA); and thrombospondin 1 inhibitor (Allegheny Health, USA).

Additional active compounds/agents that can be used in the treatment of cancers and that can be used in combination with one or more compounds of Formula (I) include: epoetin alfa; darbepoetin alfa; panitumumab; pegfilgrastim; palifermin; filgrastim; denosumab; ancestim or a pharmaceutically acceptable salt thereof.

The compounds of the present invention may also be used in combination with an additional pharmaceutically active compound that disrupts or inhibits RAS-RAF-ERK or PI3K-AKT-TOR signaling pathways. In other such combinations, the additional pharmaceutically active compound is a PD-1 and PD-L1 antagonist. The compounds or pharmaceutical compositions of the disclosure can also be used in combination with an amount of one or more substances selected from EGFR inhibitors, MEK inhibitors, ERK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAGl, and anti-OX40 agents, GITR agonists, CAR-T cells, and BiTEs.

EGFR inhibitors include, but are not limited to, small molecule antagonists, antibody inhibitors, or specific antisense nucleotide or siRNA. Useful antibody inhibitors of EGFR include cetuximab (Erbitux), panitumumab (Vectibix), zalutumumab, nimotuzumab, and matuzumab. Small molecule antagonists of EGFR include gefitinib, erlotinib, and lapatinib.

Antibody-based EGFR inhibitors include any anti-EGFR antibody or antibody fragment that can partially or completely block EGFR activation by its natural ligand. Non-limiting examples of antibody-based EGFR inhibitors include those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

MEK inhibitors include, but are not limited to, CI-1040, AZD6244, PD318088, PD98059, PD334581, RDEA119, ARRY-142886, ARRY-438162, and PD-325901.

PI3K inhibitors include, but are not limited to, wortmannin, 17-hydroxywortmannin analogs described in WO 06/044453, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), LY294002 (2-(4-Morpholinyl)-8-phenyl-4H-1-benzopyran-4-one available from Axon Medchem), PI 103 hydrochloride (3-[4-(4-morpholinylpyrido-[3′,2′:4,5]furo[3,2-d]pyrimidin-2-yl] phenol hydrochloride available from Axon Medchem), PIK 75 (N′-[(1E)-(6-bromoinddazo[1,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydro-imidazo[1,2-c]quinazolin-5-yl)-nicotinamide available from Axon Medchem), GDC-0941 bismesylate (2-(1H-Indazol-4-yl)-6-(4-methanesulfonyl-piperazin-1-ylmethyl)-4-morpholin-4-yl-thieno[3,2-d]pyrimidine bismesylate available from Axon Medchem), AS-252424 (5-[1-[5-(4-fluoro-2-hydroxy-phenyl)-furan-2-yl]-meth-(Z)-ylidene]-thiazolidine-2,4-dione available from Axon Medchem), and TGX-221 (7-Methyl-2-(4-morpholinyl)-9-[1-(phenylamino)ethyl]-4H-pyrido-[1,2-a]pyrindin-4-one available from Axon Medchem), XL-765, and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine; Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); and triciribine (TCN or API-2 or NCI identifier: NSC 154020; Yang et al. (2004) Cancer Res. 64, 4394-9).

TOR inhibitors include, but are not limited to, inhibitors include AP-23573, CCI-779, everolimus, RAD-001, rapamycin, temsirolimus, ATP-competitive TORC1/TORC2 inhibitors, including PI-103, PP242, PP30 and Torin 1. Other TOR inhibitors in FKBP12 enhancer; rapamycins and derivatives thereof, including: CCI-779 (temsirolimus), RAD001 (Everolimus; WO 9409010) and AP23573; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin, 40-epi-(tetrazolyt)-rapamycin (also called ABT578), 32-deoxorapamycin, 16-pentynyloxy-32(S)-dihydrorapanycin, and other derivatives disclosed in WO 05005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05016252).

MCl-1 inhibitors include, but are not limited to, AMG-176, MIK665, and S63845.

Proteasome inhibitors include, but are not limited to, Kyprolis® (carfilzomib), Velcade® (bortezomib), and oprozomib.

Immune therapies include, but are not limited to, anti-PD-1 agents, anti-PD-L1 agents, anti-CTLA-4 agents, anti-LAGl agents, and anti-OX40 agents.

Monoclonal antibodies include, but are not limited to, Darzalex® (daratumumab), Herceptin® (trastuzumab), Avastin® (bevacizumab), Rituxan® (rituximab), Lucentis® (ranibizumab), and Eylea® (aflibercept).

In some embodiments, the compounds of Formula (I) are used in combination with an anti-CTLA-4 antibody, e.g., ipilumumab.

The invention further relates to a method of treating cancer in a human patient comprising administration of a compound of the invention (i.e., a compound of Formula (I)) and a PD-1 antagonist to the patient. The compound of the invention and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-Li antibody, or antigen binding fragment thereof. In some embodiments, the PD-1 antagonist is pembrolizumab (KEYTRUDA™, Merck & Co., Inc., Kenilworth, NJ, USA), nivolumab (OPDIVO™, Bristol-Myers Squibb Company, Princeton, NJ, USA), cemiplimab (LIBTAYO™ Regeneron Pharmaceuticals, Inc., Tarrytown, NY, USA), atezolizumab (TECENTRIQ™ Genentech, San Francisco, CA, USA), durvalumab (IMFINZI™, AstraZeneca Pharmaceuticals LP, Wilmington, DE), or avelumab (BAVENCIO™, Merck KGaA, Darmstadt, Germany).

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks.

In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

The compounds of the invention can be used in combination with the agents disclosed herein or other suitable agents, depending on the condition being treated. Hence, in some embodiments the one or more compounds of the invention will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The terms “administration of” and or “administering a” compound should be understood to mean providing a compound of the invention or a prodrug of a compound of the invention to the individual in need of treatment.

The term “composition” as used herein is intended to encompass a product comprising specified ingredients in predetermined amounts or proportions, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by mixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art. By “pharmaceutically acceptable” it is meant the carrier, diluent or excipient must be compatible with the other ingredients of the formulation and not deleterious to the recipient thereof.

The subject compounds are further useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.001 to 10 mg/kg of body weight daily are administered to the patient, e.g., humans and elderly humans. The dosage range will generally be about 0.5 mg to 1.0 g per patient per day which may be administered in single or multiple doses. In one embodiment, the dosage range will be about 0.5 mg to 500 mg per patient per day; in another embodiment about 0.5 mg to 200 mg per patient per day; and in yet another embodiment about 5 mg to 50 mg per patient per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

While preferred embodiments of the present invention have been shown and described herein, it will be obvious to those skilled in the art that such embodiments are provided by way of example only. Numerous variations, changes and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing conditions as shown in the schemes and examples herein, as well as using other standard manipulations such as ester hydrolysis, cleavage of protecting groups, etc., as may be known in the literature or exemplified in the experimental procedures. Starting materials are made according to procedures known in the art or as illustrated herein.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood.

The representative examples of the compounds of the invention are illustrated in the following non-limiting schemes and Examples.

### GENERAL

Starting materials used were obtained from commercial sources or prepared in other examples, unless otherwisely noted. The progress of reactions was often monitored by TLC or LC-MS. The LC-MS was recorded using one of the following methods.

Preparative thin layer chromatography (PTLC) separations described herein were typically performed on 20×20 cm plates (500 micron thick silica gel).

Chromatographic purifications were typically performed using Biotage® Isolera One automated systems running Biotage® Isolera One 2.0.6 software (Biotage LLC, Charlotte, NC USA). Flow rates were the default values specified for the particular column in use. Reverse phase chromatography was performed using elution gradients of water and acetonitrile on Biotage® KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g. hexane, ethyl acetate, methylene chloride, methanol, acetone, chloroform, MTBE, etc.). The columns were Biotage® SNAP Cartridges containing KP-SIL or Biotage® SNAP Ultra (25 m spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹H NMR analyses of intermediates and exemplified compounds were typically performed on an Agilent Technologies 400/54 (Agilent Technologies, Santa Clara, CA, US) or Bruker® Ascend™ 400 spectrometer (Bruker BioSpin AG, Faellanden Switzerland) (operating at 400 MHz) at 298° K following standard operating procedure suggested by manufacturer. Reference frequency was set using TMS as an internal standard. Typical deuterated solvents were utilized as indicated in the individual examples.

LCMS analyses were typically performed using one of the two conditions listed below:
(1) LCMS spectra were taken on an Agilent Technologies 1260 Infinity coupled to 6120 Quadrupole spectrometer. The mobile phase for the LC was acetonitrile (A) and water (B) with 0.01% formic acid, and the eluent gradient was from 5-95% A in 6.0 min, 60-95% A in 5.0 min, 80-100% A in 5.0 min and 85-100% A in 10 min using a SBC18 50 mm×4.6 mm×2.7 m capillary column. Mass spectra (MS) were measured by electrospray ion-mass spectroscopy (ESI). All temperatures are in degrees Celsius unless otherwise noted; and
(2) LCMS analysis of intermediates and exemplified compounds was performed on an Agilent Technologies 1200 Series HPLC system coupled to an Agilent Technologies 6150 Quadrapole LC/MS detector. Analytes were detected by UV absorbance at 220 and 254 nm. Analyte ions were detected by mass spectrometry in both negative and positive modes (110-800 amu scan range, API-ES ionization). A long HPLC method was run on a Phenomenex® Kinetex 2.6μ C18 100 Å, 30×3.00 mm column (Phenomenex, Torrance, CA). The column temperature was set at 40° C. UV absorptions were detected at 220 and 254 nm. Samples were prepared as a solution in about 1:1 (v/v) acetonitrile:water mixture. Flow rate was about 0.80 mL/minute. Elution solvents were acetonitrile and water each containing 0.1% formic acid. In a typical run, a linear gradient starting with 5% acetonitrile and 95% water and ending with 95% acetonitrile and 5% water over 12 minutes was carried out. At the end of each run, the column was washed with 95% acetonitrile and 5% water for 2 minutes.

Typically, analytical HPLC mass spectrometry conditions were as follows:
1) LC1: Column: SB-C18 50 mm×4.6 mm×2.7 m; Temperature: 50° C.; Eluent: 5:95 v/v acetonitrile/water+0.01% formic acid in 6 min; Flow Rate: 1.5 mL/min; Injection 5 μL; Detection: PDA, 200-600 nm; MS: mass range 150-750 amu; positive ion electrospray ionization;
2) LC2: Column: SB-C18 50 mm×4.6 mm×2.7 m; Temperature: 50° C.; Eluent: 5:95 to 95:5 v/v acetonitrile/water+0.05% TFA over 3.00 min; Flow Rate: 1.5 mL/min; Injection 5 μL; Detection: PDA, 200-600 nm; MS: mass range 150-750 amu; positive ion electrospray ionization; and
3) LC3: Column: SB-C18 50 mm×4.6 mm×2.7 m; Temperature: 50° C.; Eluent: 10:90 to 98:2 v acetonitrile/water+0.05% TFA over 3.75 min; Flow Rate: 1.0 mL/min; Injection 10 μL; Detection: PDA, 200-600 nm; MS: mass range 150-750 amu; positive ion electrospray ionization.

Preparative HPLC were carried out with one of the two conditions listed below:
1) Condition 1: GILSON Preparative HPLC System (Gilson Incorporated, Middleton, WI, USA); Column: SHISEIDO CAPCELL PAK, MG (Shiseido Co., Ltd., Japan); C18, 20 mm×250 mm, 5 m; Mobile phase: Water+0.1% trifluoroacetic acid; ACN+0.1% trifluoroacetic acid; Method: 15 minutes gradient elution; Initial organic: 10%; Final organic: 80%; UV1: 240; UV2: 230; Flow: 15 ml/min.
2) Condition 2: GILSON Preparative HPLC System; Column: SunFire® Prep C18 OBD 5 μm, 19 mm×150 mm (Waters Corporation, Milford, MA USA); Mobile phase: Water+0.1% trifluoroacetic acid; ACN+0.1% trifluoroacetic acid; Method: 20 minutes gradient elution; Initial organic: 10%; Final organic: 80%; UV1: 220; UV2: 254; Flow: 15 ml/min.
3) Condition 3: GILSON Preparative HPLC System, GX-281, Column: Welch ultimate XB-C18, 21.2 mm×250 mm, 5 μm (Welch Materials, Inc., West Haven, CT USA), MeCN/H₂O with 0.05% TFA or 0.1% HCOOH.

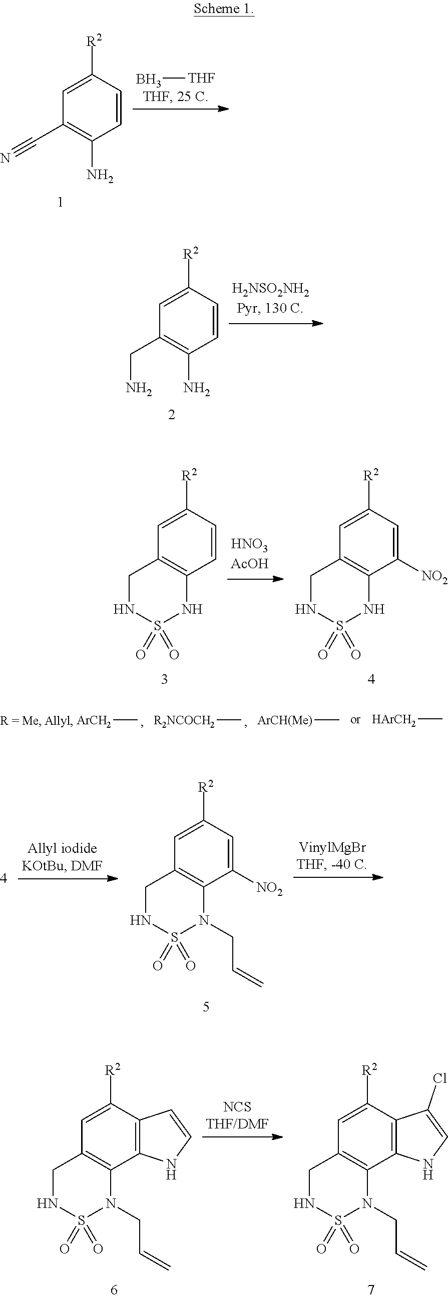

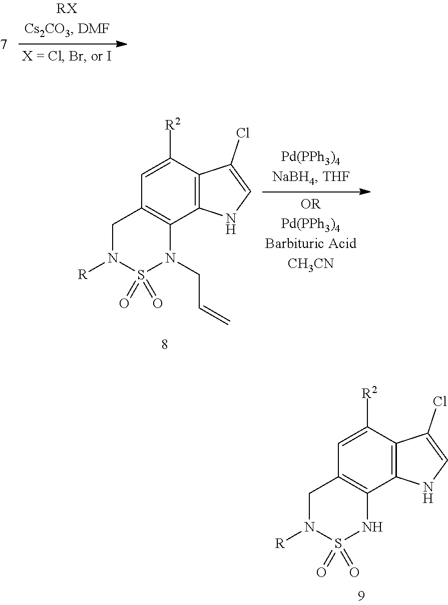

Scheme 1 illustrates a synthetic sequence for the preparation of cyclic sulfamide derivatives such as 5, 6, 7, and 9 from ortho cyano aniline derivatives such as 1, 5, 6, and 7 serve as useful intermediates in the preparation of myriad tricyclic sulfamide compounds. Compounds such as the tricyclic sulfamide 9 represent the culmination of the synthetic sequence and take advantage of intermediate 7 and reactivity with activated alkyl halides (methyl iodide, allyl iodide, (hetero)aryl benzylic halides, etc.). The sequence starts from ortho cyano aniline 1 or diamine 2. If the the sequence begins with ortho cyano aniline 1, reduction to diamine 2 is facilitated by treatment with borane tetrahydrofuran complex. Formation of the cyclic sulfamide 3 is achieved by heating diamine 2 with an excess of sulfamide in pyridine. Nitration of 3 in a mixture of acetic acid and nitric acid affords the ortho nitro aniline derivative 4. Allylation of the aniline nitrogen in ortho nitro aniline derivative 4 to afford allyl protected 5 is accomplished by pre-treatment with stoichiometric potassium t-butoxide followed by quenching with allyl iodide. Bartoli reaction by treatment of allyl protected 5 with vinyl Grignard results in formation of indole 6. Indole 6 is chlorinated by treatment with NCS to produce chloro indole 7. Chloro indole 7 is selectively functionalized at the sulfamide nitrogen to form alkylated intermediate 8 by treatment with activated alkyl halides in the presence of cesium carbonate. Allyl group removal from the alkylated intermediate 8 to form tricyclic sulfamide 9 is achieved by two possible palladium catalyzed deprotection methods: (1) treatment with palladium tetrakis and sodium borohydride or (2) treatment with palladium tetrakis and barbituric acid.

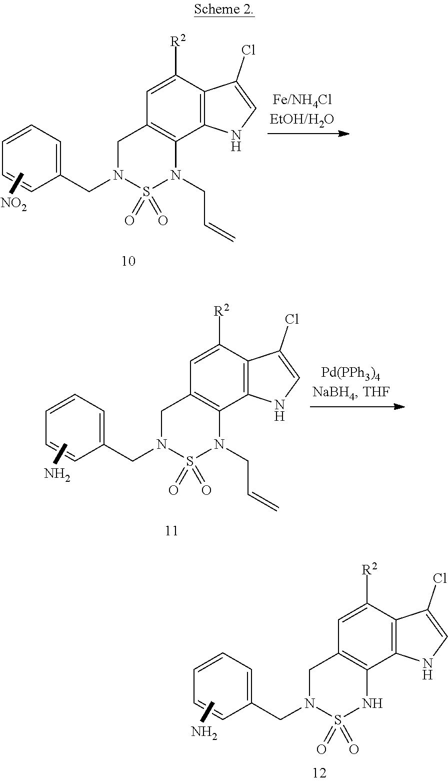

Scheme 2 illustrates a synthetic sequence for the synthesis of tricyclic sulfamides bearing a benzylic group with aniline functionality such as 12 from nitrobenzylic compounds such as 10. The nitro group of 10 is reduced with elemental iron to afford the aniline derivative 11. The aniline 11 is deallylated by treatment with palladium tetrakis and sodium borohydride to afford tricyclic sulfamide 12.

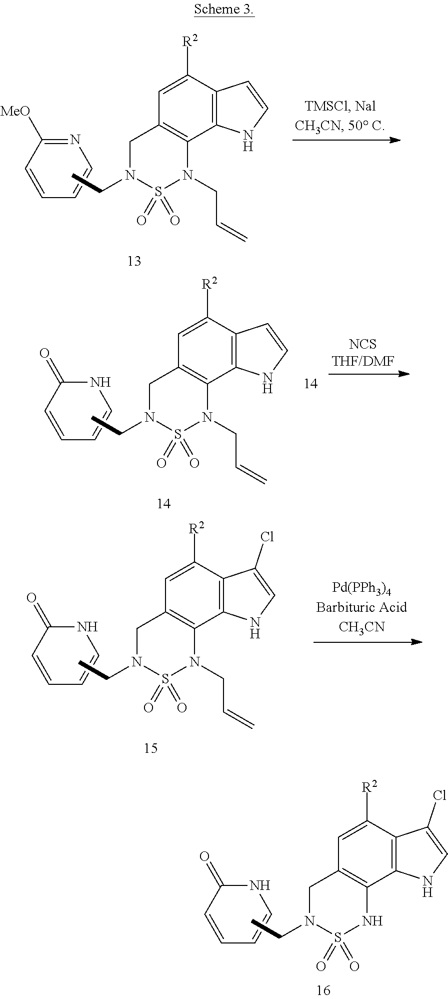

Scheme 3 illustrates a synthetic sequence for the preparation of tricyclic sulfamides bearing a pyridone group such as 16 from methoxypyridine derivatives such as 13. Deprotection of methoxy pyridine 13 with a mixture of sodium iodide and TMSCl results in pyridone 14 which has lost chlorination at the indole. Rechlorination of 14 is possible by treatment with NCS and affords the chloroindole 15. The allyl group of 15 can be removed by treatment with palladium tetrakis and barbituric acid to give tricyclic sulfamide 16.

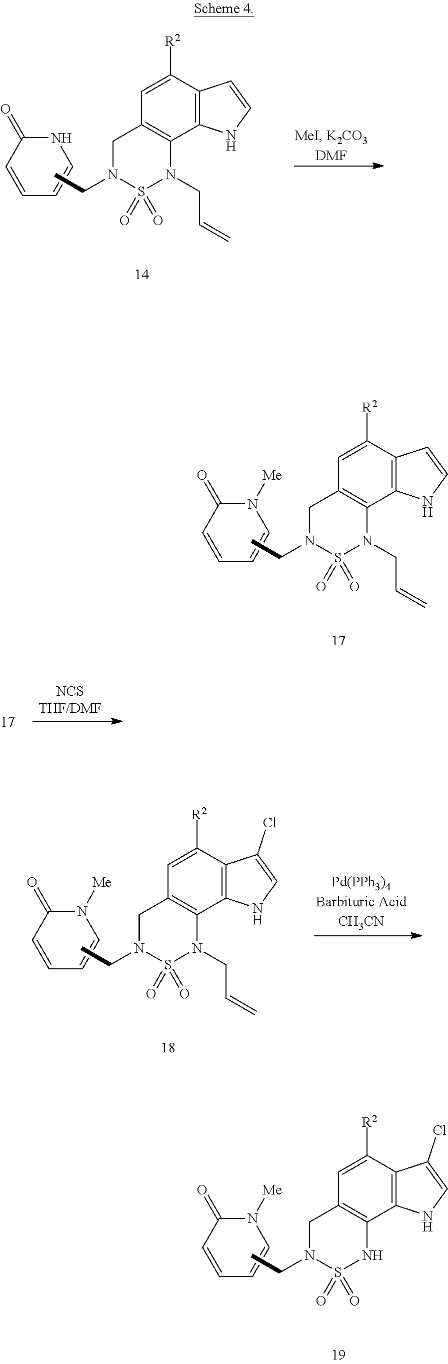

Scheme 4 illustrates a synthetic sequence for the preparation of tricyclic sulfamides bearing a methylated pyridone such as 19 from pyridone derivatives such as 14. Pyridone 14 is methylated by treatment with iodomethane to give methyl pyridone 17. Methyl pyridone 17 is chlorinated by the action of NCS to give chloroindole 18. Chloroindole 18 is deallylated by treatment with palladium tetrakis and barbituric acid to give tricyclic sulfamide 19.

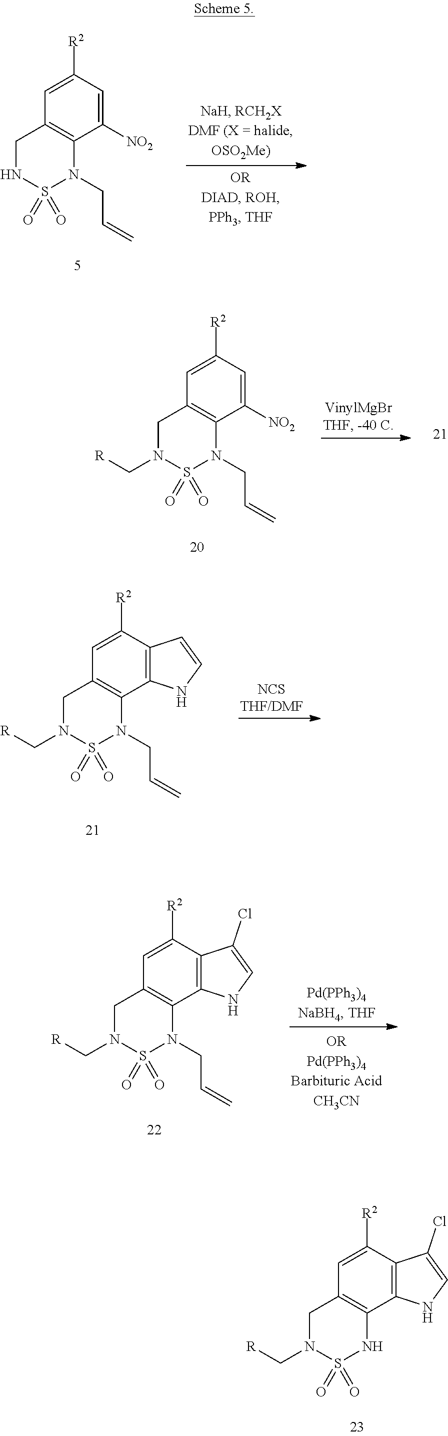

Scheme 5 illustrates a synthetic sequence for the preparation of tricyclic sulfamides such as 23 from cyclic sulfamides such as 5. Sulfamide 5 is alkylated to afford functionalized sulfamide 20 by one of two possible methods: (1) treatment with an alkyl halide or pseudohalide in the presence of sodium hydride or (2) Mitsunobu reaction with the appropriate alcohol. Functionalized sulfamide 20 is cyclized to indole 21 by the Bartoli reaction following treatment with vinyl Grignard. Indole 21 is chlorinated with NCS to afford the chloroindole 22. The chloroindole 22 is deallylated to give tricyclic sulfamide 23 by one of two possible methods: (1) treatment with palladium tetrakis and sodium borohydride or (2) treatment with palladium tetrakis and barbituric acid.

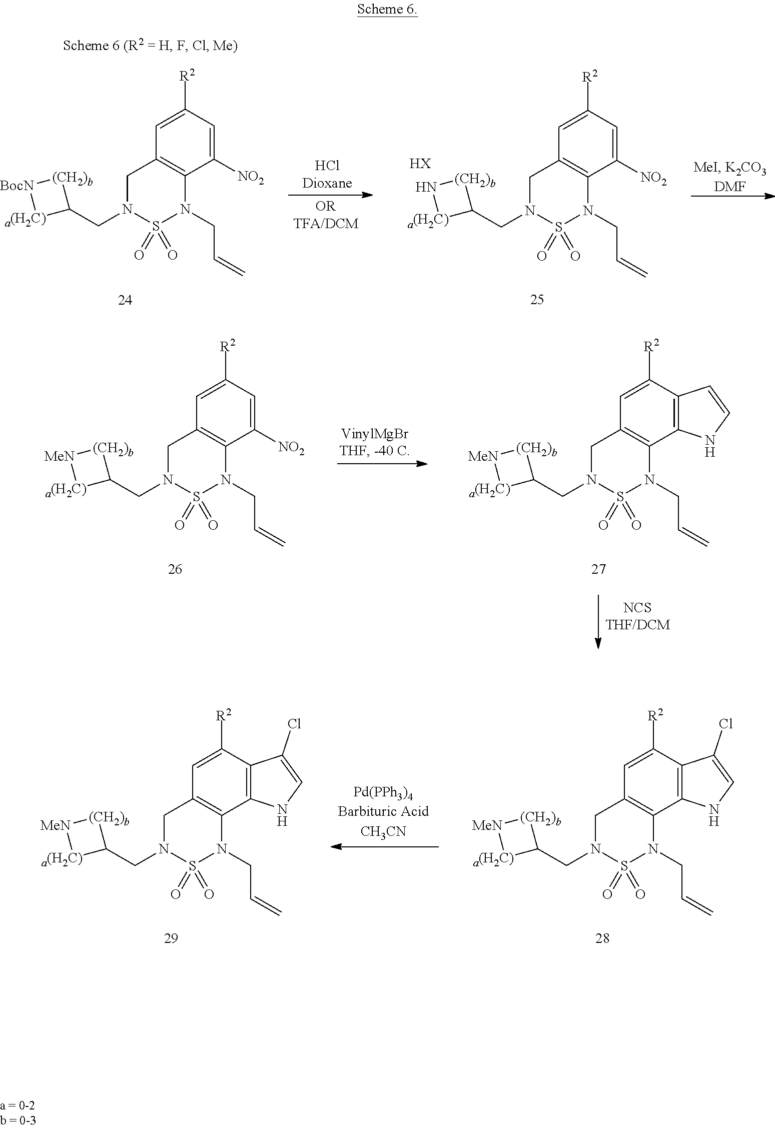

Scheme 6 illustrates a synthetic sequence for the syntheses of tricyclic sulfamides such as 29 bearing an tertiary amine. Sulfamide 24 loses its BOC group upon treatment with HCl or TFA to afford salt 25. Salt 25 is methylated to give tertiary amine 26 following liberation of the free base with potassium carbonate and treatment with iodomethane. Tertiary amine 26 is cyclized to indole 27 by Bartoli reaction following treatment with vinyl Grignard. Indole 28 is chlorinated with NCS to afford the chloroindole 28. Chloroindole 28 is deallylated by treatment with palladium tetrakis and barbituric acid to give tricyclic sulfamide 29.

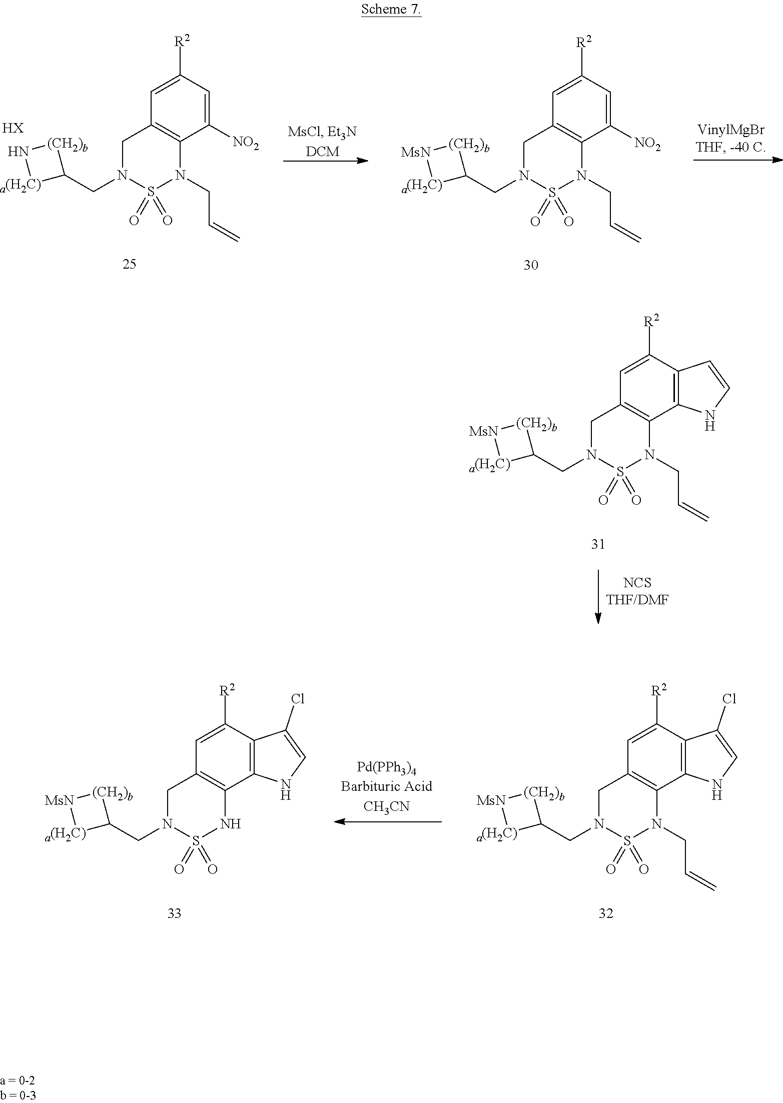

Scheme 7 illustrates a synthetic sequence for the preparation of tricyclic sulfamides such as 33 bearing a sulfonamide from amine salts such as 25. Salt 25 is converted to sulfonamide 30 via treatment with methanesulfonyl chloride. Sulfonamide 30 is cyclized to indole 31 by Bartoli reaction following treatment with vinyl Grignard. Indole 31 is chlorinated with NCS to afford the chloroindole 32. Chloroindole 32 is deallylated by treatment with palladium tetrakis and barbituric acid to give tricyclic sulfamide 33.

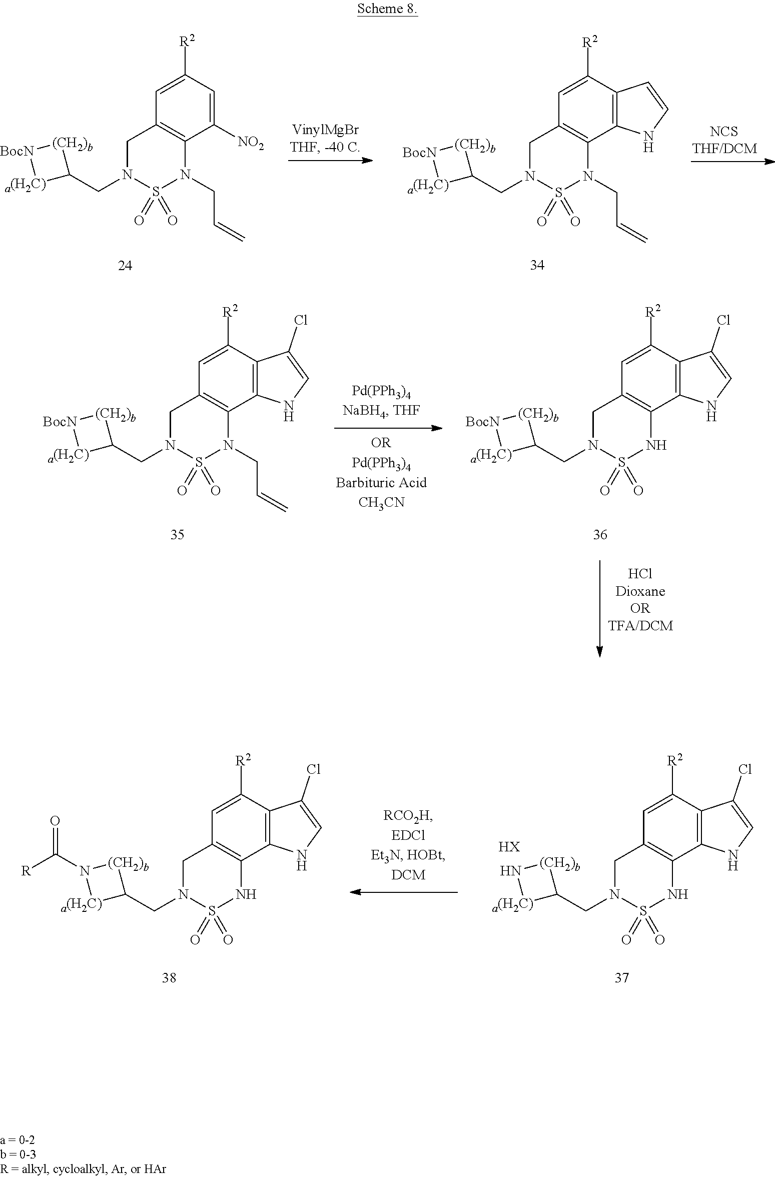

Scheme 8 illustrates a synthetic sequence for the synthesis of tricyclic sulfamides bearing an amide such as 38 from carbamates such as 24. Carbamate 24 is cyclized to indole 34 by Bartoli reaction following treatment with vinyl Grignard. Indole 34 is chlorinated with NCS to afford the chloroindole 35. Chloroindole 35 is deallylated to sulfamide 36 by one of two possible methods: (1) treatment with palladium tetrakis and sodium borohydride or (2) treatment with palladium tetrakis and barbituric acid. Sulfamide 36 loses its BOC group upon treatment with HCl or TFA to afford salt 37. Amide formation from salt 37 to afford tricyclic sulfamide 38 is achieved under standard peptide coupling conditions utilizing EDCI as the coupling agent.

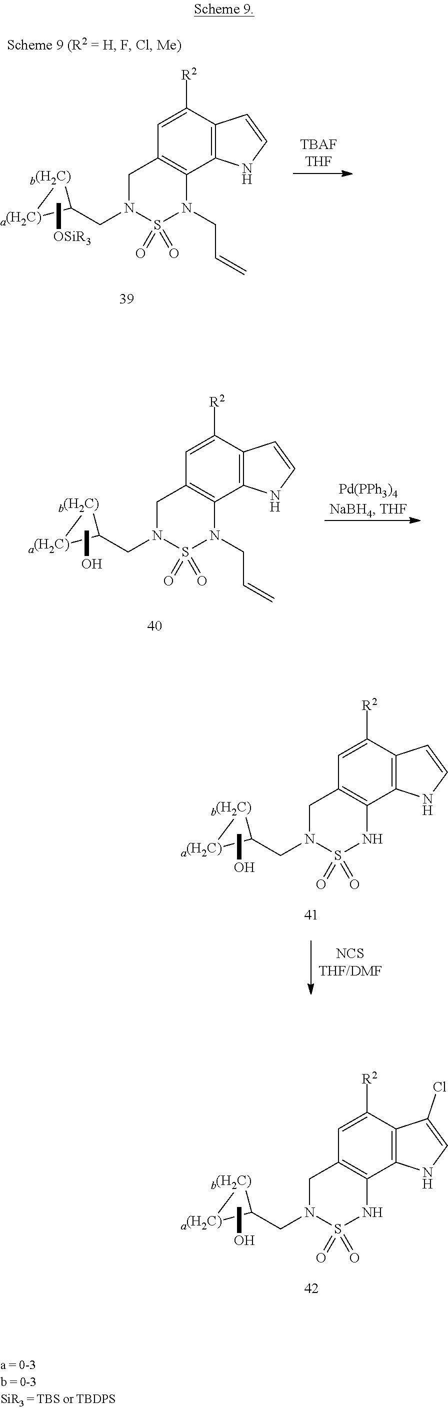

Scheme 9 illustrates a synthetic sequence for the syntheses of tricyclic sulfamides bearing an alcohol such as 42 from silylated alcohols such as 39. Silylated alcohol 39 is converted to substituted alcohol 40 via silyl deprotection with TBAF. The alcohol 40 is deallylated by treatment with palladium tetrakis and sodium borohydride to afford sulfamide 41. Sulfamide 41 is chlorinated with NCS to afford the tricyclic sulfamide derivative 42.

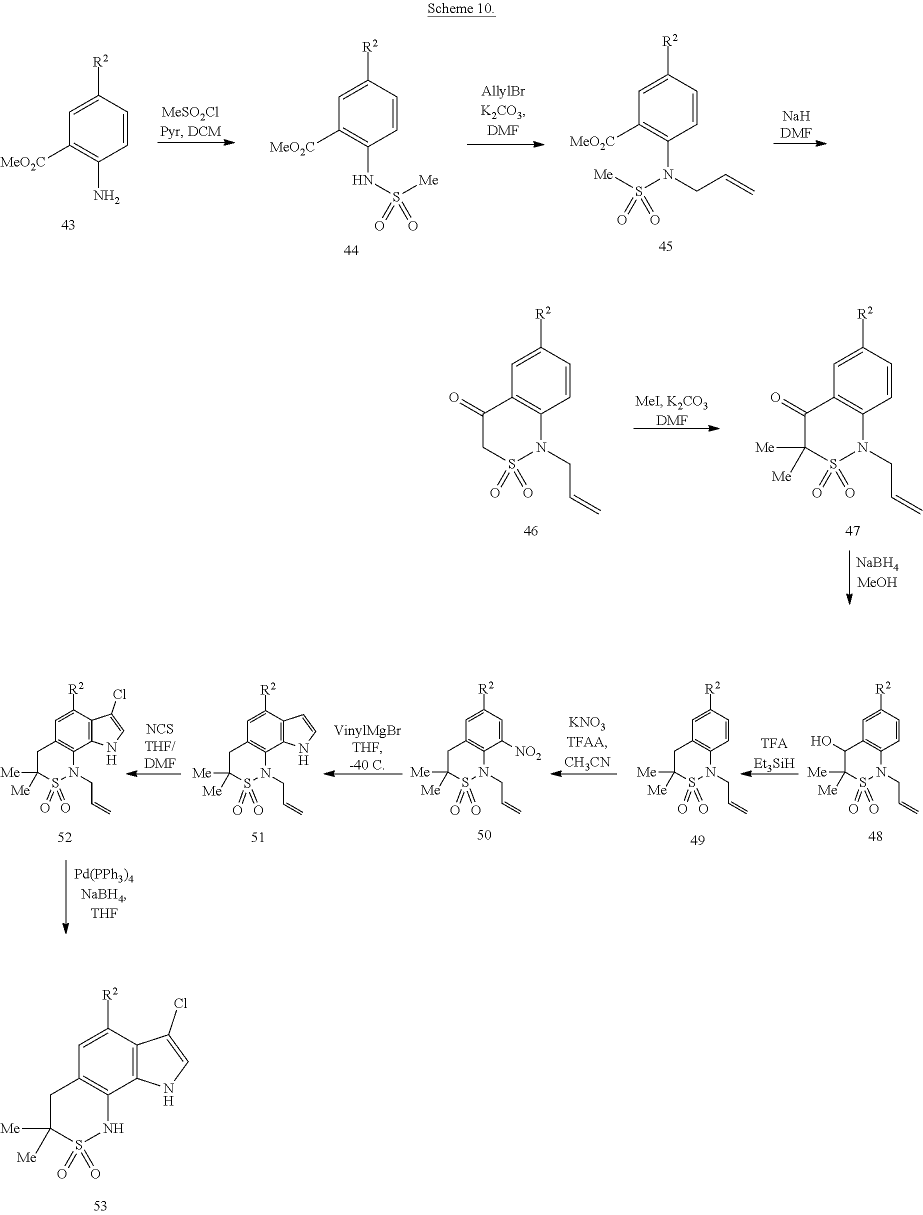

Scheme 10 illustrates a synthetic sequence for the synthesis of tricyclic sultams such as 53 from anthranilate derivatives such as 43. Anthranilate 43 is reacted with methanesulfonyl chloride to yield sulfonamide 44. Sulfonamide 44 is allylated by treatment with allyl bromide in the presence of potassium carbonate to afford allylated sulfonamide 45. Allylated sulfonamide 45 is cyclized to J-keto sultam 46 by treatment with sodium hydride. The J-keto sultam 46 is bis-alkylated with iodomethane in the presence of potassium carbonate to give dimethylated sultam 47. Reduction of dimethylated sultam 47 with sodium borohydride results in alcohol 48. The alcohol function in 48 is then removed by treatment with triethylsilane and TFA to provide sultam 49. Sultam 49 is nitrated by activating potassium nitrate with TFAA to produce nitro sultam 50. Nitro sultam 50 is cyclized to indole 51 by Bartoli reaction following treatment with vinyl Grignard. Indole 51 is chlorinated with NCS to afford the chloroindole 52. The chloroindole 52 is deallylated by treatment with palladium tetrakis and sodium borohydride to afford tricyclic sultam 53.

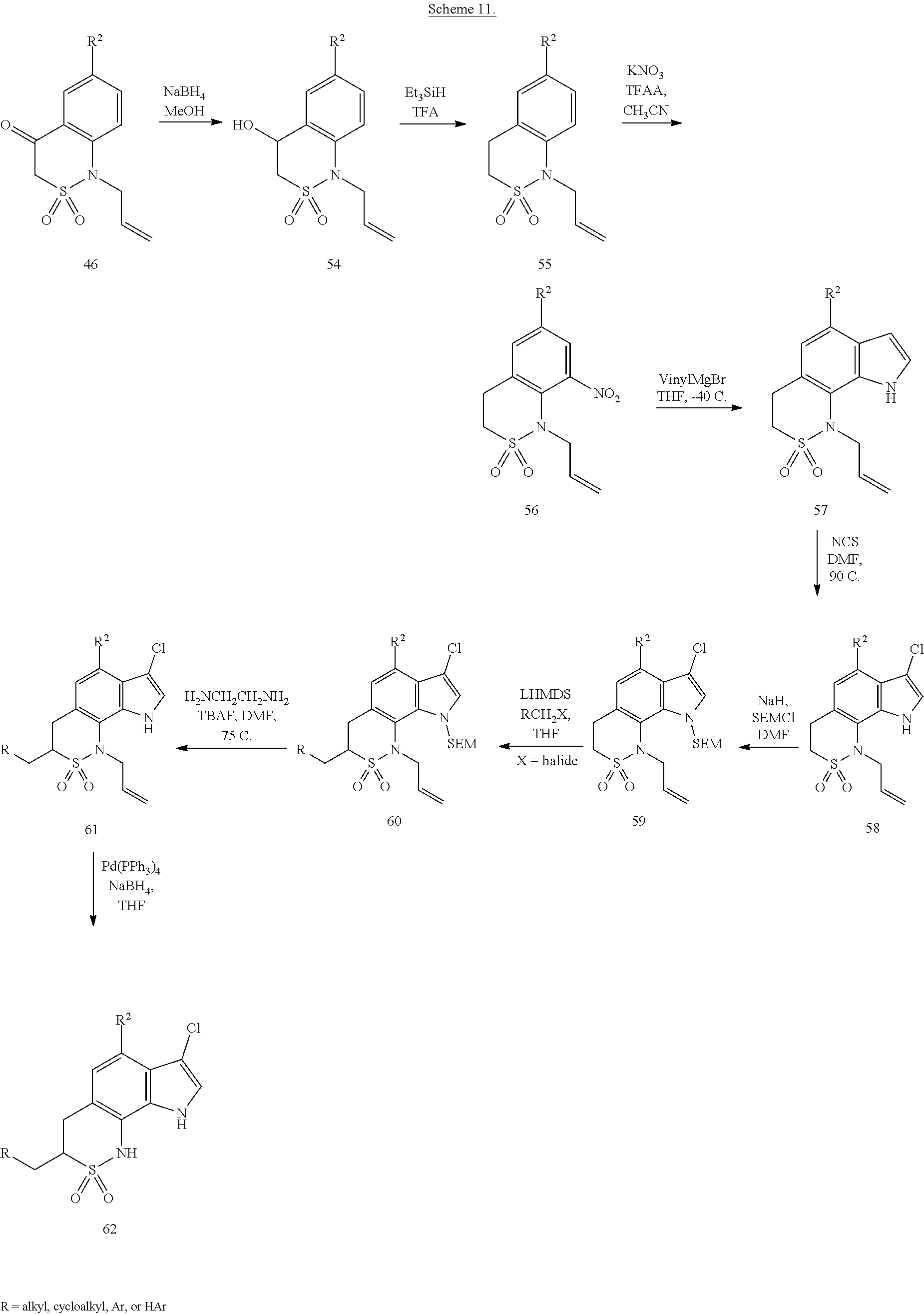

Scheme 11 illustrates a synthetic sequence for the preparation of tricyclic sultam derivatives such as 62 from-keto sultam 46. Reduction of 3-keto sultam 46 with sodium borohydride affords alcohol 54. The alcohol function in 54 is then removed by treatment with triethylsilane and TFA to provide sultam 55. Sultam 55 is nitrated by activating potassium nitrate with TFAA to produce nitro sultam 56. Nitro sultam 56 is cyclized to indole 57 by Bartoli reaction following treatment with vinyl Grignard. Indole 57 is chlorinated with NCS to afford the chloro indole 58. The chloroindole 58 is protected by treatment with SEMCl in the presence of sodium hydride to yield the SEM protected indole 59. The SEM protected indole 59 is alkylated to form sultam 60 by treatment with LHMDS and quenching with the appropriate alkyl halide. The SEM group of sultam 60 is removed by the action of TBAF in the presence 1,2-diaminoethane to produce deprotected indole 61. The deprotected indole 61 is deallylated by treatment with palladium tetrakis and sodium borohydride to afford tricyclic sultam 62.

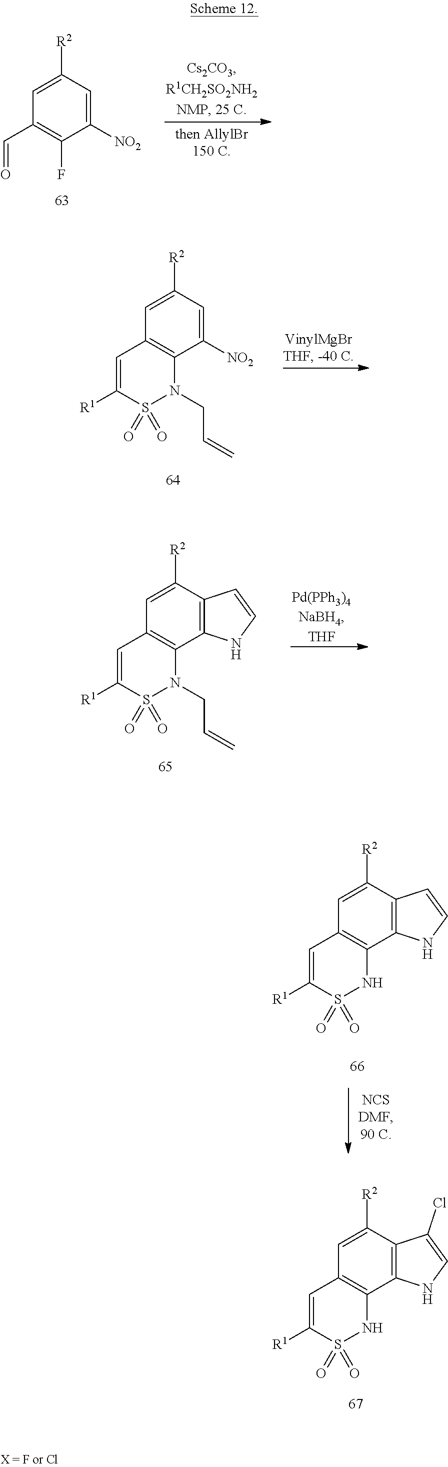

Scheme 12 illustrates a synthetic sequence for the preparation of unsaturated tricyclic sultams 67 from nitro benzaldehydes such as 63. Transformation of the nitro benzaldehyde 63 into sultam 64 is achieved by a 2-step one pot sequence. First, the sulfonamide component is introduced by displacement of the fluoro group. Then allyl bromide is introduced to alkylate the sulfonamide. Finally, the resulting mixture is heated to high temperature to induce hydroxyl group elimination and olefin formation. Sultam 64 is cyclized to indole 65 by Bartoli reaction following treatment with vinyl Grignard. Indole 65 is deallylated by treatment with palladium tetrakis and sodium borohydride to afford deallylated indole 66. The deallylated indole 66 is chlorinated with NCS to afford the unsaturated tricyclic sultam 67.

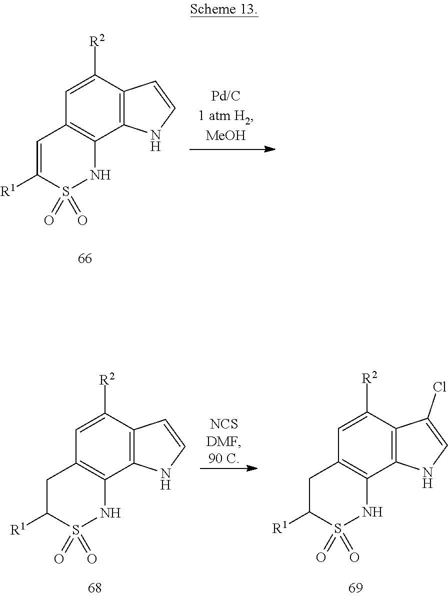

Scheme 13, in combination with Scheme 12, represents an alternative preparation of tricyclic sultam derivatives such as 69 from the unsaturated tricyclic sultam 66. The unsaturated tricyclic sultam 66 is hydrogenated under an atmosphere of hydrogen gas in the presence of palladium on carbon to afford saturated tricyclic sultam 68. The saturated tricyclic sultam 68 is chlorinated with NCS to afford the tricyclic sultam 69.

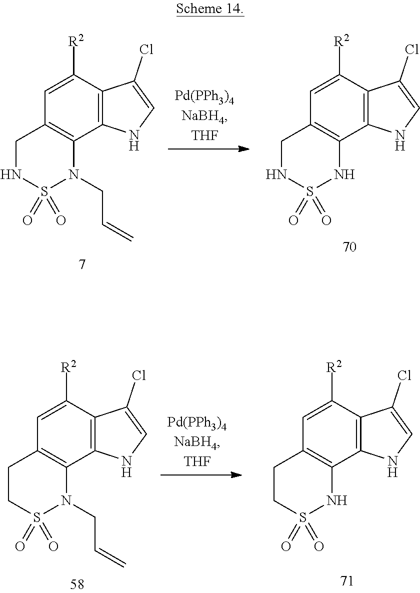

Scheme 14 illustrates a synthetic sequence for the preparation of unfunctionalized tricyclic sulfamide or sultam derivatives such as 70 and 71 from allylated precursors such as 5 and 58. The allylated precursors 5 and 58 are deallylated by treatment with palladium tetrakis and sodium borohydride to afford tricyclic sulfamide and sultam derivatives 70 and 71.

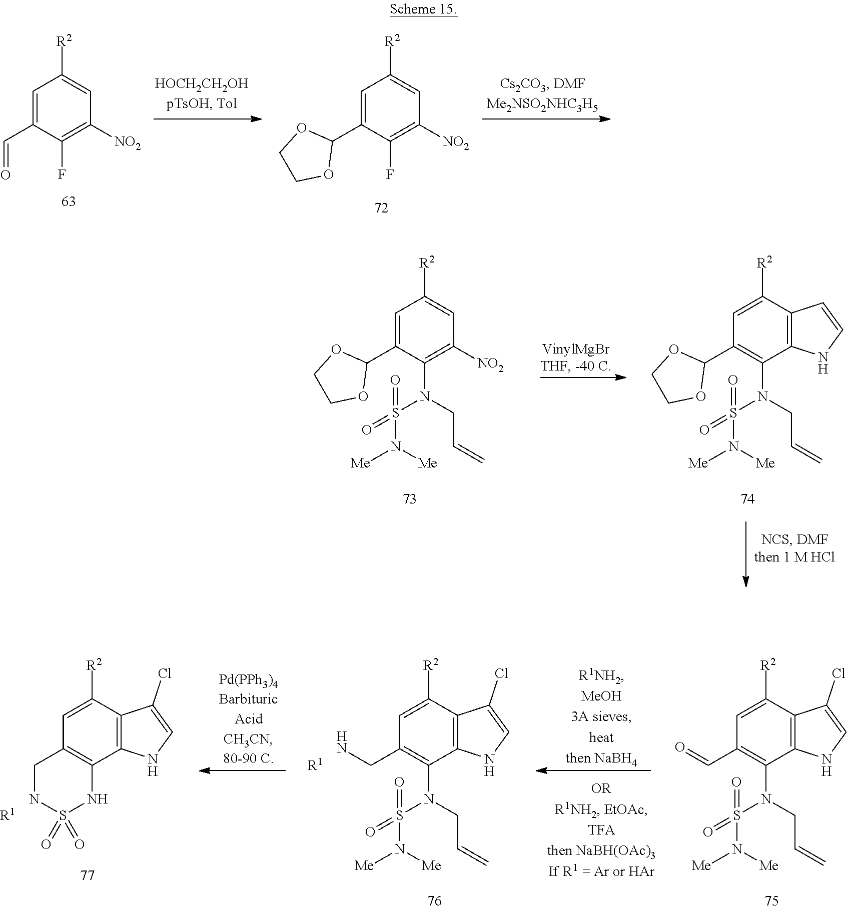

Scheme 15 illustrates an alternate synthetic sequence for the preparation of tricyclic sulfamide derivatives such as 77 from nitro benzaldehydes such as 63. Acetalization of nitro benzaldehyde 63 is accomplished under dehydrating conditions with ethylene glycol to afford acetal 72. Acetal 72 is aminated by treatment with a trialkylated sulfamide to afford sulfamide 73. Sulfamide 73 is cyclized to indole 74 by Bartoli reaction following treatment with vinyl Grignard. Transformation of indole 74 to aldehyde 75 is achieved by a sequential one pot sequence involving chlorination with NCS and then acetal group removal by treatment with aqueous hydrochloric acid. Aldehyde 75 serves as a versatile intermediate for the late stage introduction of the R¹ component by reductive amination to make amine 76. Deallylation of amine 76 by treatment with palladium tetrakis and barbituric acid also results in concomitant cyclization to the desired tricyclic sulfamide 77.

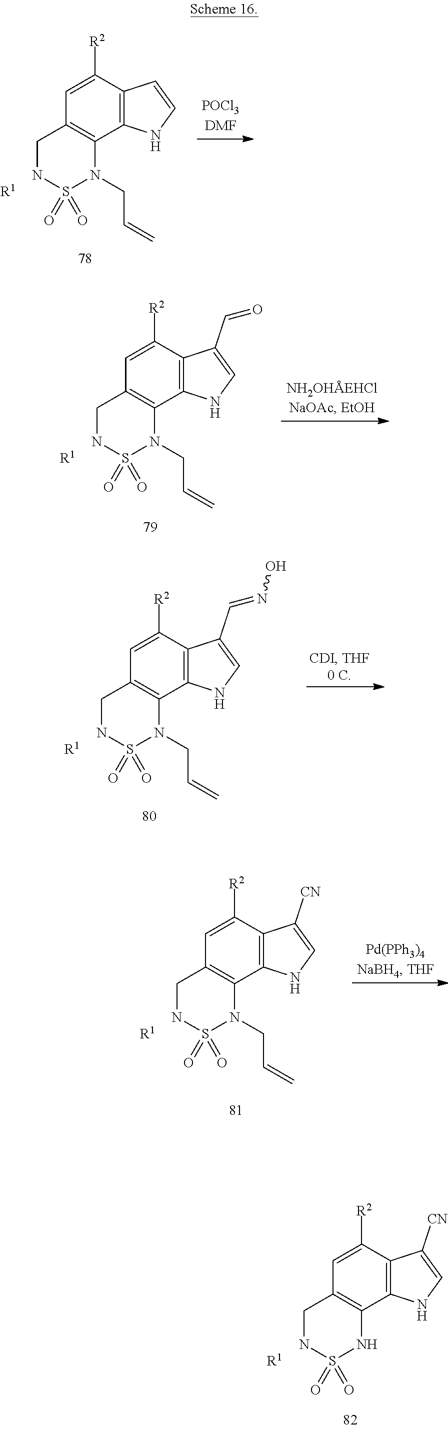

Scheme 16 illustrates a synthetic sequence for the preparation of cyanated tricyclic sulfamides such as 82 from indole derivatives such as 78. The indole is transformed to aldehyde 79 by the action of the Vilsmeier salt generated from POCl₃ added to DMF. Treatment of aldehyde 79 with hydroxylamine results in formation of oxime 80. CDI initiates the dehydration of oxime 80 to afford nitrile 81. The nitrile 81 is deallylated by treatment with palladium tetrakis and sodium borohydride to afford tricyclic sulfamide derivative 82.

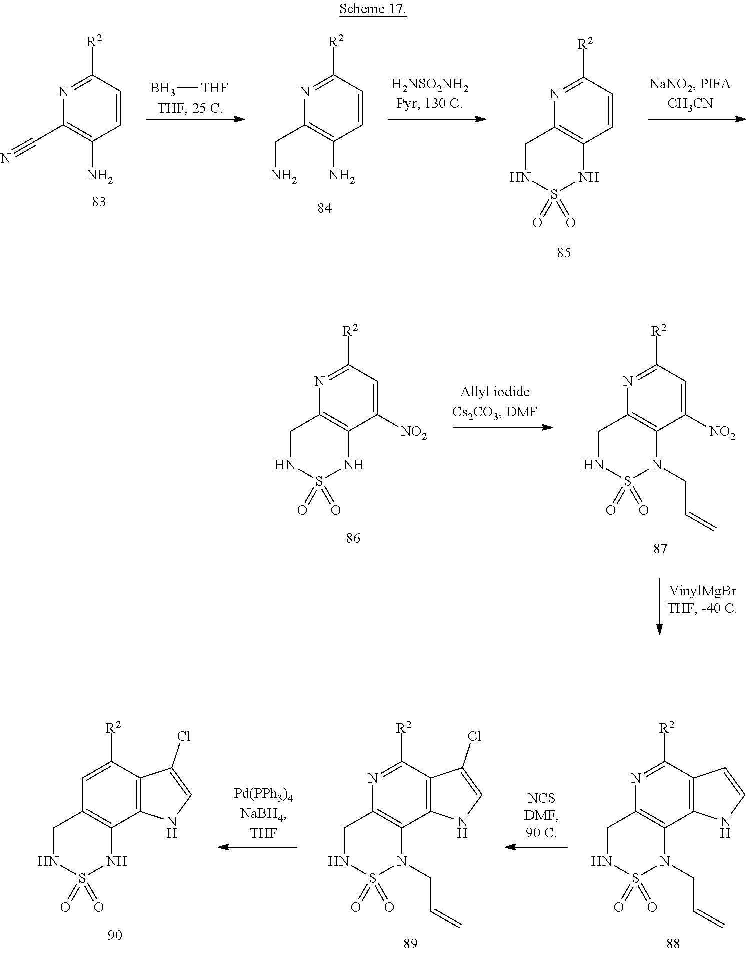

Scheme 17 illustrates a synthetic sequence for the preparation of pyridine containing tricyclic sulfamides such as 90 from 2-cyano pyridine derivatives such as 83. Reduction of 2-cyano pyridine 83 to afford diamine 84 is facilitated by treatment with borane tetrahydrofuran complex. Formation of the cyclic sulfamide 85 is achieved by heating diamine 84 with an excess of sulfamide in pyridine. Nitration of sulfamide 85 is achieved by treatment with sodium nitrite and PIFA to yield nitro pyridine 86. Allylation of the aniline nitrogen in nitro pyridine 86 to afford allyl protected 87 is accomplished by treatment with cesium carbonate and allyl iodide.

Bartoli reaction by treatment of allyl protected 87 with vinyl Grignard results in formation of indole 88. Indole 88 is chlorinated by treatment with NCS to produce chloro indole 89. Allyl group removal from chloro indole 89 to form pyridine containing tricyclic sulfamide 90 is achieved by treatment with palladium tetrakis and sodium borohydride.

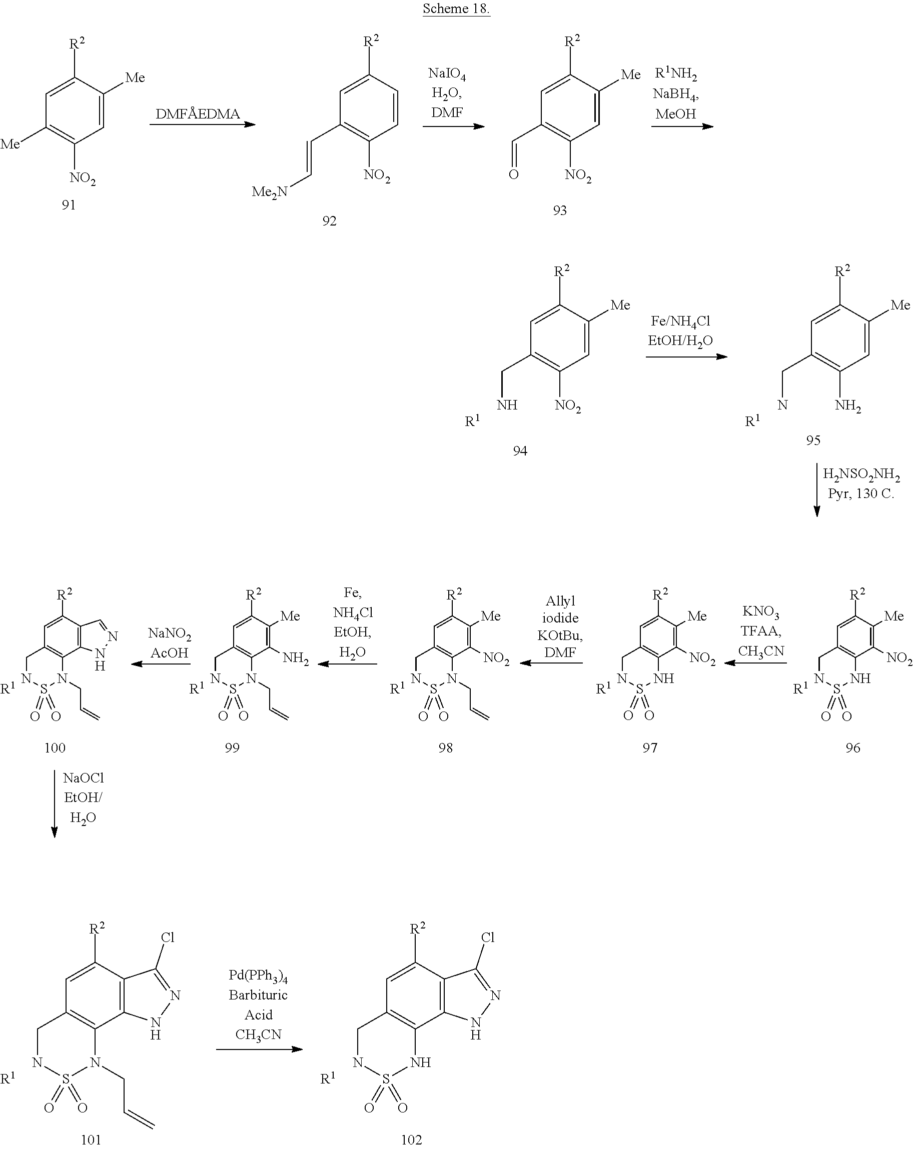

Scheme 18 illustrates a synthetic sequence for the preparation of indazole containing tricyclic sulfamides such as 101 from nitro xylene derivatives such as 91. Treatment of nitro xylene 91 with DMF dimethyl acetal results in formation of the enamine 92. Enamine 92 is oxidized by the action of sodium periodate to afford aldehyde 93. Reductive amination of aldehyde 93 with an amine yields nitro aniline 94. Nitro aniline 94 is reduced by the action of elemental iron to diamine 95. Formation of the cyclic sulfamide 96 is achieved by heating diamine 95 with an excess of sulfamide in pyridine. Cyclic sulfamide 96 is nitrated by activating potassium nitrate with TFAA to produce nitro sultam 97. Nitro sulfamide 97 is allylated by pre-treatment with stoichiometric potassium t-butoxide followed by quenching with allyl iodide to give allyl protected 98. The allyl protected 98 is reduced to amino sulfamide 99 by the action of elemental iron. Amino sulfamide 99 is cyclized to the indazole 100 by diazotization with sodium nitrite. Indazole 100 is chlorinated by use of bleach to afford chloro indazole 101. Allyl group removal from chloro indazole 100 to form indazole containing tricyclic sulfamide 102 is achieved by treatment with palladium tetrakis and sodium borohydride.

### Preparatory Examples 1, 2, and 3

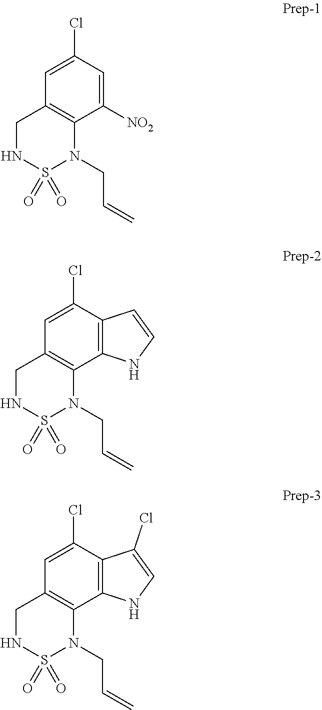 [SMILES: C=CCN1c2c(cc(Cl)cc2[N+](=O)[O-])CNS1(=O)=O ; C=CCN1c2c(cc(Cl)c3cc[nH]c23)CNS1(=O)=O ; C=CCN1c2c(cc(Cl)c3c(Cl)c[nH]c23)CNS1(=O)=O]

### 1-allyl-6-chloro-8-nitro-3,4-dihydro-1H-benzo[c][1,2,6]thiadiazine 2,2-dioxide (Prep-1)

### 1-allyl-6-chloro-1,3,4,9-tetrahydro-[1,2,6]thiadiazino[4,3-g]indole 2,2-dioxide (Prep-2), and

### 1-allyl-6,7-dichloro-1,3,4,9-tetrahydro-[1,2,6]thiadiazino[4,3-g]indole 2,2-dioxide (Prep-3)

Preparatory Examples 1, 2 and 3 were prepared in an analogous manner to that outlined in Steps 1 through 6 of Scheme 1.

### Step 1. 2-(aminomethyl)-4-chloroaniline

To a solution of 2-amino-5-chlorobenzonitrile (20 g, 131.9 mmol) in dry THF (200 ml) at 0° C. under argon atmosphere was slowly added borane (158 ml, 158.3 mmol, ˜1.0 M in THF). The mixture was stirred at 0° C. for 10 min and 48 h at room temperature. The reaction mixture was cooled to 0° C. and quenched by the addition of HCl in MeOH (4 M). The precipitate was filtered and collected, and then treated with saturated aqueous ammonia solution. The resulting suspension was extracted with EtOAc (3×300 mL). The combined organic layers were dried over sodium sulfate. EtOAc was completely removed under reduced pressure to give 2-(aminomethyl)-4-chloroaniline as a solid. ESI MS [M-16]⁺ for C₇H₉ClN₂, calcd 140.05, found 140.1.

### Step 2. 6-chloro-3,4-dihydro-1H-benzo[c][1,2,6]thiadiazine 2,2-dioxide

To a stirred solution of 2-(aminomethyl)-4-chloroaniline (12.8 g, 81.73 mmol) in pyridine (130 mL) was added sulfuric diamide (23.5 g, 245.2 mmol) and the resulting mixture was heated to 130° C. for 5 h. After cooling, the reaction mixture was concentrated in vacuo. The residue was dissolved in DCM (200 ml) and the solution was washed with water (100 mL), dried over Na₂SO₄ and concentrated in vacuo. The resulting residue was crystallized from toluene to give 6-chloro-3,4-dihydro-1H-benzo[c][1,2,6]thiadiazine 2,2-dioxide as a solid. MS=216.9 (M−1).

### Step 3. 6-chloro-8-nitro-3,4-dihydro-1H-benzo[c][1,2,6]thiadiazine 2,2-dioxide

To a solution of 6-chloro-3,4-dihydro-1H-benzo[c][1,2,6]thiadiazine 2,2-dioxide (17 g, 77.75 mmol) in acetic acid (170 mL) was added fuming nitric acid (50 mL) dropwise at 0° C. After complete conversion of starting material, the mixture was concentrated in vacuo. The residue was poured into ice-water (100 mL) and treated with Na₂CO₃ to adjust pH to 6, The precipitate was filtered and dried to give 6-chloro-8-nitro-3,4-dihydro-1H-benzo[c][1,2,6]thiadiazine 2,2-dioxide as brown solid. MS=262.0 (M−1).

### Step 4. 1-allyl-6-chloro-8-nitro-3,4-dihydro-1H-benzo[c][1,2,6]thiadiazine 2,2-dioxide (Preparatory Example 1)

A solid mixture 6-chloro-8-nitro-3,4-dihydro-1H-benzo[c][1,2,6]thiadiazine 2,2-dioxide (24.8 g, 75.84 mmol) and potassium 2-methylpropan-2-olate (9.36 g, 83.44 mmol) was suspended in DMF (200 mL) and stirred at 25° C. for 15 min. The dark red solution was treated with allyl iodide (14.0 g, 83.44 mmol) and left to stir at room temperature until complete by LCMS. The reaction mixture was poured into 1000 mL of water and extracted with EtOAc (3×450 mL). The combined organics were washed with 400 mL of brine, dried over MgSO₄, filtered and concentrated to dryness. The residue was purified by column chromatography (silica gel, EtOAc/PE=1/1) to give 1-allyl-6-chloro-8-nitro-3,4-dihydro-1H-benzo[c][1,2,6]thiadiazine 2,2-dioxide (Prep-1). MS=302.0 (M−1).

### Step 5. 1-allyl-6-chloro-1,3,4,9-tetrahydro-[1,2,6]thiadiazino[4,3-g]indole 2,2-dioxide (Preparatory Example 2)

To a solution of 1-allyl-6-chloro-8-nitro-3,4-dihydro-1H-benzo[c][1,2,6]thiadiazine 2,2-dioxide (Prep-1, 12.8 g, 42.14 mmol) in dry THF (130 mL) was added vinyl magnesium bromide (210 mL, 210 mmol, ˜1.0 M in THF) at −78° C. Then the reaction was stirred for two hours at −40° C. before being quenched with 80 ml saturated aqueous NH₄Cl solution. The product was extracted with EtOAc (3×100 mL). The combined organic layer was dried over MgSO₄. After filtration and concentration, the crude product was purified by column chromatography (silica gel, EtOAc/PE=1/4 to 1/2) to afford 1-allyl-6-chloro-1,3,4,9-tetrahydro-[1,2,6]thiadiazino[4,3-g]indole 2,2-dioxide (Prep-2). MS=296.0 (M−1).

### Step 6. 1-allyl-6,7-dichloro-1,3,4,9-tetrahydro-[1,2,6]thiadiazino[4,3-g]indole 2,2-dioxide (Preparatory Example 3)

To a solution of 1-allyl-6-chloro-1,3,4,9-tetrahydro-[1,2,6]thiadiazino[4,3-g]indole 2,2-dioxide (Prep-2, 3.2 g, 10.75 mmol) in THF (300 mL) and DMF (6 mL) was added NCS (1.58 g, 11.84 mmol) at 0° C. The reaction was stirred at rt overnight. The mixture was treated with saturated aqueous Na₂SO₃ solution (100 mL), stirred for 10 min, and extracted with EtOAc (3×100 mL). The organic layer was washed with brine (50 mL) and concentrated after being dried over Na₂SO₄. 1-allyl-6,7-dichloro-1,3,4,9-tetrahydro-[1,2,6]thiadiazino[4,3-g]indole 2,2-dioxide (Prep-3) was obtained by column chromatography (silica gel, EtOAc/PE=1/4 to 1/2). MS=330.0 (M−1).

### Examples 1 and 2

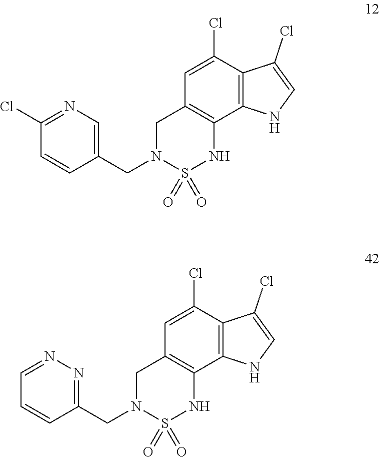 [SMILES: O=S1(=O)Nc2c(cc(Cl)c3c(Cl)c[nH]c23)CN1Cc1ccc(Cl)nc1 ; O=S1(=O)Nc2c(cc(Cl)c3c(Cl)c[nH]c23)CN1Cc1cccnn1]

### 6,7-dichloro-3-((6-chloropyridin-3-yl)methyl)-1,3,4,9-tetrahydro-[1,2,6]thiadiazino[4,3-g]indole 2,2-dioxide (Compound 12) and 6,7-dichloro-3-(pyridazin-3-ylmethyl)-1,3,4,9-tetrahydro-[1,2,6]thiadiazino[4,3-g]indole 2,2-dioxide (Compound 42)

Compounds 12 and 42 were prepared in an analogous manner to that outlined in Steps 7 and 8 of Scheme 1.

### Step 7. 1-allyl-6,7-dichloro-3-((6-chloropyridin-3-yl)methyl)-1,3,4,9-tetrahydro-[1,2,6]thiadiazino[4,3-g]indole 2,2-dioxide

To a solution of 1-allyl-6,7-dichloro-1,3,4,9-tetrahydro-[1,2,6]thiadiazino[4,3-g]indole 2,2-dioxide (70.0 mg, 0.21 mmol) and Cs₂CO₃ (205.3 mg, 0.63 mmol) in DMF (5 mL) was added 2-chloro-5-(chloromethyl)pyridine (34.1 mg, 0.21 mmol). The mixture was stirred at rt for 16 h. Then H₂O was added to quench the reaction, and the product was extracted with EtOAc, dried over anhydrous Na₂SO₄, filtered, and the filtrate was concentrated and purified by Prep-TLC (EtOAc:PE=1:2) to afford 1-allyl-6,7-dichloro-3-((6-chloropyridin-3-yl)methyl)-1,3,4,9-tetrahydro-[1,2,6]thiadiazino[4,3-g]indole 2,2-dioxide.

### Step 8. 6,7-dichloro-3-((6-chloropyridin-3-yl)methyl)-1,3,4,9-tetrahydro-[1,2,6]thiadiazino[4,3-g]indole 2,2-dioxide (12)

Deprotection Method A: To a solution of 1-allyl-6,7-dichloro-3-((6-chloropyridin-3-yl)methyl)-1,3,4,9-tetrahydro-[1,2,6]thiadiazino[4,3-g]indole 2,2-dioxide (49.0 mg, 0.11 mmol) in THF (1 mL) was added NaBH₄ (2.0 equiv) and Pd(PPh₃)₄(0.05 equiv). The mixture was stirred at 25° C. overnight. Then aqueous saturated NH₄Cl solution (2 mL) was added to quench the reaction, and the product was extracted with EtOAc (3×5 mL), dried over anhydrous Na₂SO₄, filtered, and the filtrate concentrated. Purification by Prep-HPLC afforded 6,7-dichloro-3-((6-chloropyridin-3-yl)methyl)-1,3,4,9-tetrahydro-[1,2,6]thiadiazino[4,3-g]indole 2,2-dioxide (12) as the TFA salt. ¹H NMR (400 MHz, CD₃OD) δ 8.33 (s, 1H), 7.87 (d, J=8.8 Hz, 1H), 7.48 (d, J=8.4 Hz, 1H), 7.36 (s, 1H), 6.87 (s, 1H), 4.65 (s, 2H), 4.21 (s, 2H). MS=417.0 (M+1).

### Step 8. 6,7-dichloro-3-(pyridazin-3-ylmethyl)-1,3,4,9-tetrahydro-[1,2,6]thiadiazino[4,3-g]indole 2,2-dioxide (42)

The starting material 1-allyl-6,7-dichloro-3-(pyridazin-3-ylmethyl)-1,3,4,9-tetrahydro-[1,2,6]thiadiazino[4,3-g]indole 2,2-dioxide was prepared in an identical fashion to 1-allyl-6,7-dichloro-3-((6-chloropyridin-3-yl)methyl)-1,3,4,9-tetrahydro-[1,2,6]thiadiazino[4,3-g]indole 2,2-dioxide (step 7) using 3-(chloromethyl)pyridazine hydrochloride. Deprotection Method B: To a solution of 1-allyl-6,7-dichloro-3-(pyridazin-3-ylmethyl)-1,3,4,9-tetrahydro-[1,2,6]thiadiazino[4,3-g]indole 2,2-dioxide (41.0 mg, 0.097 mmol) in MeCN (1 mL) was added 1,3-dimethylbarbituric acid (150.9 mg, 0.97 mmol) and Pd(PPh₃)₄(0.05 eq). The mixture was stirred at 70° C. for 2 h. Then H₂O (20 mL) was added to quench the reaction, and the product was extracted with EtOAc (3×10 mL), dried over anhydrous Na₂SO₄, filtered, the filtrate was concentrated and purified by Prep-HPLC to afford 6,7-dichloro-3-(pyridazin-3-ylmethyl)-1,3,4,9-tetrahydro-[1,2,6]thiadiazino[4,3-g]indole 2,2-dioxide (42). ¹H NMR (400 MHz, CD₃OD): δ 9.13 (d, J=4.4 Hz, 1H), 7.95 (d, J=8.4 Hz, 1H), 7.77 (dd, J=8.4, 5.2 Hz, 1H), 7.33 (s, 1H), 6.84 (s, 1H), 4.71 (s, 2H), 4.48 (s, 2H). MS=384.0 (M+1).

TABLE 1
The following compounds were prepared using procedures similar to those
described for Examples 1 and 2 using the appropriate starting materials.

| | | | | Exact Mass |
|---|---|---|---|---|
| Comp. | | | Deprotection | [M + H]⁺ or |
| No. | Structure | Name | Method | [M − H]⁻ |
| 6 | 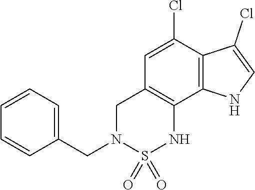 [SMILES: O=S1(=O)Nc2c(cc(Cl)c3c(Cl)c[nH]c23)CN1Cc1ccccc1] | 3-benzyl-6,7-dichloro- 1,3,4,9-tetrahydro- [1,2,6]thiadiazino[4,3- g]indole 2,2-dioxide | A | Calc'd 380.01, found 379.8 |
| 11 | 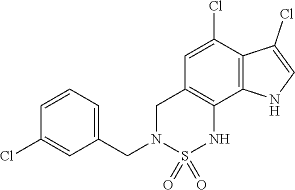 [SMILES: O=S1(=O)Nc2c(cc(Cl)c3c(Cl)c[nH]c23)CN1Cc1cccc(Cl)c1] | 6,7-dichloro-3-(3- chlorobenzyl)-1,3,4,9- tetrahydro-[1,2,6] thiadiazino[4,3-g]indole 2,2-dioxide | A | Calc'd 415.97, found 416.0 |
| 13 | 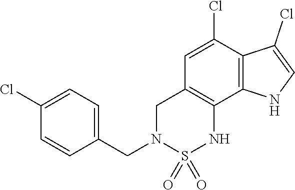 [SMILES: O=S1(=O)Nc2c(cc(Cl)c3c(Cl)c[nH]c23)CN1Cc1ccc(Cl)cc1] | 6,7-dichloro-3-(4- chlorobenzyl)-1,3,4,9- tetrahydro-[1,2,6] thiadiazino[4,3-g]indole 2,2-dioxide | A | Calc'd 415.97, found 416.0 |
| 14 | 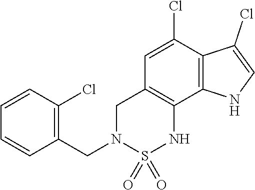 [SMILES: O=S1(=O)Nc2c(cc(Cl)c3c(Cl)c[nH]c23)CN1Cc1ccccc1Cl] | 6,7-dichloro-3-(2- chlorobenzyl)-1,3,4,9- tetrahydro-[1,2,6] thiadiazino[4,3-g] indole 2,2-dioxide | A | Calc'd 415.97, found 416.0 |
| 15 | 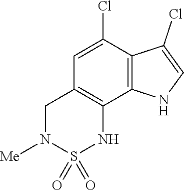 [SMILES: CN1Cc2cc(Cl)c3c(Cl)c[nH]c3c2NS1(=O)=O] | 6,7-dichloro-3-methyl- 1,3,4,9-tetrahydro- [1,2,6]thiadiazino [4,3-g]indole 2,2-dioxide | A | Calc'd 305.98, found 306.0 |
| 19 | 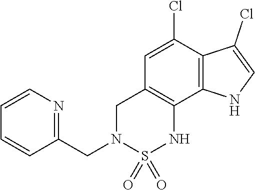 [SMILES: O=S1(=O)Nc2c(cc(Cl)c3c(Cl)c[nH]c23)CN1Cc1ccccn1] | 6,7-dichloro-3- (pyridin-2-ylmethyl)- 1,3,4,9-tetrahydro- [1,2,6]thiadiazino [4,3-g]indole 2,2-dioxide | A | Calc'd 383.0, found 383.0 |
| 20 | 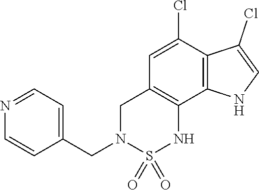 [SMILES: O=S1(=O)Nc2c(cc(Cl)c3c(Cl)c[nH]c23)CN1Cc1ccncc1] | 6,7-dichloro-3- (pyridin-4-ylmethyl)- 1,3,4,9- tetrahydro-[1,2,6] thiadiazino[4,3-g] indole 2,2-dioxide | A | Calc'd 383.0, found 383.0 |
| 21 | 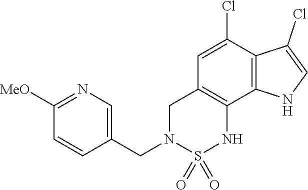 [SMILES: COc1ccc(CN2Cc3cc(Cl)c4c(Cl)c[nH]c4c3NS2(=O)=O)cn1] | 6,7-dichloro-3-((6- methoxypyridin-3-yl) methyl)-1,3,4,9-tetrahydro- [1,2,6]thiadiazino [4,3-g]indole 2,2-dioxide | A | Calc'd 413.02, found 413.0 |
| 23 | 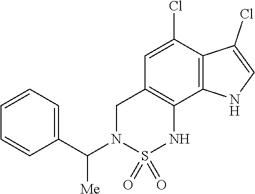 [SMILES: CC(c1ccccc1)N1Cc2cc(Cl)c3c(Cl)c[nH]c3c2NS1(=O)=O] | 6,7-dichloro-3-(1- phenylethyl)-1,3,4,9- tetrahydro-[1,2,6] thiadiazino[4,3-g] indole 2,2- dioxide | A | Calc'd 396.03, found 396.0 |
| 26 | 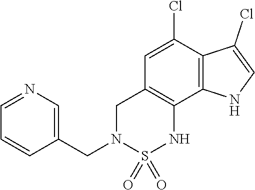 [SMILES: O=S1(=O)Nc2c(cc(Cl)c3c(Cl)c[nH]c23)CN1Cc1cccnc1] | 6,7-dichloro-3- (pyridin-3-ylmethyl)- 1,3,4,9- tetrahydro-[1,2,6] thiadiazino[4,3-g] indole 2,2-dioxide | A | Calc'd 383.0, found 383.0 |
| 27 | 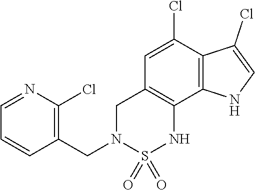 [SMILES: O=S1(=O)Nc2c(cc(Cl)c3c(Cl)c[nH]c23)CN1Cc1cccnc1Cl] | 6,7-dichloro-3-((2- chloropyridin-3-yl) methyl)-1,3,4,9-tetrahydro- [1,2,6]thiadiazino [4,3-g]indole 2,2-dioxide | A | Calc'd 416.97, found 417.0 |
| 33 | 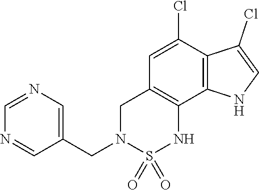 [SMILES: O=S1(=O)Nc2c(cc(Cl)c3c(Cl)c[nH]c23)CN1Cc1cncnc1] | 6,7-dichloro-3- (pyrimidin-5-ylmethyl)- 1,3,4,9-tetrahydro- [1,2,6]thiadiazino[4,3-g] indole 2,2-dioxide | A | Calc'd 384.0, found 384.0 |
| 35 | 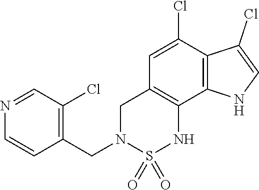 [SMILES: O=S1(=O)Nc2c(cc(Cl)c3c(Cl)c[nH]c23)CN1Cc1ccncc1Cl] | 6,7-dichloro-3-((3- chloropyridin-4-yl) methyl)-1,3,4,9-tetrahydro- [1,2,6]thiadiazino [4,3-g]indole 2,2-dioxide | A | Calc'd 416.9, found 417 |
| 36 | 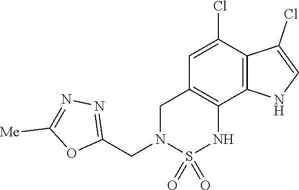 [SMILES: Cc1nnc(CN2Cc3cc(Cl)c4c(Cl)c[nH]c4c3NS2(=O)=O)o1] | 6,7-dichloro-3-((5- methyl-1,3,4-oxadiazol-2- yl)methyl)-1,3,4,9- tetrahydro-[1,2,6] thiadiazino[4,3-g]indole 2,2-dioxide | B | Calc'd 388.0, found 388.0 |
| 37 | 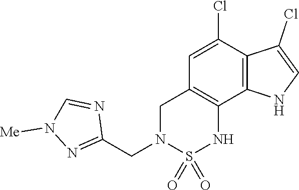 [SMILES: Cn1cnc(CN2Cc3cc(Cl)c4c(Cl)c[nH]c4c3NS2(=O)=O)n1] | 6,7-dichloro-3-((1- methyl-1H-1,2,4-triazol-3- yl)methyl)-1,3,4,9- tetrahydro-[1,2,6] thiadiazino[4,3-g]indole 2,2-dioxide | A | Calc'd 387.0, found 387.0 |
| 38 | 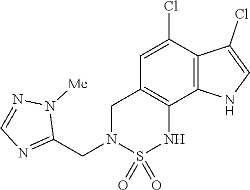 [SMILES: Cn1ncnc1CN1Cc2cc(Cl)c3c(Cl)c[nH]c3c2NS1(=O)=O] | 6,7-dichloro-3-((1- methyl-1H-1,2,4-triazol-5- yl)methyl)-1,3,4,9- tetrahydro-[1,2,6] thiadiazino[4,3-g]indole 2,2-dioxide | B | Calc'd 387.0, found 387.0 |
| 43 | 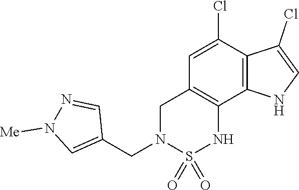 [SMILES: Cn1cc(CN2Cc3cc(Cl)c4c(Cl)c[nH]c4c3NS2(=O)=O)cn1] | 6,7-dichloro-3-((1- methyl-1H-pyrazol- 4-yl)methyl)-1,3,4,9- tetrahydro-[1,2,6] thiadiazino[4,3-g]indole 2,2-dioxide | A | Calc'd 386., found 386.0 |
| 46 | 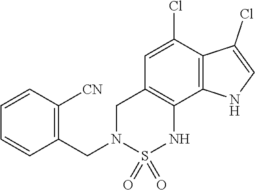 [SMILES: N#Cc1ccccc1CN1Cc2cc(Cl)c3c(Cl)c[nH]c3c2NS1(=O)=O] | 2-((6,7-dichloro-2,2- dioxido-4,9-dihydro- [1,2,6]thiadiazino[4,3-g] indol-3(1H)-yl)methyl) benzonitrile | A | Calc'd 407.01, found 407.0 |
| 48 | 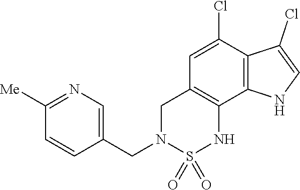 [SMILES: Cc1ccc(CN2Cc3cc(Cl)c4c(Cl)c[nH]c4c3NS2(=O)=O)cn1] | 6,7-dichloro-3-((6- methylpyridin-3-yl) methyl)-1,3,4,9- tetrahydro-[1,2,6] thiadiazino[4,3-g]indole 2,2-dioxide | A | Calc'd 397.02, found 397.0 |
| 49 | 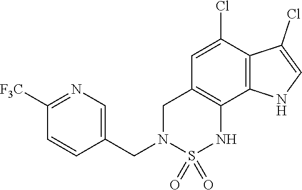 [SMILES: O=S1(=O)Nc2c(cc(Cl)c3c(Cl)c[nH]c23)CN1Cc1ccc(C(F)(F)F)nc1] | 6,7-dichloro-3-((6- (trifluoromethyl)pyridin- 3-yl)methyl)-1,3,4,9- tetrahydro-[1,2,6] thiadiazino[4,3-g]indole 2,2-dioxide | A | Calc'd 451.0, found 451.0 |
| 50 | 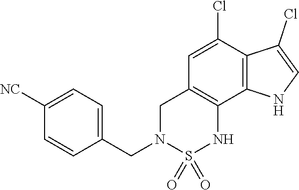 [SMILES: N#Cc1ccc(CN2Cc3cc(Cl)c4c(Cl)c[nH]c4c3NS2(=O)=O)cc1] | 4-((6,7-dichloro-2,2- dioxido-4,9-dihydro- [1,2,6]thiadiazino[4,3-g] indol-3(1H)-yl)methyl) benzonitrile | A | Calc'd 407.01, found 407.0 |
| 51 | 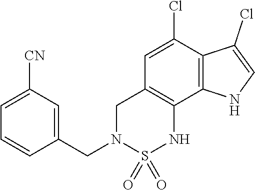 [SMILES: N#Cc1cccc(CN2Cc3cc(Cl)c4c(Cl)c[nH]c4c3NS2(=O)=O)c1] | 3-((6,7-dichloro-2,2- dioxido-4,9-dihydro- [1,2,6]thiadiazino[4,3-g] indol-3(1H)-yl)methyl) benzonitrile | A | Calcd'd 407.01, found 407.0 |
| 52 | 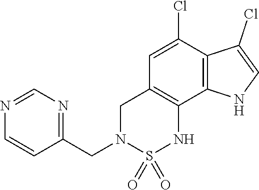 [SMILES: O=S1(=O)Nc2c(cc(Cl)c3c(Cl)c[nH]c23)CN1Cc1ccncn1] | 6,7-dichloro-3- (pyrimidin-4-ylmethyl)- 1,3,4,9-tetrahydro-[1,2,6] thiadiazino[4,3-g]indole 2,2-dioxide | B | Calc'd 384.0, found 384.0 |
| 53 | 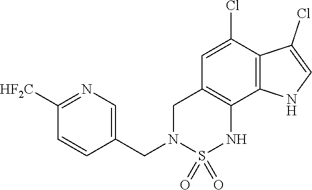 | 6,7-dichloro-3-((6- (difluoromethyl)pyridin-3- yl)methyl)-1,3,4,9- tetrahydro-[1,2,6]thiadiazino [4,3-g]indole 2,2-dioxide | A | Calc'd 433.00, found 433.0 |
| 54 | 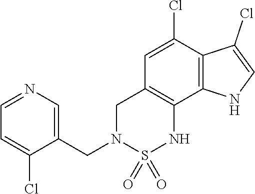 [SMILES: O=S1(=O)Nc2c(cc(Cl)c3c(Cl)c[nH]c23)CN1Cc1cnccc1Cl] | 6,7-dichloro-3-((4- chloropyridin-3-yl) methyl)-1,3,4,9- tetrahydro-[1,2,6] thiadiazino[4,3-g]indole 2,2-dioxide | A | Calc'd 416.97, found 417.0 |
| 55 | 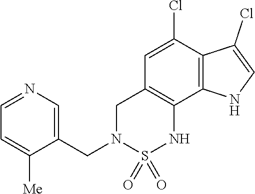 [SMILES: Cc1ccncc1CN1Cc2cc(Cl)c3c(Cl)c[nH]c3c2NS1(=O)=O] | 6,7-dichloro-3-((4- methylpyridin-3-yl) methyl)-1,3,4,9- tetrahydro-[1,2,6] thiadiazino[4,3-g]indole 2,2-dioxide | A | Calc'd 397.0, found 397.0 |
| 56 | 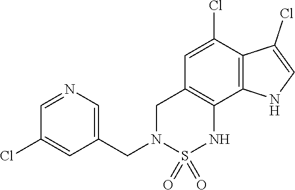 [SMILES: O=S1(=O)Nc2c(cc(Cl)c3c(Cl)c[nH]c23)CN1Cc1cncc(Cl)c1] | 6,7-dichloro-3-((5- chloropyridin-3-yl)methyl)- 1,3,4,9- tetrahydro-[1,2,6] thiadiazino[4,3-g]indole 2,2-dioxide | A | Calc'd 416.97, found 417.0 |
| 59 | 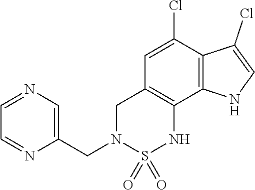 [SMILES: O=S1(=O)Nc2c(cc(Cl)c3c(Cl)c[nH]c23)CN1Cc1cnccn1] | 6,7-dichloro-3-(pyrazin- 2-ylmethyl)- 1,3,4,9- tetrahydro-[1,2,6] thiadiazino[4,3-g]indole 2,2-dioxide | B | Calc'd 384.0, found 384.0 |
| 63 | 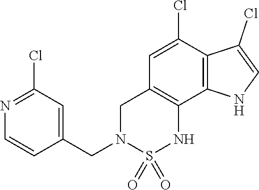 [SMILES: O=S1(=O)Nc2c(cc(Cl)c3c(Cl)c[nH]c23)CN1Cc1ccnc(Cl)c1] | 6,7-dichloro-3-((2- chloropyridin-4-yl)methyl)- 1,3,4,9- tetrahydro-[1,2,6] thiadiazino[4,3-g]indole 2,2-dioxide | A | Calc'd 416.97, found 417.0 |
| 67 | 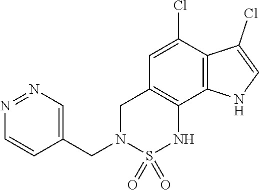 [SMILES: O=S1(=O)Nc2c(cc(Cl)c3c(Cl)c[nH]c23)CN1Cc1ccnnc1] | 6,7-dichloro-3-(pyridazin- 4-ylmethyl)- 1,3,4,9- tetrahydro-[1,2,6] thiadiazino[4,3-g]indole 2,2-dioxide | B | Calc'd 384.0, found 384.0 |
| 69 | 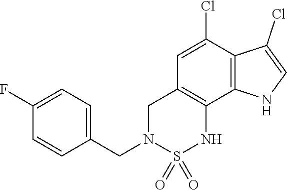 [SMILES: O=S1(=O)Nc2c(cc(Cl)c3c(Cl)c[nH]c23)CN1Cc1ccc(F)cc1] | 6,7-dichloro-3-(4- fluorobenzyl)- 1,3,4,9- tetrahydro-[1,2,6] thiadiazino[4,3-g]indole 2,2-dioxide | A | Calc'd 400, found 400.0 |
| 70 | 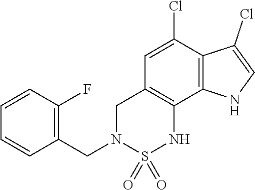 [SMILES: O=S1(=O)Nc2c(cc(Cl)c3c(Cl)c[nH]c23)CN1Cc1ccccc1F] | 6,7-dichloro-3-(2- fluorobenzyl)- 1,3,4,9- tetrahydro-[1,2,6] thiadiazino[4,3-g]indole 2,2-dioxide | A | Calc'd 400.0, found 400.0 |
| 73 | 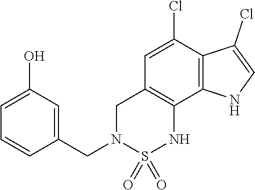 [SMILES: O=S1(=O)Nc2c(cc(Cl)c3c(Cl)c[nH]c23)CN1Cc1cccc(O)c1] | 6,7-dichloro-3-(3- hydroxybenzyl)- 1,3,4,9- tetrahydro-[1,2,6] thiadiazino[4,3-g]indole 2,2-dioxide | B | Calc'd 398.01, found 398.0 |
| 75 | 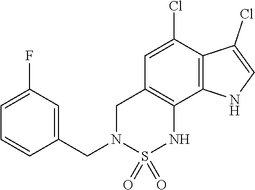 [SMILES: O=S1(=O)Nc2c(cc(Cl)c3c(Cl)c[nH]c23)CN1Cc1cccc(F)c1] | 6,7-dichloro-3-(3- fluorobenzyl)- 1,3,4,9- tetrahydro-[1,2,6] thiadiazino[4,3-g]indole 2,2-dioxide | A | Calc'd 400.0, found 400.0 |
| 92 | 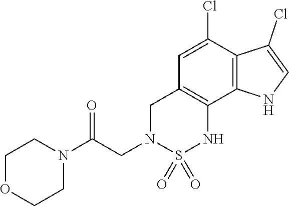 [SMILES: O=C(CN1Cc2cc(Cl)c3c(Cl)c[nH]c3c2NS1(=O)=O)N1CCOCC1] | 2-(6,7-dichloro-2,2- dioxido-4,9-dihydro- [1,2,6]thiadiazino[4,3-g] indol-3(1H)-yl)-1- morpholinoethan-1- one | A | Calc'd 419.0, found 419.0 |
| 93 | 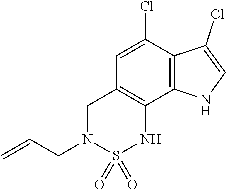 [SMILES: C=CCN1Cc2cc(Cl)c3c(Cl)c[nH]c3c2NS1(=O)=O] | 3-allyl-6,7-dichloro- 1,3,4,9- tetrahydro-[1,2,6] thiadiazino[4,3-g]indole 2,2-dioxide | A | Calc'd 329.99, found 329.8 |

### Example 3

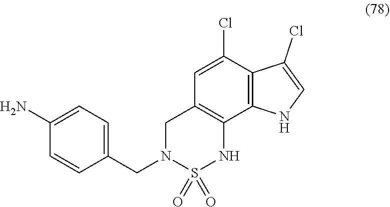 [SMILES: Nc1ccc(CN2Cc3cc(Cl)c4c(Cl)c[nH]c4c3NS2(=O)=O)cc1]

### 3-(4-aminobenzyl)-6,7-dichloro-1,3,4,9-tetrahydro-[1,2,6]thiadiazino[4,3-g]indole 2,2-dioxide (Compound 78)

Compound 78 was prepared in an analogous manner to that outlined in Scheme 2.

### Step 1. 1-allyl-3-(4-aminobenzyl)-6,7-dichloro-1,3,4,9-tetrahydro-[1,2,6]thiadiazino[4,3-g]indole 2,2-dioxide

The starting material 1-allyl-6,7-dichloro-3-(4-nitrobenzyl)-1,3,4,9-tetrahydro-[1,2,6]thiadiazino[4,3-g]indole 2,2-dioxide was prepared using the procedure outlined for Compound 12 and 1-(bromomethyl)-4-nitrobenzene. To a solution of 1-allyl-6,7-dichloro-3-(4-nitrobenzyl)-1,3,4,9-tetrahydro-[1,2,6]thiadiazino[4,3-g]indole 2,2-dioxide (24.0 mg, 0.051 mmol) in ethanol (4.0 mL) and water (0.8 mL) was added Fe powder (14.4 mg, 0.26 mmol) and NH₄Cl (27.73 mg, 0.51 mmol). The mixture was stirred at 80° C. for 3 hrs, the mixture was filtered through celite, the mixture was concentrated in vacuum, and the residue was partitioned between EtOAc and H₂O. The organic layer was dried over Na₂SO₄, filtered, concentrated and purified by Prep-TLC (silica gel, EtOAc/PE=1/2) to give 1-allyl-3-(4-aminobenzyl)-6,7-dichloro-1,3,4,9-tetrahydro-[1,2,6]thiadiazino[4,3-g]indole 2,2-dioxide. MS=437.0 (M+1).

### Step 2. 3-(4-aminobenzyl)-6,7-dichloro-1,3,4,9-tetrahydro-[1,2,6]thiadiazino[4,3-g]indole 2,2-dioxide (78)

Utilizing analogous processes to those described for Compounds 12 and 42, Deprotection Method A was used. The product was purified by Prep-HPLC to afford 3-(4-aminobenzyl)-6,7-dichloro-1,3,4,9-tetrahydro-[1,2,6]thiadiazino[4,3-g]indole 2,2-dioxide (78). ¹H NMR (400 MHz, DMSO-d6): δ 7.33 (s, 1H), 7.00 (m, 2H), 6.70-6.75 (m, 3H), 4.47 (s, 2H), 3.95 (s, 2H). MS=397.1 (M+1).

TABLE 2
The following compounds were prepared using procedures similar to those
described for Example 3 (Compound 78) using the appropriate starting materials.

| Compd. | | | Exact Mass |
|---|---|---|---|
| No. | Structure | Name | [M + H]⁺ |
| 74 | 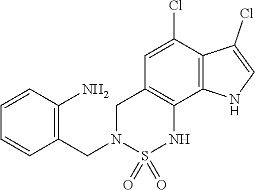 [SMILES: Nc1ccccc1CN1Cc2cc(Cl)c3c(Cl)c[nH]c3c2NS1(=O)=O] | 3-(2-aminobenzyl)-6,7-dichloro- 1,3,4,9-tetrahydro-[1,2,6] thiadiazino[4,3-g]indole 2,2- dioxide | Calc'd 397.02, found 397.1 |
| 76 | 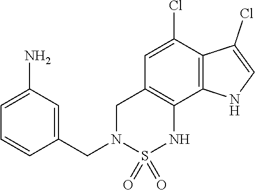 [SMILES: Nc1cccc(CN2Cc3cc(Cl)c4c(Cl)c[nH]c4c3NS2(=O)=O)c1] | 3-(3-aminobenzyl)-6,7-dichloro- 1,3,4,9-tetrahydro-[1,2,6] thiadiazino[4,3-g]indole 2,2- dioxide | Calc'd 397.02, found 397.1 |

### Example 4

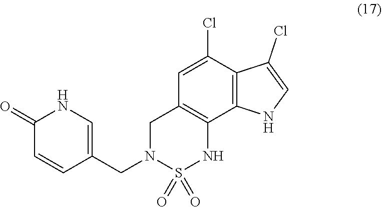 [SMILES: O=c1ccc(CN2Cc3cc(Cl)c4c(Cl)c[nH]c4c3NS2(=O)=O)c[nH]1]

### 5-((6,7-dichloro-2,2-dioxido-4,9-dihydro-[1,2,6]thiadiazino[4,3-g]indol-3(1H)-yl)methyl)pyridin-2(1H)-one (Compound 17)

Compound 17 was prepared in an analogous manner to that outlined in Scheme 3.

### Step 1. 5-((1-allyl-6-chloro-2,2-dioxido-4,9-dihydro-[1,2,6]thiadiazino[4,3-g]indol-3(1H)-yl)methyl)pyridin-2(1H)-one

The starting material 1-allyl-6,7-dichloro-3-((6-methoxypyridin-3-yl)methyl)-1,3,4,9-tetrahydro-[1,2,6]thiadiazino[4,3-g]indole 2,2-dioxide was prepared using the procedure outlined in Example 1. To a solution of 1-allyl-6,7-dichloro-3-((6-methoxypyridin-3-yl)methyl)-1,3,4,9-tetrahydro-[1,2,6]thiadiazino[4,3-g]indole 2,2-dioxide (132.0 mg, 0.29 mmol) in MeCN (10 mL) was added NaI (2.5 eq) and TMSCl (2.5 eq.) at RT. The mixture was stirred at 40° C. overnight. Then water (30 mL) was added to quench the reaction, and the product was extracted with EtOAc (3×15 mL), dried over anhydrous Na₂SO₄, filtered and the filtrate was concentrated and purified by HPLC (EtOAc/PE=2/1) to afford 5-((1-allyl-6-chloro-2,2-dioxido-4,9-dihydro-[1,2,6]thiadiazino[4,3-g]indol-3(1H)-yl)methyl)pyridin-2(1H)-one. MS=405.0 (M+1).

### Step 2. 5-((1-allyl-6,7-dichloro-2,2-dioxido-4,9-dihydro-[1,2,6]thiadiazino[4,3-g]indol-3(1H)-yl)methyl)pyridin-2(1H)-one

5-((1-allyl-6-chloro-2,2-dioxido-4,9-dihydro-[1,2,6]thiadiazino[4,3-g]indol-3(1H)-yl)methyl)pyridin-2(1H)-one (22.0 mg, 0.060 mmol) was dissolved in THF (5 mL)/DMF (0.1 mL), then NCS (10.7 mg, 0.08 mmol) was added at −10° C. and the solution was stirred at 25° C. for 4 h. After the reaction was complete, Na₂SO₃ (2 mL, saturated aqueous) was added and the resulting mixture stirred for 10 min. The mixture was concentrated and diluted with water. The resulting mixture was extracted with 3×30 mL EtOAc. The combined organics were rinsed with 30 mL of brine, dried over Na₂SO₄, the filtrate was concentrated and purified by Prep-TLC (EtOAc/PE=1/2) to afford 5-((1-allyl-6,7-dichloro-2,2-dioxido-4,9-dihydro-[1,2,6]thiadiazino[4,3-g]indol-3(1H)-yl)methyl)pyridin-2(1H)-one.

### Step 3. 5-((6,7-dichloro-2,2-dioxido-4,9-dihydro-[1,2,6]thiadiazino[4,3-g]indol-3(1H)-yl)methyl)pyridin-2(1H)-one (17)

Utilizing an analogous method to that outlined in Example 2, for compound 42, Deprotection Method B, The reaction product was purified by Prep-HPLC to afford 5-((6,7-dichloro-2,2-dioxido-4,9-dihydro-[1,2,6]thiadiazino[4,3-g]indol-3(1H)-yl)methyl)pyridin-2(1H)-one (17). ¹H NMR (400 MHz, CD3OD): δ 7.64 (dd, J=9.2, 2.4 Hz, 1H), 7.33 (s, 2H), 6.86 (s, 1H), 6.56 (d, J=9.6 Hz, 1H), 4.61 (s, 2H), 3.95 (s, 2H). MS=399.0 (M+1).

TABLE 3
The following compounds were prepared using procedures similar to those
described for Example 4, compound 17 using the appropriate starting materials.

| Compd. | | | Exact Mass |
|---|---|---|---|
| No. | Structure | Name | [M + H]⁺ |
| 57 | 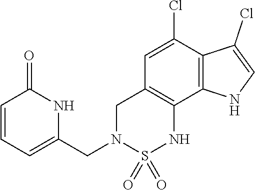 [SMILES: O=c1cccc(CN2Cc3cc(Cl)c4c(Cl)c[nH]c4c3NS2(=O)=O)[nH]1] | 6-((6,7-dichloro-2,2-dioxido-4,9- dihydro-[1,2,6]thiadiazino[4,3-g]indol- 3(1H)-yl)methyl)pyridin-2(1H)-one | Calc'd 399.00, found 399.0 |
| 60 | 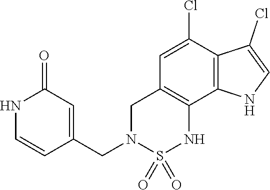 [SMILES: O=c1cc(CN2Cc3cc(Cl)c4c(Cl)c[nH]c4c3NS2(=O)=O)cc[nH]1] | 4-((6,7-dichloro-2,2-dioxido-4,9- dihydro-[1,2,6]thiadiazino[4,3-g]indol- 3(1H)-yl)methyl)pyridin-2(1H)-one | Calc'd 399.00, found 399.0 |
| 97 | 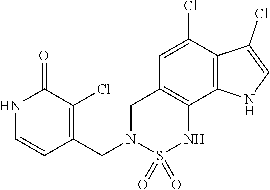 [SMILES: O=c1[nH]ccc(CN2Cc3cc(Cl)c4c(Cl)c[nH]c4c3NS2(=O)=O)c1Cl] | 3-chloro-4-((6,7-dichloro-2,2-dioxido- 4,9-dihydro-[1,2,6]thiadiazino [4,3-g]indol-3(1H)-yl)methyl)pyridin-2(1H)- one | Calc'd 432.96, found 433.0 |
| 98 | 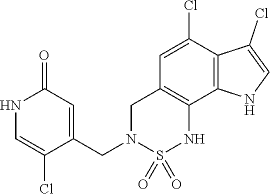 [SMILES: O=c1cc(CN2Cc3cc(Cl)c4c(Cl)c[nH]c4c3NS2(=O)=O)c(Cl)c[nH]1] | 5-chloro-4-((6,7-dichloro-2,2-dioxido- 4,9-dihydro-[1,2,6]thiadiazino [4,3-g]indol-3(1H)-yl)methyl)pyridin-2(1H)- one | Calc'd 432.96, found 433.0 |

### Example 5

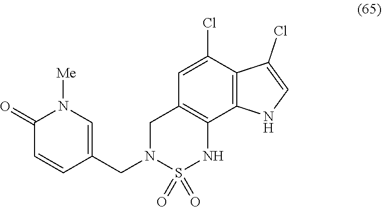 [SMILES: Cn1cc(CN2Cc3cc(Cl)c4c(Cl)c[nH]c4c3NS2(=O)=O)ccc1=O]

### 5-((6,7-dichloro-2,2-dioxido-4,9-dihydro-[1,2,6]thiadiazino[4,3-g]indol-3(1H)-yl)methyl)-1-methylpyridin-2(1H)-one (Compound 65)

Compound 65 was prepared in an analogous manner to that outlined in Scheme 4.

### Step 1. 5-((1-allyl-6-chloro-2,2-dioxido-4,9-dihydro-[1,2,6]thiadiazino[4,3-g]indol-3(1H)-yl)methyl)-1-methylpyridin-2(1H)-one

The starting material 5-((1-allyl-6-chloro-2,2-dioxido-4,9-dihydro-[1,2,6]thiadiazino[4,3-g]indol-3(1H)-yl)methyl)pyridin-2(1H)-one was prepared using the procedure outlined in Example 17. To a solution of 5-((1-allyl-6-chloro-2,2-dioxido-4,9-dihydro-[1,2,6]thiadiazino[4,3-g]indol-3(1H)-yl)methyl)pyridin-2(1H)-one (59 mg, 0.15 mmol) in DMF (6 mL) was added K₂CO₃ (1.2 eq.) at 0° C. for 0.5 h. Then iodomethane (10 μL, 0.16 mmol) was added and stirred at RT for 2 h. The mixture was diluted with water (60 mL) and extracted with EtOAc (3×10 mL), dried over anhydrous Na₂SO₄, filtered, the filtrate was concentrated and purified by Prep-TLC (EtOAc/PE=1/1) to afford slightly impure 5-((1-allyl-6-chloro-2,2-dioxido-4,9-dihydro-[1,2,6]thiadiazino[4,3-g]indol-3(1H)-yl)methyl)-1-methylpyridin-2(1H)-one. MS=417.1 (M−1).

### Step 2. 5-((1-allyl-6,7-dichloro-2,2-dioxido-4,9-dihydro-[1,2,6]thiadiazino[4,3-g]indol-3(1H)-yl)methyl)pyridin-2(1H)-one

Synthesis of 5-((1-allyl-6,7-dichloro-2,2-dioxido-4,9-dihydro-[1,2,6]thiadiazino[4,3-g]indol-3(1H)-yl)methyl)pyridin-2(1H)-one from 5-((1-allyl-6-chloro-2,2-dioxido-4,9-dihydro-[1,2,6]thiadiazino[4,3-g]indol-3(1H)-yl)methyl)-1-methylpyridin-2(1H)-one was achieved in a similar manner to step 2 from Example 4, compound 17. MS=451.0 (M−1).

### Step 3. 5-((6,7-dichloro-2,2-dioxido-4,9-dihydro-[1,2,6]thiadiazino[4,3-g]indol-3(1H)-yl)methyl)-1-methylpyridin-2(1H)-one (65)

Synthesis of 5-((6,7-dichloro-2,2-dioxido-4,9-dihydro-[1,2,6]thiadiazino[4,3-g]indol-3(1H)-yl)methyl)-1-methylpyridin-2(1H)-one from 5-((1-allyl-6,7-dichloro-2,2-dioxido-4,9-dihydro-[1,2,6]thiadiazino[4,3-g]indol-3(1H)-yl)methyl)pyridin-2(1H)-one (65) was achieved in a similar manner to step 3 from Example 4, compound 17. ¹H NMR (400 MHz, CD₃OD): δ 7.59 (d, J=7.6 Hz, 1H), 7.58 (s, 1H), 7.34 (s, 1H), 6.87 (s, 1H), 6.57 (d, J=9.0 Hz, 1H), 4.63 (s, 2H), 3.96 (s, 2H), 3.54 (s, 3H). MS=413.0 (M+1).

TABLE 4
The following compounds were prepared using procedures similar to those
described for Example 5, Compound 65, using the appropriate starting materials.

| Compd. | | | Exact Mass |
|---|---|---|---|
| No. | Structure | Name | [M + H]⁺ |
| 61 | 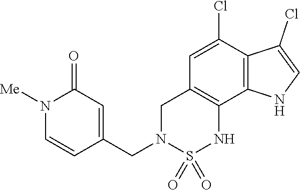 [SMILES: Cn1ccc(CN2Cc3cc(Cl)c4c(Cl)c[nH]c4c3NS2(=O)=O)cc1=O] | 4-((6,7-dichloro-2,2-dioxido-4,9- dihydro-[1,2,6]thiadiazino[4,3-g] indol-3(1H)-yl)methyl)-1-methylpyridin- 2(1H)-one | Calc'd 413.02, found 413.0 |
| 62 | 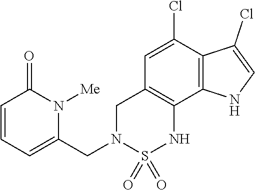 [SMILES: Cn1c(CN2Cc3cc(Cl)c4c(Cl)c[nH]c4c3NS2(=O)=O)cccc1=O] | 6-((6,7-dichloro-2,2-dioxido-4,9- dihydro-[1,2,6]thiadiazino[4,3-g] indol-3(1H)-yl)methyl)-1-methylpyridin- 2(1H)-one | Calc'd 413.02, found 413.0 |

### Examples 6 and 7

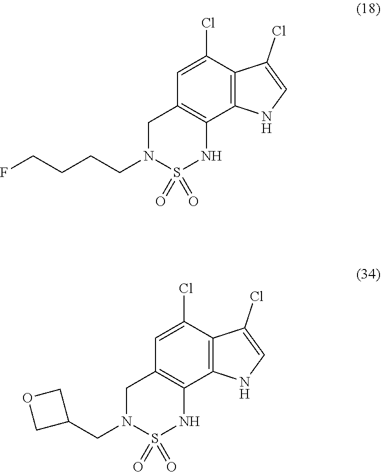 [SMILES: O=S1(=O)Nc2c(cc(Cl)c3c(Cl)c[nH]c23)CN1CCCCF ; O=S1(=O)Nc2c(cc(Cl)c3c(Cl)c[nH]c23)CN1CC1COC1]

### 6,7-dichloro-3-(4-fluorobutyl)-1,3,4,9-tetrahydro-[1,2,6]thiadiazino[4,3-g]indole 2,2-dioxide

(Compound 18) and 6,7-dichloro-3-(oxetan-3-ylmethyl)-1,3,4,9-tetrahydro-[1,2,6]thiadiazino[4,3-g]indole 2,2-dioxide (Compound 34) Compounds 18 and 34 were prepared in an analogous manner to that outlined in Scheme 5. For Compound 18:

### Step 1. 1-allyl-6-chloro-3-(4-fluorobutyl)-8-nitro-3,4-dihydro-1H-benzo[c][1,2,6]thiadiazine 2,2-dioxide (18-A)

Mesylate/Halide alkylation: To a solution of 1-allyl-6-chloro-8-nitro-3,4-dihydro-1H-benzo[c][1,2,6]thiadiazine 2,2-dioxide (200 mg, 0.66 mmol) in DMF (25 mL) was added NaH (2.0 equiv). The mixture was stirred at rt for 0.5 h. Then 4-fluorobutyl methanesulfonate (450 mg, 2.64 mmol) was added and stirred at 50° C. overnight, water (200 mL) was added, and the product was extracted with EtOAc (3×15 mL), dried over anhydrous Na₂SO₄, filtered, the filtrate was concentrated and purified by Prep-TLC (EtOAc/PE=1/3) to afford 1-allyl-6-chloro-3-(4-fluorobutyl)-8-nitro-3,4-dihydro-1H-benzo[c][1,2,6]thiadiazine 2,2-dioxide (18-A). MS=378.0 (M+1).

### Step 2. 1-allyl-6-chloro-3-(4-fluorobutyl)-1,3,4,9-tetrahydro-[1,2,6]thiadiazino[4,3-g]indole 2,2-dioxide (18-B)

1-allyl-6-chloro-3-(4-fluorobutyl)-8-nitro-3,4-dihydro-1H-benzo[c][1,2,6]thiadiazine 2,2-dioxide (18-A) was converted to 1-allyl-6-chloro-3-(4-fluorobutyl)-1,3,4,9-tetrahydro-[1,2,6]thiadiazino[4,3-g]indole 2,2-dioxide following the procedure outlined in step 5 of Preparatory Example 2. Purification by Prep-TLC (EtOAc/PE=1/2) gave 1-allyl-6-chloro-3-(4-fluorobutyl)-1,3,4,9-tetrahydro-[1,2,6]thiadiazino[4,3-g]indole 2,2-dioxide (18-B). MS=372.1 (M+1).

### Step 3. 1-allyl-6,7-dichloro-3-(4-fluorobutyl)-1,3,4,9-tetrahydro-[1,2,6]thiadiazino[4,3-g]indole 2,2-dioxide (18-C)

1-allyl-6-chloro-3-(4-fluorobutyl)-1,3,4,9-tetrahydro-[1,2,6]thiadiazino[4,3-g]indole 2,2-dioxide was converted to 1-allyl-6,7-dichloro-3-(4-fluorobutyl)-1,3,4,9-tetrahydro-[1,2,6]thiadiazino[4,3-g]indole 2,2-dioxide following the procedure outlined in step 6 of Preparatory Example 3. Purification by Prep-TLC (EtOAc/PE=1/2) gave 1-allyl-6,7-dichloro-3-(4-fluorobutyl)-1,3,4,9-tetrahydro-[1,2,6]thiadiazino[4,3-g]indole 2,2-dioxide (18-C). MS=406.0 (M+1).

### Step 4. 6,7-dichloro-3-(4-fluorobutyl)-1,3,4,9-tetrahydro-[1,2,6]thiadiazino[4,3-g]indole 2,2-dioxide (18)

Deprotection Method A from Examples 1 and 2 (compounds 12 and 42) was used. Purification by Prep-HPLC afforded 6,7-dichloro-3-(4-fluorobutyl)-1,3,4,9-tetrahydro-[1,2,6]thiadiazino[4,3-g]indole 2,2-dioxide. by Prep-HPLC to afford 6,7-dichloro-3-(4-fluorobutyl)-4,9-dihydro-1H-pyrrolo[3,2-h][2,1,3]benzothiadiazine 2,2-dioxide (18). ¹H NMR (400 MHz, CD₃OD) δ 7.31 (s, 1H), 6.88 (s, 1H), 4.71 (s, 2H), 4.52 (t, J=5.6 Hz, 1H), 4.40 (t, J=5.6 Hz, 1H), 3.02 (t, J=6.4 Hz, 2H), 1.82-1.70 (m, 4H). MS=366.0 (M+1).

For compound 34:

### Step 1: 1-allyl-6-chloro-8-nitro-3-(oxetan-3-ylmethyl)-3,4-dihydro-1H-benzo[c][1,2,6]thiadiazine 2,2-dioxide (34-A)

Mitsunobu alkylation: A solution of 1-allyl-6-chloro-8-nitro-3,4-dihydro-1H-benzo[c][1,2,6]thiadiazine 2,2-dioxide (350 mg, 1.15 mmol), 3-Oxetanemethanol (101.54 mg, 1.15 mmol) and PPh₃ (6.0 eq) in THF (10 mL) at 0° C. was treated with DIAD (6.0 eq). The mixture was stirred at 25° C. for 1 h. The reaction mixture was quenched by the addition of water (30 mL). The resulting mixture was extracted with EtOAc (3×15 mL). The combined organics were rinsed with 30 mL of brine, dried over MgSO₄, filtered, concentrated and purified by Prep-TLC (EtOAc/PE=1/2) to give slightly impure 1-allyl-6-chloro-8-nitro-3-(oxetan-3-ylmethyl)-3,4-dihydro-1H-benzo[c][1,2,6]thiadiazine 2,2-dioxide (34-A). MS=408.0 (M+Cl⁻).

### Step 2. 1-allyl-6-chloro-3-(oxetan-3-ylmethyl)-1,3,4,9-tetrahydro-[1,2,6]thiadiazino[4,3-g]indole 2,2-dioxide (34-B)

1-allyl-6-chloro-8-nitro-3-(oxetan-3-ylmethyl)-3,4-dihydro-1H-benzo[c][1,2,6]thiadiazine 2,2-dioxide (34-A) was converted to 1-allyl-6-chloro-3-(oxetan-3-ylmethyl)-1,3,4,9-tetrahydro-[1,2,6]thiadiazino[4,3-g]indole 2,2-dioxide (34-B) following the procedure outlined in step 5 of Preparatory Example 2. MS=366.1 (M−1).

### Step 3. 1-allyl-6,7-dichloro-3-(oxetan-3-ylmethyl)-1,3,4,9-tetrahydro-[1,2,6]thiadiazino[4,3-g]indole 2,2-dioxide (34-C)

1-allyl-6-chloro-3-(oxetan-3-ylmethyl)-1,3,4,9-tetrahydro-[1,2,6]thiadiazino[4,3-g]indole 2,2-dioxide (34-B) was converted to 1-allyl-6,7-dichloro-3-(oxetan-3-ylmethyl)-1,3,4,9-tetrahydro-[1,2,6]thiadiazino[4,3-g]indole 2,2-dioxide (34-C) following the procedure outlined in step 6 of Preparatory Example 3. MS=400.0 (M−1).

### Step 4. 1-allyl-6,7-dichloro-3-(oxetan-3-ylmethyl)-1,3,4,9-tetrahydro-[1,2,6]thiadiazino[4,3-g]indole 2,2-dioxide (34)

Deprotection Method A from Examples 1 and 2, compounds 12 and 42 were used. Purification by Prep-HPLC afforded 1-allyl-6,7-dichloro-3-(oxetan-3-ylmethyl)-1,3,4,9-tetrahydro-[1,2,6]thiadiazino[4,3-g]indole 2,2-dioxide (34). H NMR (400 MHz, CD₃OD): δ 7.32 (s, 1H), 6.87 (s, 1H), 4.76-4.81 (m, 2H), 4.68 (s, 2H), 4.42 (t, J=6.0 Hz, 2H), 3.33-3.42 (m, 1H), 3.29-3.33 (n, 2H). MS=362.0 (M+1).

TABLE 5
The following compounds were prepared using procedures similar to those
described for Examples 6 and 7 (compounds 18 and 34) using the appropriate starting materials.

| | | | | Exact Mass |
|---|---|---|---|---|
| Compd. | | | Alkylation | [M + H]⁺ or |
| No. | Structure | Name | Method | [M − H]⁻ |
| 1 | 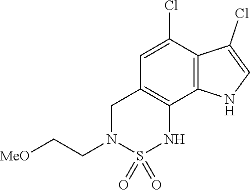 [SMILES: COCCN1Cc2cc(Cl)c3c(Cl)c[nH]c3c2NS1(=O)=O] | 6,7-dichloro-3-(2- methoxyethyl)- 1,3,4,9-tetrahydro-[1,2,6] thiadiazino[4,3-g]indole 2,2- dioxide | Halide (Br) | Calc'd 350.0, found 349.8 |
| 2 | 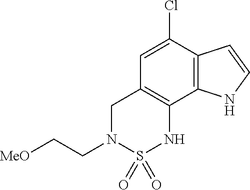 [SMILES: COCCN1Cc2cc(Cl)c3cc[nH]c3c2NS1(=O)=O] | 6-chloro-3-(2-methoxyethyl)-1,3,4,9- tetrahydro-[1,2,6] thiadiazino[4,3-g]indole 2,2- dioxide | Halide (Br, no NCS step) | Calc'd 316.0, found 315.9 |
| 25 | 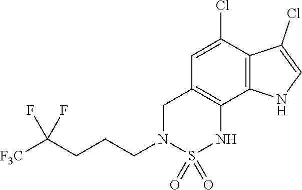 [SMILES: O=S1(=O)Nc2c(cc(Cl)c3c(Cl)c[nH]c23)CN1CCCC(F)(F)C(F)(F)F] | 6,7-dichloro-3-(4,4,5, 5,5-pentafluoropentyl)-1,3,4,9- tetrahydro-[1,2,6] thiadiazino[4,3-g]indole 2,2- dioxide | Mesylate | Calc'd 451.9, found 452.0 |
| 32 | 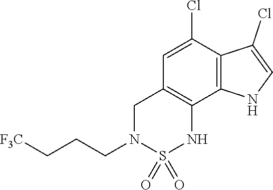 [SMILES: O=S1(=O)Nc2c(cc(Cl)c3c(Cl)c[nH]c23)CN1CCCC(F)(F)F] | 6,7-dichloro-3-(4,4,4- trifluorobutyl)-1,3,4,9- tetrahydro-[1,2,6] thiadiazino[4,3-g]indole 2,2- dioxide | Mesylate | Calc'd 402.0, found 402.0 |
| 40 | 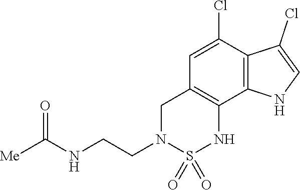 [SMILES: CC(=O)NCCN1Cc2cc(Cl)c3c(Cl)c[nH]c3c2NS1(=O)=O] | N-(2-(6,7-dichloro-2,2- dioxido-4,9-dihydro-[1,2,6] thiadiazino[4,3- g]indol-3(1H)-yl)ethyl) acetamide | Mitsunobu | Calc'd 377.0, found 377.0. |
| 41 | 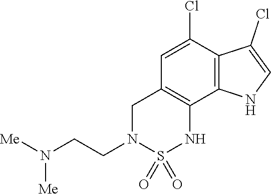 [SMILES: CN(C)CCN1Cc2cc(Cl)c3c(Cl)c[nH]c3c2NS1(=O)=O] | 6,7-dichloro-3-(2- (dimethylamino)ethyl)-1,3,4,9- tetrahydro-[1,2,6] thiadiazino[4,3-g]indole 2,2- dioxide | Mitsunobu | Calc'd 363.0, found 363.0 |
| 44 | 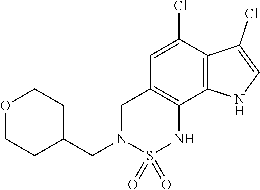 [SMILES: O=S1(=O)Nc2c(cc(Cl)c3c(Cl)c[nH]c23)CN1CC1CCOCC1] | 6,7-dichloro-3- ((tetrahydro-2H-pyran-4-yl) methyl)-1,3,4,9- tetrahydro-[1,2,6] thiadiazino[4,3-g]indole 2,2- dioxide | Mesylate | Calc'd 390.0, found 390.0 |
| 68 | 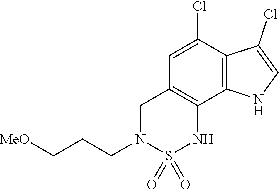 [SMILES: COCCCN1Cc2cc(Cl)c3c(Cl)c[nH]c3c2NS1(=O)=O] | 6,7-dichloro-3-(3- methoxypropyl)-1,3,4,9- tetrahydro-[1,2,6] thiadiazino[4,3-g]indole 2,2- dioxide | Mesylate | Calc'd 364.0, found 364.0 |
| 71 | 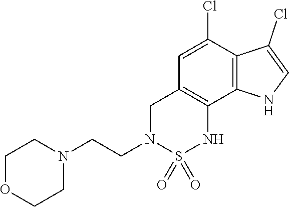 [SMILES: O=S1(=O)Nc2c(cc(Cl)c3c(Cl)c[nH]c23)CN1CCN1CCOCC1] | 6,7-dichloro-3-(2- morpholinoethyl)-1,3,4,9- tetrahydro-[1,2,6] thiadiazino[4,3-g]indole 2,2- dioxide | Mitsunobu | Calc'd 405.0, found 405.0 |
| 77 | 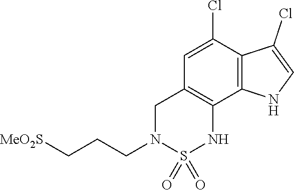 [SMILES: CS(=O)(=O)CCCN1Cc2cc(Cl)c3c(Cl)c[nH]c3c2NS1(=O)=O] | 6,7-dichloro-3-(3- (methylsulfonyl)propyl)-1,3,4,9- tetrahydro-[1,2,6] thiadiazino[4,3-g]indole 2,2- dioxide | Mitsunobu | Calc'd 411.9, found 412.0 |
| 79 | 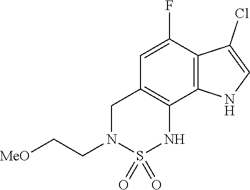 [SMILES: COCCN1Cc2cc(F)c3c(Cl)c[nH]c3c2NS1(=O)=O] | 7-chloro-6-fluoro-3-(2- methoxyethyl)-1,3,4,9- tetrahydro-[1,2,6] thiadiazino[4,3-g]indole 2,2- dioxide | Halide (Br) | Calc'd 332.0, found 332.0 |
| 81 | 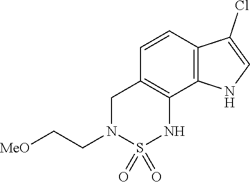 [SMILES: COCCN1Cc2ccc3c(Cl)c[nH]c3c2NS1(=O)=O] | 7-chloro-3-(2-methoxyethyl)- 1,3,4,9-tetrahydro-[1,2,6] thiadiazino[4,3-g]indole 2,2- dioxide | Halide (Br) | Calc'd 316.0, found 315.9. |
| 84 | 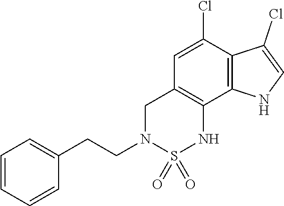 [SMILES: O=S1(=O)Nc2c(cc(Cl)c3c(Cl)c[nH]c23)CN1CCc1ccccc1] | 6,7-dichloro-3-phenethyl- 1,3,4,9-tetrahydro-[1,2,6] thiadiazino[4,3-g]indole 2,2- dioxide | Mesylate | Calc'd 396.0, found 396.0 |
| 85 | 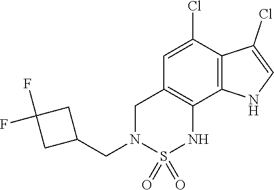 [SMILES: O=S1(=O)Nc2c(cc(Cl)c3c(Cl)c[nH]c23)CN1CC1CC(F)(F)C1] | 6,7-dichloro-3-((3,3- difluorocyclobutyl)methyl)- 1,3,4,9-tetrahydro-[1,2,6] thiadiazino[4,3-g]indole 2,2- dioxide | Mitsunobu | Calc'd 396.0, found 396.0 |
| 86 | 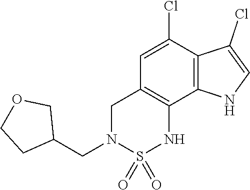 [SMILES: O=S1(=O)Nc2c(cc(Cl)c3c(Cl)c[nH]c23)CN1CC1CCOC1] | 6,7-dichloro-3- ((tetrahydrofuran-3-yl)methyl)- 1,3,4,9-tetrahydro-[1,2,6] thiadiazino[4,3-g]indole 2,2- dioxide | Mitsunobu | Calc'd 376.0, found 376.0 |
| 87 | 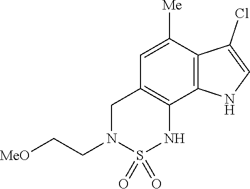 [SMILES: COCCN1Cc2cc(C)c3c(Cl)c[nH]c3c2NS1(=O)=O] | 7-chloro-3-(2- methoxyethyl)-6-methyl- 1,3,4,9-tetrahydro-[1,2,6] thiadiazino[4,3-g]indole 2,2- dioxide | Halide (Br) | Calc'd 328.0, found 328.0 |
| 89 | 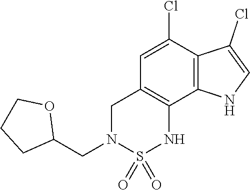 [SMILES: O=S1(=O)Nc2c(cc(Cl)c3c(Cl)c[nH]c23)CN1CC1CCCO1] | 6,7-dichloro-3- ((tetrahydrofuran-2-yl) methyl)-1,3,4,9-tetrahydro-[1,2,6] thiadiazino[4,3-g]indole 2,2- dioxide | Mesylate | Calc'd 376.0, found 376.0 |
| 94 | 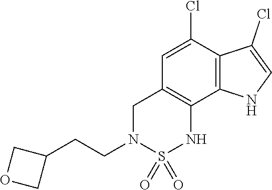 [SMILES: O=S1(=O)Nc2c(cc(Cl)c3c(Cl)c[nH]c23)CN1CCC1COC1] | 6,7-dichloro-3-(2- (oxetan-3-yl)ethyl)-1,3,4,9- tetrahydro-[1,2,6] thiadiazino[4,3-g]indole 2,2- dioxide | Mitsunobu | Calc'd 376., found 376.0 |
| 95 | 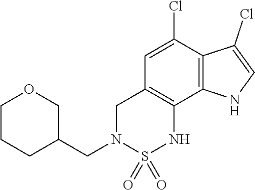 [SMILES: O=S1(=O)Nc2c(cc(Cl)c3c(Cl)c[nH]c23)CN1CC1CCCOC1] | 6,7-dichloro-3-((tetrahydro- 2H-pyran-3-yl)methyl)- 1,3,4,9-tetrahydro-[1,2,6] thiadiazino[4,3-g]indole 2,2- dioxide | Mitsunobu | Calc'd 390.0, found 390.0 |
| 96 | 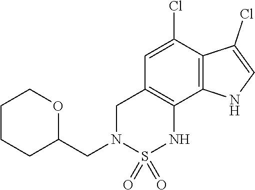 [SMILES: O=S1(=O)Nc2c(cc(Cl)c3c(Cl)c[nH]c23)CN1CC1CCCCO1] | 6,7-dichloro-3-((tetrahydro- 2H-pyran-2-yl)methyl)- 1,3,4,9-tetrahydro-[1,2,6] thiadiazino[4,3-g]indole 2,2- dioxide | Mesylate | Calc'd 388.0, found 388.0 |

### Example 8

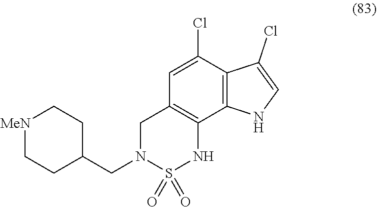 [SMILES: CN1CCC(CN2Cc3cc(Cl)c4c(Cl)c[nH]c4c3NS2(=O)=O)CC1]

### 6,7-dichloro-3-((1-methylpiperidin-4-yl)methyl)-1,3,4,9-tetrahydro-[1,2,6]thiadiazino[4,3-g]indole 2,2-dioxide (Compound 83)

Compound 83 was prepared in an analogous manner to that outlined in Scheme 6.

### Step 1. 1-allyl-6-chloro-8-nitro-3-(piperidin-4-ylmethyl)-3,4-dihydro-1H-benzo[c][1,2,6]thiadiazine 2,2-dioxide hydrochloride

The starting material tert-butyl 4-((1-allyl-6-chloro-8-nitro-2,2-dioxido-1,4-dihydro-3H-benzo[c][1,2,6]thiadiazin-3-yl)methyl)piperidine-1-carboxylate was prepared using the Mesylate/Halide alkylation method described in Examples 6 and 7 (compounds 18 and 34). tert-butyl 4-((1-allyl-6-chloro-8-nitro-2,2-dioxido-1,4-dihydro-3H-benzo[c][1,2,6]thiadiazin-3-yl)methyl)piperidine-1-carboxylate (250 mg, 0.499 mmol) was dissolved in HCl-dioxane (4 N, 10 mL) and stirred at room temperature for 2 h. The reaction mixture was concentrated to dryness to afford the desired product which was used without further purification. MS=401.1 (M+1).

### Step 2. 1-allyl-6-chloro-3-((1-methylpiperidin-4-yl)methyl)-8-nitro-3,4-dihydro-1H-benzo[c][1,2,6]thiadiazine 2,2-dioxide

To a solution of 1-allyl-6-chloro-8-nitro-3-(piperidin-4-ylmethyl)-3,4-dihydro-1H-benzo[c][1,2,6]thiadiazine 2,2-dioxide hydrochloride (140 mg, 0.32 mmol) in DMF (5 ml) was added potassium carbonate (88.4 mg, 0.64 mmol) at 0° C., the mixture was stirred at this temperature for 15 mins and then iodomethane (90.9 mmol, 0.64 mmol) was added. After that the mixture was warmed to room temperature and stirred for 2 h. Water (30 mL) was added and the mixture was extracted with EtOAc (3×20 ml). The combined organic phases were washed with brine, dried over Na₂SO₄, filtered and concentrated. The residue was purified by Prep-TLC (DCM/MeOH=15/1) to give 1-allyl-6-chloro-3-((1-methylpiperidin-4-yl)methyl)-8-nitro-3,4-dihydro-1H-benzo[c][1,2,6]thiadiazine 2,2-dioxide. MS=415.1 (M+1).

### Step 3. 1-allyl-6-chloro-3-((1-methylpiperidin-4-yl)methyl)-1,3,4,9-tetrahydro-[1,2,6]thiadiazino[4,3-g]indole 2,2-dioxide

1-allyl-6-chloro-3-((1-methylpiperidin-4-yl)methyl)-8-nitro-3,4-dihydro-1H-benzo[c][1,2,6]thiadiazine 2,2-dioxide was converted to 1-allyl-6-chloro-3-((1-methylpiperidin-4-yl)methyl)-1,3,4,9-tetrahydro-[1,2,6]thiadiazino[4,3-g]indole 2,2-dioxide following the procedure outlined in step 5 of Preparatory Example 2. MS=409.1 (M+1).

### Step 4. 1-allyl-6,7-dichloro-3-((1-methylpiperidin-4-yl)methyl)-1,3,4,9-tetrahydro-[1,2,6]thiadiazino[4,3-g]indole 2,2-dioxide

1-allyl-6-chloro-3-((1-methylpiperidin-4-yl)methyl)-1,3,4,9-tetrahydro-[1,2,6]thiadiazino[4,3-g]indole 2,2-dioxide was converted to 1-allyl-6,7-dichloro-3-((1-methylpiperidin-4-yl)methyl)-1,3,4,9-tetrahydro-[1,2,6]thiadiazino[4,3-g]indole 2,2-dioxide following the procedure outlined in step 6 of Preparatory Example 3. MS=443.1 (M+1).

### Step 5. 6,7-dichloro-3-((1-methylpiperidin-4-yl)methyl)-1,3,4,9-tetrahydro-[1,2,6]thiadiazino[4,3-g]indole 2,2-dioxide (83)

Deprotection Method B from Examples 1 and 2 (compounds 12 and 42) was used. ¹H NMR (400 MHz, CD₃OD): δ 7.30 (s, 1H), 6.85 (s, 1H), 4.70 (s, 2H), 2.89 (d, J=7.2 Hz, 2H), 2.78 (s, 2H), 2.72 (s, 3H), 2.00 (d, J=14.4 Hz, 4H), 1.46 (d, J=9.6 Hz, 4H). MS=403.0 (M+1).

### Example 9

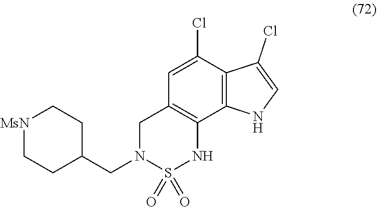

### 6,7-dichloro-3-((1-(methylsulfonyl)piperidin-4-yl)methyl)-1,3,4,9-tetrahydro-[1,2,6]thiadiazino[4,3-g]indole 2,2-dioxide (Compound 72)

Compound 72 was prepared in an analogous manner to that outlined in Scheme 7.

### Step 1. 1-allyl-6-chloro-3-((1-(methylsulfonyl)piperidin-4-yl)methyl)-8-nitro-3,4-dihydro-1H-benzo[c][1,2,6]thiadiazine 2,2-dioxide

The starting material 1-allyl-6-chloro-8-nitro-3-(piperidin-4-ylmethyl)-3,4-dihydro-1H-benzo[c][1,2,6]thiadiazine 2,2-dioxide hydrochloride was prepared as described in Example 8 (compound 83), step 1. To the stirred solution of 1-allyl-6-chloro-8-nitro-3-(piperidin-4-ylmethyl)-3,4-dihydro-1H-benzo[c][1,2,6]thiadiazine 2,2-dioxide hydrochloride (195 mg, 0.445 mmol) and triethylamine (180.0 mg, 1.78 mmol) in DCM (15 ml) at 0° C. under N₂ was added methanesulfonyl chloride (101.9 mg, 0.89 mmol). The mixture was warmed to room temperature and stirred at this temperature for 2 h. After the completion of reaction, the mixture was diluted with DCM (30 mL) and washed with water (15 mL) and brine (15 mL). After that the organic layer was dried over MgSO₄, filtered and concentrated. The product residue was used without further purification.

### Step 2. 1-allyl-6-chloro-3-((1-(methylsulfonyl)piperidin-4-yl)methyl)-1,3,4,9-tetrahydro-[1,2,6]thiadiazino[4,3-g]indole 2,2-dioxide

1-allyl-6-chloro-3-((1-(methylsulfonyl)piperidin-4-yl)methyl)-8-nitro-3,4-dihydro-1H-benzo[c][1,2,6]thiadiazine 2,2-dioxide was converted to 1-allyl-6-chloro-3-((1-(methylsulfonyl)piperidin-4-yl)methyl)-1,3,4,9-tetrahydro-[1,2,6]thiadiazino[4,3-g]indole 2,2-dioxide following the procedure outlined in step 5 of Preparatory Example 2. MS=471.0 (M-1).

### Step 3. 1-allyl-6,7-dichloro-3-((1-(methylsulfonyl)piperidin-4-yl)methyl)-1,3,4,9-tetrahydro-[1,2,6]thiadiazino[4,3-g]indole 2,2-dioxide

1-allyl-6-chloro-3-((1-(methylsulfonyl)piperidin-4-yl)methyl)-1,3,4,9-tetrahydro-[1,2,6]thiadiazino[4,3-g]indole 2,2-dioxide was converted to 1-allyl-6,7-dichloro-3-((1-(methylsulfonyl)piperidin-4-yl)methyl)-1,3,4,9-tetrahydro-[1,2,6]thiadiazino[4,3-g]indole 2,2-dioxide following the procedure outlined in step 6 of Preparatory Example 3.

### Step 4. 6,7-dichloro-3-((1-(methylsulfonyl)piperidin-4-yl)methyl)-1,3,4,9-tetrahydro-[1,2,6]thiadiazino[4,3-g]indole 2,2-dioxide (72)

Deprotection Method B from Examples 1 and 2 (compounds 12 and 42) was used. ¹H NMR (400 MHz, CD₃OD): δ 7.31 (s, 1H), 6.88 (s, 1H), 4.72 (s, 2H), 3.70-3.74 (d, 2H), 2.85-2.88 (d, 2H), 2.82 (s, 3H), 2.73-2.79 (t, 2H), 1.87-1.92 (d, 2H), 1.24-1.34 (m, 3H). MS=465.0 (M-H).

### Examples 10 and 11

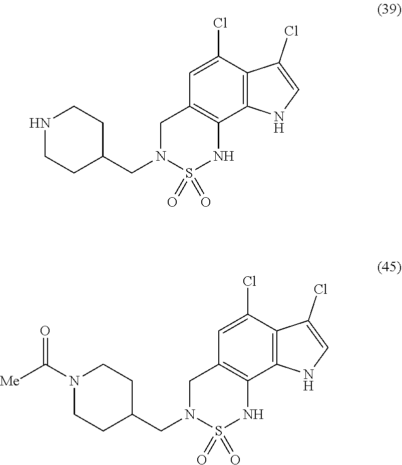 [SMILES: O=S1(=O)Nc2c(cc(Cl)c3c(Cl)c[nH]c23)CN1CC1CCNCC1 ; CC(=O)N1CCC(CN2Cc3cc(Cl)c4c(Cl)c[nH]c4c3NS2(=O)=O)CC1]

### 6,7-dichloro-3-(piperidin-4-ylmethyl)-1,3,4,9-tetrahydro-[1,2,6]thiadiazino[4,3-g]indole 2,2-dioxide (Compound 39) and 1-(4-((6,7-dichloro-2,2-dioxido-4,9-dihydro-[1,2,6]thiadiazino[4,3-g]indol-3(1H)-yl)methyl)piperidin-1-yl)ethan-1-one (Compound 45)

Compounds 39 and 45 were prepared in an analogous manner to that outlined in Scheme 8.

### Step 1. tert-butyl 4-((1-allyl-6-chloro-2,2-dioxido-4,9-dihydro-[1,2,6]thiadiazino[4,3-g]indol-3(1H)-yl)methyl)piperidine-1-carboxylate

The starting material tert-butyl 4-((1-allyl-6-chloro-8-nitro-2,2-dioxido-1,4-dihydro-3H-benzo[c][1,2,6]thiadiazin-3-yl)methyl)piperidine-1-carboxylate was prepared using the Mesylate/Halide alkylation method described in Examples 6 and 7 (compounds 18 and 34). tert-butyl 4-((1-allyl-6-chloro-8-nitro-2,2-dioxido-1,4-dihydro-3H-benzo[c][1,2,6]thiadiazin-3-yl)methyl)piperidine-1-carboxylate was converted to tert-butyl 4-((1-allyl-6-chloro-2,2-dioxido-4,9-dihydro-[1,2,6]thiadiazino[4,3-g]indol-3(1H)-yl)methyl)piperidine-1-carboxylate following the procedure outlined in step 5 of Preparatory Example 2. MS=395.1 (M-C₄H₈—CO₂+1).

### Step 2. tert-butyl 4-((1-allyl-6,7-dichloro-2,2-dioxido-4,9-dihydro-[1,2,6]thiadiazino[4,3-g]indol-3(1H)-yl)methyl)piperidine-1-carboxylate

tert-butyl 4-((1-allyl-6-chloro-2,2-dioxido-4,9-dihydro-[1,2,6]thiadiazino[4,3-g]indol-3(1H)-yl)methyl)piperidine-1-carboxylate was converted to tert-butyl 4-((1-allyl-6,7-dichloro-2,2-dioxido-4,9-dihydro-[1,2,6]thiadiazino[4,3-g]indol-3(1H)-yl)methyl)piperidine-1-carboxylate following the procedure outlined in step 6 of Preparatory Example 3.

### Step 3. tert-butyl 4-((6,7-dichloro-2,2-dioxido-4,9-dihydro-[1,2,6]thiadiazino[4,3-g]indol-3(1H)-yl)methyl)piperidine-1-carboxylate

The titled compound was made utilizing Deprotection Method A from Examples 1 and 2 (compounds 12 and 42).

### Step 4. 6,7-dichloro-3-(piperidin-4-ylmethyl)-1,3,4,9-tetrahydro-[1,2,6]thiadiazino[4,3-g]indole 2,2-dioxide (39)

tert-butyl 4-((6,7-dichloro-2,2-dioxido-4,9-dihydro-[1,2,6]thiadiazino[4,3-g]indol-3(1H)-yl)methyl)piperidine-1-carboxylate (57 mg, 0.12 mmol) was dissolved in HCl/1,4-Dioxane (6 mL, 4 N) and stirred at 25° C. After 2 h, the reaction mixture was concentrated and purified by HPLC to give 6,7-dichloro-3-(piperidin-4-ylmethyl)-1,3,4,9-tetrahydro-[1,2,6]thiadiazino[4,3-g]indole 2,2-dioxide (39). ¹H NMR (400 MHz, CD₃OD): δ 7.27 (s, 1H), 6.81 (s, 1H), 4.67 (s, 2H), 3.39 (d, J=12.8 Hz, 2H), 2.94-3.02 (m, 2H), 2.87 (d, J=6.8 Hz, 2H), 1.98-2.06 (m, 3H), 1.30-1.53 (m, 2H). MS=389.0 (M+1).

### Step 5. 1-(4-((6,7-dichloro-2,2-dioxido-4,9-dihydro-[1,2,6]thiadiazino[4,3-g]indol-3(1H)-yl)methyl)piperidin-1-yl)ethan-1-one (45)

A solution of acetic acid (5.9 mg, 0.1 mmol), TEA (4.0 equiv), EDCI (2.0 equiv) and HOBt (2.0 equiv) in DCM (5 mL) was stirred at 0° C. for 15 min. Then 6,7-dichloro-3-(piperidin-4-ylmethyl)-1,3,4,9-tetrahydro-[1,2,6]thiadiazino[4,3-g]indole 2,2-dioxide (19.1 mg, 0.05 mmol) was added and the mixture stirred overnight at 25° C. The reaction mixture was quenched by the addition of 50 mL of water. The resulting mixture was extracted with 3×25 mL EtOAc. The combined organics were rinsed with 20 mL of brine, dried over Na₂SO₄, filtered, concentrated and purified by Prep-HPLC to give 1-(4-((6,7-dichloro-2,2-dioxido-4,9-dihydro-[1,2,6]thiadiazino[4,3-g]indol-3(1H)-yl)methyl)piperidin-1-yl)ethan-1-one (45). ¹H NMR (400 MHz, DMSO-d6): δ 11.25 (s, 1H), 10.06 (s, 1H), 7.58 (s, 1H), 6.94 (s, 1H), 4.68 (s, 2H), 4.35 (d, J=13.2 Hz, 1H), 3.81 (d, J=13.2 Hz, 1H), 3.01 (t, J=11.6 Hz, 1H), 2.75 (d, J=7.2 Hz, 2H), 2.51-2.56 (m, 1H), 1.98 (s, 3H), 1.83-1.95 (m, 1H), 1.64-1.77 (m, 2H), 1.02-1.18 (m, 1H), 0.81-1.02 (m, 1H). MS=431.0 (M+1).

TABLE 6
The following compounds were prepared using procedures similar to those
described for Examples 10 and 11 (compounds 39 and 45) using the appropriate starting materials.

| Example | | | Exact Mass |
|---|---|---|---|
| No. | Structure | IUPAC Name | [M + H]⁺ |
| 28 | 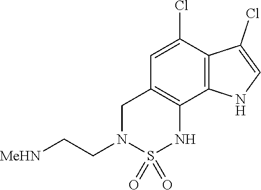 [SMILES: CNCCN1Cc2cc(Cl)c3c(Cl)c[nH]c3c2NS1(=O)=O] | 6,7-dichloro-3-(2-(methylamino)ethyl)- 1,3,4,9-tetrahydro-[1,2,6] thiadiazino[4,3-g]indole 2,2- dioxide | Calc'd 349.0, found 349.0 |
| 47 | 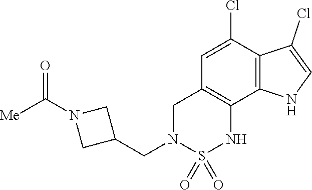 [SMILES: CC(=O)N1CC(CN2Cc3cc(Cl)c4c(Cl)c[nH]c4c3NS2(=O)=O)C1] | 1-(3-((6,7-dichloro-2,2-dioxido-4,9- dihydro-[1,2,6]thiadiazino[4,3-g] indol-3(1H)-yl)methyl)azetidin-1-yl) ethan-1-one | Calc'd 403.0, found 403.0 |
| 90 | 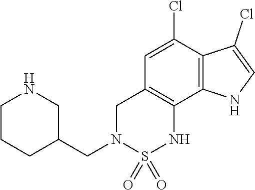 [SMILES: O=S1(=O)Nc2c(cc(Cl)c3c(Cl)c[nH]c23)CN1CC1CCCNC1] | 6,7-dichloro-3-(piperidin-3-ylmethyl)- 1,3,4,9-tetrahydro-[1,2,6] thiadiazino[4,3-g]indole 2,2- dioxide | Calc'd 389.0, found 389.0 |
| 91 | 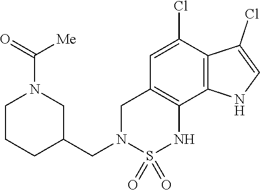 [SMILES: CC(=O)N1CCCC(CN2Cc3cc(Cl)c4c(Cl)c[nH]c4c3NS2(=O)=O)C1] | 1-(3-((6,7-dichloro-2,2-dioxido-4,9- dihydro-[1,2,6]thiadiazino[4,3-g] indol-3(1H)-yl)methyl)piperidin-1-yl) ethan-1-one | Calc'd 431.0, found 431.0 |

### Example 12

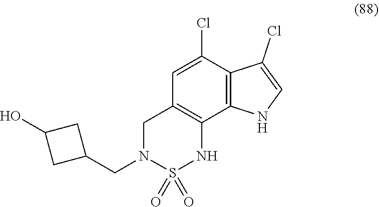 [SMILES: O=S1(=O)Nc2c(cc(Cl)c3c(Cl)c[nH]c23)CN1CC1CC(O)C1]

### 6,7-dichloro-3-((3-hydroxycyclobutyl)methyl)-1,3,4,9-tetrahydro-[1,2,6]thiadiazino[4,3-g]indole 2,2-dioxide (Compound 88)

Compound 88 was prepared in an analogous manner to that outlined in Scheme 9.

### Step 1. 1-allyl-6-chloro-3-((3-hydroxycyclobutyl)methyl)-1,3,4,9-tetrahydro-[1,2,6]thiadiazino[4,3-g]indole 2,2-dioxide

The starting material 1-allyl-3-((3-((tert-butyldiphenylsilyl)oxy)cyclobutyl)methyl)-6-chloro-1,3,4,9-tetrahydro-[1,2,6]thiadiazino[4,3-g]indole 2,2-dioxide was synthesized in a similar fashion to Examples 6 and 7 (compounds 18 and 34) using step 1, Mitsunobu alkylation and step 2. To a mixture of 1-allyl-3-((3-((tert-butyldiphenylsilyl)oxy)cyclobutyl)methyl)-6-chloro-1,3,4,9-tetrahydro-[1,2,6]thiadiazino[4,3-g]indole 2,2-dioxide (39.0 mg, 0.06 mmol) in THF (5 mL) was added 1 M TBAF in THF (310 μL, 0.31 mmol), and the mixture was stirred for 1 h at room temperature under N₂ and then quenched with water (10 mL) and extracted with EtOAc (3×10 mL). The organic phase was washed with brine (10 mL), dried over Na₂SO₄, filtered and the filtrate was concentrated. The product residue was used without further purification.

### Step 2. 6-chloro-3-((3-hydroxycyclobutyl)methyl)-1,3,4,9-tetrahydro-[1,2,6]thiadiazino[4,3-g]indole 2,2-dioxide

The unpurified 1-allyl-6-chloro-3-((3-hydroxycyclobutyl)methyl)-1,3,4,9-tetrahydro-[1,2,6]thiadiazino[4,3-g]indole 2,2-dioxide was dissolved in THF (5 mL) and cooled to 0° C., then NaBH₄ (4.8 mg, 0.13 mol), Pd(PPh₃)₄(7.3 mg, 0.006 mmol) was added. The resulting mixture was warmed to room temperature and stirred for 2 h under N₂. The reaction mixture was quenched with water (10 mL) and extracted with EtOAc (3×10 mL). The organic phase was washed with brine (10 mL), dried over Na₂SO₄, filtered and the filtrate was concentrated. The residue was purified by Prep-HPLC to give 6-chloro-3-((3-hydroxycyclobutyl)methyl)-1,3,4,9-tetrahydro-[1,2,6]thiadiazino[4,3-g]indole 2,2-dioxide.

### Step 3. 6,7-dichloro-3-((3-hydroxycyclobutyl)methyl)-1,3,4,9-tetrahydro-[1,2,6]thiadiazino[4,3-g]indole 2,2-dioxide (88)

To a mixture of 6-chloro-3-((3-hydroxycyclobutyl)methyl)-1,3,4,9-tetrahydro-[1,2,6]thiadiazino[4,3-g]indole 2,2-dioxide (7 mg, 0.020 mmol) in THF (5 mL) and DMF (0.5 mL) was added NCS (2.7 mg, 0.020 mmol) at 0° C. under N2. The resulting mixture was warmed to room temperature and stirred for 18 h, quenched with water (10 mL) and extracted with EtOAc (3×10 mL). The organic phase was washed with brine (10 mL), dried over Na₂SO₄, filtered and the filtrate was concentrated. The residue was purified by Prep-TLC (DCM/MeOH=10/1) to give 6,7-dichloro-3-((3-hydroxycyclobutyl)methyl)-1,3,4,9-tetrahydro-[1,2,6]thiadiazino[4,3-g]indole 2,2-dioxide (88) as ˜10:1 mixture of cis:trans isomers. ¹H NMR (400 MHz, CD₃OD): δ 7.30 (s, 1H), 6.86 (s, 1H), 4.68 (s, 2H), 4.03-4.11 (m, 1H), 3.00 (t, J=6.8 Hz, 2H), 2.39-2.45 (m, 2H), 2.06-2.14 (m, 1H), 1.56-1.64 (m, 2H). MS=376.0 (M+1).

TABLE 7
The following compounds were prepared using procedures similar to those
described for Example 12 (compound 88) using the appropriate starting materials.

| Compd. | | | Exact Mass |
|---|---|---|---|
| No. | Structure | IUPAC | [M + H]⁺ |
| 24 | 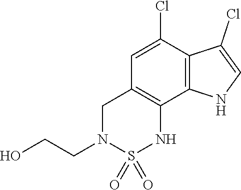 [SMILES: O=S1(=O)Nc2c(cc(Cl)c3c(Cl)c[nH]c23)CN1CCO] | 6,7-dichloro-3-(2-hydroxyethyl)- 1,3,4,9-tetrahydro-[1,2,6] thiadiazino[4,3-g]indole 2,2- dioxide | Calc'd 335.99, found 336.0 |

### Example 13

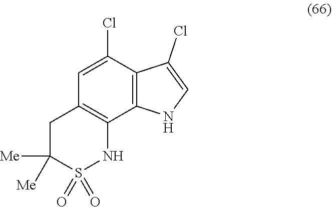 [SMILES: CC1(C)Cc2cc(Cl)c3c(Cl)c[nH]c3c2NS1(=O)=O]

### 6,7-dichloro-3,3-dimethyl-1,3,4,9-tetrahydro-[1,2]thiazino[4,3-g]indole 2,2-dioxide (Compound 66)

Compound 66 was prepared in an analogous manner to that outlined in Scheme 10.

### Step 1. methyl 5-chloro-2-(methylsulfonamido)benzoate

A solution of Benzoic acid, 2-amino-5-chloro-, methyl ester (55 g, 296 mmol) and pyridine (71.6 mL, 889 mmol) in DCM (500 mL) under an inert atmosphere of nitrogen and cooled to 0° C. was treated with methanesulfonyl chloride (25.2 mL, 326 mmol). The resulting solution was stirred at 0° C. for 20 min. Then the ice bath was removed and the reaction allowed to stir at room temperature for 2 h. The reaction was concentrated to dryness, poured into 400 mL of 3 N aqueous HCl and extracted with and extracted with EtOAc (3×200 mL). The combined organic layers were washed with brine (200 mL), dried (MgSO₄), filtered, and concentrated under reduced pressure to afford methyl 5-chloro-2-(methylsulfonamido)benzoate which was used without further purification. ¹H NMR (400 MHz, CDCl₃): δ 10.33 (s, 1H), 8.03 (d, J=2.5 Hz, 1H), 7.72 (d, J=9.0 Hz, 1H), 7.51 (dd, J=8.9, 2.5 Hz, 1H), 3.96 (s, 3H), 3.06 (s, 3H). MS=261.9 (M−1).

### Step 2. methyl 2-(N-allylmethylsulfonamido)-5-chlorobenzoate

To a solution of methyl 5-chloro-2-(methylsulfonamido)benzoate (66.9 g, 253.7 mmol) in dry DMF (600 mL) was added potassium carbonate (105.2 g, 761.1 mmol) at 10° C., followed by allyl bromide (46.0 g, 380.6 mmol). The mixture was stirred at 25° C. overnight. The reaction mixture was poured into water, and the product was extracted with EtOAc, washed with brine, dried (Na₂SO₄), and concentrated to dryness. The product methyl 2-[allyl(methylsulfonyl)amino]-5-chloro-benzoate was used without further purification. Purification can be achieved by chromatography on silica using 10-25% EtOAc/hexanes. ¹H NMR (400 MHz, CDCl₃): δ 7.90 (d, J=2.5 Hz, 1H), 7.50 (dd, J=8.5, 2.6 Hz, 1H), 7.32 (d, J=8.5 Hz, 1H), 5.97-5.79 (m, 1H), 5.09 (dd, J=20.9, 5.1 Hz, 2H), 4.22 (s, 2H), 3.92 (s, 2H), 2.98 (s, 2H). MS=303.9 (M+1).

### Step 3. 1-allyl-6-chloro-1H-benzo[c][1,2]thiazin-4(3H)-one 2,2-dioxide

Methyl 2-(N-allylmethylsulfonamido)-5-chlorobenzoate (15.6 g, 51.4 mmol) was added to a stirred suspension of NaH (4.11 g, 102.7 mmol) in DMF (50 mL) at 25° C. The mixture was stirred at 25° C. for 2 h. Then the reaction mixture was acidified with 1 N HCl (aq, 150 mL) and extracted with EtOAc (3×50 mL). The combined extracts were washed with water (100 mL), washed with brine (100 mL), dried over Na₂SO₄, filtered and concentrated to give 1-allyl-6-chloro-1H-benzo[c][1,2]thiazin-4(3H)-one 2,2-dioxide. ¹H NMR (400 MHz, CDCl₃): δ 8.09 (d, J=2.5 Hz, 1H), 7.57 (dd, J=8.8, 2.6 Hz, 1H), 7.17 (d, J=8.8 Hz, 1H), 5.93 (ddt, J=17.1, 10.2, 5.1 Hz, 1H), 5.39 (dd, J=28.7, 13.8 Hz, 2H), 4.53-4.63 (m, 2H), 4.32 (s, 2H). MS=269.9 (M−1).

### Step 4. 1-allyl-6-chloro-3,3-dimethyl-1H-benzo[c][1,2]thiazin-4(3H)-one 2,2-dioxide

To a solution of 1-allyl-6-chloro-1H-benzo[c][1,2]thiazin-4(3H)-one 2,2-dioxide (4.0 g, 14.7 mmol) in DMF (30 mL) at 0° C. was added sequentially potassium carbonate (4.48 g, 32.4 mmol) and iodomethane (2.75 mL, 44.2 mmol). The reaction was stirred at 25° C. for 12 h. The reaction was evaporated and diluted with 50 ml of EtOAc, which was washed with brine (50 mL). The organic layer was dried over anhydrous sodium sulfate. After concentration under vacuum, the resulting solidi was recrystallized from hexane to afford 1-allyl-6-chloro-3,3-dimethyl-1H-benzo[c][1,2]thiazin-4(3H)-one 2,2-dioxide. ¹H NMR (400 MHz, CDCl₃): δ 8.09 (d, J=2.6 Hz, 1H), 7.52 (dd, J=8.8, 2.6 Hz, 1H), 7.10 (d, J=8.8 Hz, 1H), 6.04-5.87 (m, 1H), 5.48 (d, J=17.2 Hz, 1H), 5.37 (d, J=10.5 Hz, 1H), 4.57 (dd, J=3.0, 1.8 Hz, 2H), 1.63 (s, 6H).

### Step 5. 1-allyl-6-chloro-4-hydroxy-3,3-dimethyl-3,4-dihydro-1H-benzo[c][1,2]thiazine 2,2-dioxide

To a solution of 1-allyl-6-chloro-3,3-dimethyl-1H-benzo[c][1,2]thiazin-4(3H)-one 2,2-dioxide (4.1 g, 13.68 mmol) in methanol (30 mL) at 0° C. was added sodium borohydride (569.2 mg, 15.0 mmol). The reaction was stirred at 25° C. for 1 h. The reaction was evaporated and diluted with 50 ml of EtOAc, which was washed with brine (25 mL). The organic layer was dried over anhydrous sodium sulfate. Purification by silica gel column chromatography (20% EtOAc in hexane) afforded 1-allyl-6-chloro-4-hydroxy-3,3-dimethyl-3,4-dihydro-1H-benzo[c][1,2]thiazine 2,2-dioxide. ¹H NMR (400 MHz, CD₃OD): δ 7.64-7.59 (m, 1H), 7.21-7.34 (m, 1H), 6.98 (d, J=8.8 Hz, 1H), 5.98 (ddt, J=17.2, 10.4, 4.5 Hz, 1H), 5.41 (dd, J=17.2, 1.2 Hz, 1H), 5.27 (dd, J=10.5, 1.3 Hz, 1H), 4.67-4.48 (m, 1H), 4.34-4.19 (m, 1H), 3.30 (d, J=1.6 Hz, 2H), 1.53 (s, 3H), 1.20 (s, 3H). MS=336.0 (M+Cl)⁻.

### Step 6. 1-allyl-6-chloro-3,3-dimethyl-3,4-dihydro-1H-benzo[c][1,2]thiazine 2,2-dioxide

To a solution of 1-allyl-6-chloro-4-hydroxy-3,3-dimethyl-3,4-dihydro-1H-benzo[c][1,2]thiazine 2,2-dioxide (3.5 g, 11.6 mmol) in TFA (20 mL) at 0° C. was added triethylsilane (10 mL). The mixture was stirred at 25° C. for 16 h. The mixture was diluted with EtOAc (60 mL) and carefully adjusted to pH to 6-7 with saturated aqueous NaHCO₃ solution. The organic layer was separated and dried over Na₂SO₄, filtered, concentrated and purified by column chromatography on silica gel (PE:EA=5:1 to 1:1) to give 1-allyl-6-chloro-3,3-dimethyl-3,4-dihydro-1H-benzo[c][1,2]thiazine 2,2-dioxide. ¹H NMR (400 MHz, CDCl₃): δ 7.17 (dd, J=8.8, 2.4 Hz, 1H), 7.09 (d, J=2.2 Hz, 1H), 6.87 (d, J=8.8 Hz, 1H), 5.86-6.03 (m, 1H), 5.44 (d, J=17.2 Hz, 1H), 5.30 (d, J=10.4 Hz, 1H), 4.42 (dd, J=4.2, 2.1 Hz, 2H), 3.22 (s, 2H), 1.46 (s, 6H).

### Step 7. 1-allyl-6-chloro-3,3-dimethyl-8-nitro-3,4-dihydro-1H-benzo[c][1,2]thiazine 2,2-dioxide

To a solution of 1-allyl-6-chloro-3,3-dimethyl-3,4-dihydro-1H-benzo[c][1,2]thiazine 2,2-dioxide (260.mg, 0.91 mmol) and potassium nitrate (570 mg, 5.6 mmol) in acetonitrile (10 mL) at 0° C. was carefully added trifluoroacetic anhydride (210 mg, 1 mmol). The mixture was stirred at 0° C. for 2 h. The reaction mixture was slowly treated with saturated aqueous NaHCO₃ solution and then diluted with EtOAc (20 mL). The organic layer was separated, dried over Na₂SO₄, filtered, concentrated and purified by column chromatography silica gel (PE:EA=5:1) to give 1-allyl-6-chloro-3,3-dimethyl-8-nitro-3,4-dihydro-1H-benzo[c][1,2]thiazine 2,2-dioxide. ¹H NMR (400 MHz, CDCl₃): δ 7.75 (d, J=2.4 Hz, 1H), 7.34 (d, J=2.4 Hz, 1H), 5.61 (ddt, J=16.9, 10.1, 6.7 Hz, 1H), 5.12-5.27 (m, 2H), 4.34 (d, J=6.7 Hz, 2H), 3.23 (s, 2H), 1.51 (s, 6H).

### Step 8. 1-allyl-6-chloro-3,3-dimethyl-1,3,4,9-tetrahydro-[1,2]thiazino[4,3-g]indole 2,2-dioxide

A solution of 1-allyl-6-chloro-3,3-dimethyl-8-nitro-3,4-dihydro-1H-benzo[c][1,2]thiazine 2,2-dioxide (247 mg, 0.75 mmol) in THF (20 mL) at −40° C. under N₂ atmosphere was treated with vinylmagnesium bromide solution (4.5 mL, 4.5 mmol, ˜1.0 M in THF) by dropwise addition over 5 min. After 1 h, the reaction mixture was quenched with an aqueous saturated NH₄Cl solution (10 mL). The product was extracted with EtOAc (3×20 mL). The combined organic layer was dried over MgSO₄. After filtration and concentration, the crude product was purified by silica gel column chromatography (EtOAc/PE=1/5) to give 1-allyl-6-chloro-3,3-dimethyl-1,3,4,9-tetrahydro-[1,2]thiazino[4,3-g]indole 2,2-dioxide. ¹H NMR (400 MHz, CDCl₃) δ 8.79 (s, 1H), 7.16 (s, 1H), 6.86 (s, 1H), 6.61 (s, 1H), 6.35 (d, J=13.7 Hz, 1H), 5.92 (d, J=17.1 Hz, 1H), 5.63 (d, J=10.7 Hz, 1H), 4.41 (s, 2H), 1.53 (s, 6H).

### Step 9. 1-allyl-6,7-dichloro-3,3-dimethyl-1,3,4,9-tetrahydro-[1,2]thiazino[4,3-g]indole 2,2-dioxide

A solution of 1-allyl-6-chloro-3,3-dimethyl-1,3,4,9-tetrahydro-[1,2]thiazino[4,3-g]indole 2,2-dioxide (75 mg, 0.23 mmol) in THF (5 mL)/DMF (0.1 mL) at −10° C. was treated with N-chlorosuccinimide (40.1 mg, 0.3 mmol). The reaction was allowed to warm to 25° C. and left to stir overnight. Aqueous saturated Na₂S₂O₃ solution (5 mL) was added and the resulting mixture left to stir for 10 min. The mixture was concentrated, water (50 mL) was added, and the product was extracted with 3×10 mL EtOAc. The combined organics were rinsed with 30 mL of brine, dried over Na₂SO₄, filtered, concentrated and purified by column chromatography (EtOAc/PE=1/6 to 1/3) to afford 1-allyl-6,7-dichloro-3,3-dimethyl-1,3,4,9-tetrahydro-[1,2]thiazino[4,3-g]indole 2,2-dioxide. MS=357.0 (M−1).

### Step 10. 6,7-dichloro-3,3-dimethyl-1,3,4,9-tetrahydro-[1,2]thiazino[4,3-g]indole 2,2-dioxide (66)

To a solution of 1-allyl-6,7-dichloro-3,3-dimethyl-1,3,4,9-tetrahydro-[1,2]thiazino[4,3-g]indole 2,2-dioxide (60 mg, 0.17 mmol) in THF (5 mL) was added sodium borohydride (12.6 mg, 0.33 mmol) and Pd(PPh₃)₄(0.05 eq.). The mixture was stirred at 20° C. for 12 h. Then H₂O (10 mL) was added to quench the reaction, and the product was extracted with EtOAc (3×10 mL), washed with brine (10 mL), separated, dried over anhydrous Na₂SO₄, and filtered. The filtrate was concentrated and purified by Prep-HPLC to afford 6,7-dichloro-3,3-dimethyl-1,3,4,9-tetrahydro-[1,2]thiazino[4,3-g]indole 2,2-dioxide (66). ¹H NMR (400 MHz, CD₃OD): δ 7.27 (s, 1H), 6.86 (s, 1H), 3.26 (s, 2H), 1.46 (s, 6H). MS=317.0 (M−1).

### Example 14

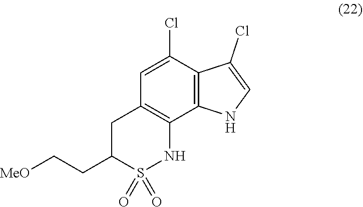 [SMILES: COCCC1Cc2cc(Cl)c3c(Cl)c[nH]c3c2NS1(=O)=O]

6,7-dichloro-3-(2-methoxyethyl)-1,3,4,9-tetrahydro-[1,2]thiazino[4,3-g]indole 2,2-dioxide (Compound 22) Compound 22 was prepared in an analogous manner to that outlined in Scheme 11.

### Step 1. 1-allyl-6-chloro-4-hydroxy-3,4-dihydro-1H-benzo[c][1,2]thiazine 2,2-dioxide

A solution of 1-allyl-6-chloro-1H-benzo[c][1,2]thiazin-4(3H)-one 2,2-dioxide (1800 mg, 6.62 mmol) in Methanol (29 mL) at 0° C. was treated with sodium borohydride (250.6 mg, 6.62 mmol) and stirred until complete by LCMS. Volatiles were removed by concentration under reduced pressure. The left over residue was solubilized with EtOAc and poured into 30 mL of saturated NH₄Cl and extracted with 3×20 mL EtOAc. The combined organics were rinsed with 15 mL of brine, dried with MgSO₄, filtered, and concentrated to dryness. The product used without further purification. MS=273.9 (M+1).

### Step 2. 1-allyl-6-chloro-3,4-dihydro-1H-benzo[c][1,2]thiazine 2,2-dioxide

A solution of 1-allyl-6-chloro-4-hydroxy-3,4-dihydro-1H-benzo[c][1,2]thiazine 2,2-dioxide (1.8 g, 6.62 mmol) in TFA (37 mL) was treated with triethylsilane (18.5 mL) and stirred at room temperature for 18 h. Volatiles were removed by concentration multiple times from toluene. The concentrated residue was purified without further manipulation. Purification was achieved by chromatography on silica using 5-20% EtOAc/Hex. MS=257.9 (M+1).

### Step 3. 1-allyl-6-chloro-8-nitro-3,4-dihydro-1H-benzo[c][1,2]thiazine 2,2-dioxide

Potassium nitrate (403.0 mg, 3.99 mmol) was added to a solution of 1-allyl-6-chloro-3,4-dihydro-1H-benzo[c][1,2]thiazine 2,2-dioxide (934 mg, 3.62 mmol) in a mixture of TFAA (3.1 mL, 22.5 mmol) and acetonitrile (24 mL) at 0° C. After 2 hours the 0° C. reaction mixture was slowly quenched with saturated aqueous NaHCO₃ until the reaction mixture was basic. The reaction mixture was poured into 30 mL of EtOAc. The organic layer was separated and the aqueous layer extracted further with 2×20 mL EtOAc. The combined organics were rinsed with 15 mL of brine, dried with MgSO₄, filtered, and concentrated to dryness. The product used without further purification. MS=302.8 (M+1).

### Step 4. 1-allyl-6-chloro-1,3,4,9-tetrahydro-[1,2]thiazino[4,3-g]indole 2,2-dioxide

The titled compound was made in an analogous process as described in Preparatory Example 2, Step 5. Purification was achieved by chromatography on silica using 5-25% EtOAc/Hex. MS=296.9 (M+1).

### Step 5. 1-allyl-6,7-dichloro-1,3,4,9-tetrahydro-[1,2]thiazino[4,3-g]indole 2,2-dioxide

A solution of 1-allyl-6-chloro-1,3,4,9-tetrahydro-[1,2]thiazino[4,3-g]indole 2,2-dioxide (154 mg, 0.52 mmol) in DMF (2.0 mL) was treated with NCS (69.2 mg, 0.52 mmol) and stirred at 90° C. for 1 h. The reaction mixture was poured into 30 mL of water and extracted with 3×15 mL TBME. The combined organics were rinsed with 10 mL of brine, dried with MgSO₄, filtered, and concentrated to dryness. Purification was achieved by chromatography on silica using 10-30% EtOAc/Hex. MS=328.8 (M−1).

### Step 6. 1-allyl-6,7-dichloro-9-((2-(trimethylsilyl)ethoxy)methyl)-1,3,4,9-tetrahydro-[1,2]thiazino[4,3-g]indole 2,2-dioxide

A solution of 1-allyl-6,7-dichloro-1,3,4,9-tetrahydro-[1,2]thiazino[4,3-g]indole 2,2-dioxide (163.4 mg, 0.49 mmol) in DMF (1.5 mL) at 25 C was treated with sodium hydride (60%) (23.7 mg, 0.59 mmol) and stirred at 25° C. for 20 min. At this time, 2-(trimethylsilyl)ethoxymethyl chloride (0.11 mL, 0.64 mmol) was added and the reaction stirred for 30 min. LCMS shows clean conversion to the desired product. The reaction mixture was poured into 30 mL of water and extracted with 3×15 mL TBME. The combined organics were rinsed with 10 mL of brine, dried with MgSO₄, filtered, and concentrated to dryness. Purification was achieved by chromatography on silica using 5-20% EtOAc/Hex. MS=482.8 (M+23).

### Step 7. 1-allyl-6,7-dichloro-3-(2-methoxyethyl)-9-((2-(trimethylsilyl)ethoxy)methyl)-1,3,4,9-tetrahydro-[1,2]thiazino[4,3-g]indole 2,2-dioxide

A solution of 1-allyl-6,7-dichloro-9-((2-(trimethylsilyl)ethoxy)methyl)-1,3,4,9-tetrahydro-[1,2]thiazino[4,3-g]indole 2,2-dioxide (180.8 mg, 0.39 mmol) in tetrahydrofuran (1.6 mL) was treated with lithium hexamethyldisilazide (0.45 mL of a 1.0 M solution in tetrahydrofuran, 0.45 mmol) at −78° C. After 3 h, 1-bromo-2-methoxyethane (77 μL, 0.82 mmol) was added and the reaction mixture was allowed to warm to room temperature. After 3 h, the reaction mixture was quenched by the addition of aqueous saturated NH₄Cl (2 mL) at −78° C. The dry ice bath was removed and the reaction mixture allowed to warm to room temperature. The reaction mixture was poured into 30 mL of water and extracted with 3×15 mL TBME. Purification was achieved by chromatography on silica using 5-25% EtOAc/Hex. MS=540.8 (M+23).

### Step 8. 1-allyl-6,7-dichloro-3-(2-methoxyethyl)-1,3,4,9-tetrahydro-[1,2]thiazino[4,3-g]indole 2,2-dioxide

A solution of 1-allyl-6,7-dichloro-3-(2-methoxyethyl)-9-((2-(trimethylsilyl)ethoxy)methyl)-1,3,4,9-tetrahydro-[1,2]thiazino[4,3-g]indole 2,2-dioxide (35.9 mg, 0.069 mmol) in DMF (0.5 mL) was treated sequentially with ethylene diamine (28 μL, 0.42 mmol) and TBAF (210 μL, 0.21 mmol, ˜1.0 M in THF). The resulting mixture was heated to 80° C. for 12 h. The reaction mixture was poured into water and extracted with 3×15 mL TBME. The combined organics were rinsed successively with 10 mL of aqueous saturated KHSO₄ and 10 mL of brine, dried with MgSO₄, filtered, and concentrated to dryness. Purification was achieved by chromatography on silica using 10-30% EtOAc/Hex. MS=388.8 (M+1).

### Step 9. 6,7-dichloro-3-(2-methoxyethyl)-1,3,4,9-tetrahydro-[1,2]thiazino[4,3-g]indole 2,2-dioxide

A solution of 1-allyl-6,7-dichloro-3-(2-methoxyethyl)-1,3,4,9-tetrahydro-[1,2]thiazino[4,3-g]indole 2,2-dioxide (35.9 mg, 0.069 mmol) in THF (0.75 mL) was sparged with nitrogen for 1 min and then treated sequentially, under continuous nitrogen stream, with sodium borohydride (2.5 mg, 0.065 mmol) and tetrakis(triphenylphosphine)palladium(0) (3.8 mg, 0.003 mmol). The reaction mixture was stirred at room temperature for about 6 hours. The reaction mixture was poured into water and extracted with 3×15 mL TBME. The combined organics were rinsed successively with 10 mL of brine, dried with MgSO₄, filtered, and concentrated to dryness to furnish the racemic compound 22. Purification was achieved by chromatography on silica using 30-60% EtOAc/Hex. ¹H NMR (400 MHz, CDCl₃) δ 8.83 (s, 1H), 7.14 (d, J=2.7 Hz, 1H), 7.10 (s, 1H), 6.88 (s, 1H), 3.68-3.50 (m, 4H), 3.35 (s, 4H), 2.39 (ddt, J=14.7, 7.4, 4.9 Hz, 1H), 1.85 (dtd, J=14.6, 6.9, 4.6 Hz, 1H). MS=346.8 (M−1).

TABLE 7
The following compounds were prepared using procedures similar to those
described for Example 14 (compound 22) using the appropriate starting materials.

| Compd | | | Exact Mass |
|---|---|---|---|
| No. | Structure | IUPAC Name | [M − H]⁻ |
| 16 | 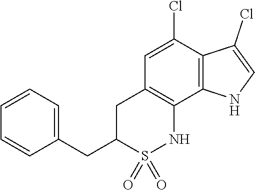 [SMILES: O=S1(=O)Nc2c(cc(Cl)c3c(Cl)c[nH]c23)CC1Cc1ccccc1] | 3-benzyl-6,7-dichloro- 1,3,4,9-tetrahydro-[1,2] thiazino[4,3-g]indole 2,2- dioxide | Calc'd 379.0, found 378.8 |

### Example 15

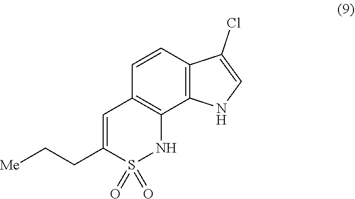 [SMILES: CCCC1=Cc2ccc3c(Cl)c[nH]c3c2NS1(=O)=O]

### 7-chloro-3-propyl-1,9-dihydro-[1,2]thiazino[4,3-g]indole 2,2-dioxide (Compound 9)

Compound 9 was prepared in an analogous manner to that outlined in Scheme 12.

### Step 1. 1-allyl-8-nitro-3-propyl-1H-benzo[c][1,2]thiazine 2,2-dioxide

A solution of 2-fluoro-3-nitrobenzaldehyde (500 mg, 2.96 mmol) and 1-butanesulfonamide (405.7 mg, 2.96 mmol) in NMP (6 mL) at 25° C. was treated with cesium carbonate (1.93 g, 5.91 mmol) and stirred at 25° C. until complete (˜1 h, LCMS indicated complete adduct formation by displacement of fluoride). To the reaction mixture was then added allyl iodide (0.32 mL, 3.55 mmol). The reaction was heated to 150° C. for 30 min following addition of the allyl iodide. The reaction mixture was poured into 60 mL of water and extracted with 3×20 mL TBME. The combined organics were rinsed with 10 mL of brine, dried with MgSO₄, filtered, and concentrated to dryness. Purification was achieved by chromatography on silica using 10-55% EtOAc/hexanes. MS=308.9 (M+1).

### Step 2. 1-allyl-3-propyl-1,9-dihydro-[1,2]thiazino[4,3-g]indole 2,2-dioxide

1-allyl-8-nitro-3-propyl-1H-benzo[c][1,2]thiazine 2,2-dioxide was converted to 1-allyl-3-propyl-1,9-dihydro-[1,2]thiazino[4,3-g]indole 2,2-dioxide following the procedure outlined in step 5 of Preparatory Example 2. MS=303.0 (M+1).

### Step 3. 3-propyl-1,9-dihydro-[1,2]thiazino[4,3-g]indole 2,2-dioxide

1-allyl-3-propyl-1,9-dihydro-[1,2]thiazino[4,3-g]indole 2,2-dioxide was converted to 3-propyl-1,9-dihydro-[1,2]thiazino[4,3-g]indole 2,2-dioxide using Deprotection Method A from Examples 1 and 2 (compounds 12 and 42). Purification was achieved by chromatography on silica using 10-40% EtOAc/hexanes. MS=262.9 (M+1).

### Step 4. 7-chloro-3-propyl-1,9-dihydro-[1,2]thiazino[4,3-g]indole 2,2-dioxide

3-propyl-1,9-dihydro-[1,2]thiazino[4,3-g]indole 2,2-dioxide was converted to 7-chloro-3-propyl-1,9-dihydro-[1,2]thiazino[4,3-g]indole 2,2-dioxide by following step 5 of Example 14 (compound 22). Purification was achieved by chromatography on silica using 20-60% EtOAc/hexanes. ¹H NMR (400 MHz, Acetone-d₆) δ 10.59 (s, 1H), 7.57 (d, J=2.1 Hz, 1H), 7.36 (d, J=8.3 Hz, 1H), 7.30-7.23 (m, 2H), 2.64 (t, J=7.5 Hz, 2H), 1.78 (h, J=7.4 Hz, 2H), 1.03 (t. J=7.3 Hz, 3H). MS=296.9 (M+1).

### Example 16

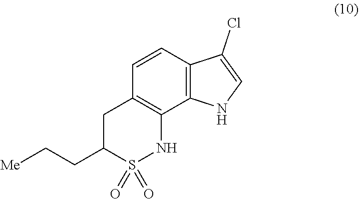 [SMILES: CCCC1Cc2ccc3c(Cl)c[nH]c3c2NS1(=O)=O]

### 7-chloro-3-propyl-1,3,4,9-tetrahydro-[1,2]thiazino[4,3-g]indole 2,2-dioxide (Compound 10)

Compound 10 was prepared in an analogous manner to that outlined in Scheme 13.

### Step 1. 3-propyl-1,3,4,9-tetrahydro-[1,2]thiazino[4,3-g]indole 2,2-dioxide

The starting material 3-propyl-1,9-dihydro-[1,2]thiazino[4,3-g]indole 2,2-dioxide was prepared as described in Example 15 (compound 9), step 3. A solution of 3-propyl-1,9-dihydro-[1,2]thiazino[4,3-g]indole 2,2-dioxide (9.0 mg, 0.034 mmol) and palladium (5.48 mg, 0.005 mmol, 10 wt. % on C) in methanol (1 mL) was sparged with nitrogen for 3 mins. The reaction mixture was then carefully sparged with a hydrogen balloon for 1 minute and then capped under the balloon. After 1 h, LCMS indicates complete hydrogenation. The reaction mixture was carefully sparged with nitrogen for 3 min and the reaction mixture filtered through a pad of celite. Volatiles were removed by concentration under reduced pressure. The product was used without further purification. MS=265.0 (M+1).

### Step 2. 7-chloro-3-propyl-1,3,4,9-tetrahydro-[1,2]thiazino[4,3-g]indole 2,2-dioxide (10)

3-propyl-1,3,4,9-tetrahydro-[1,2]thiazino[4,3-g]indole 2,2-dioxide was converted to 7-chloro-3-propyl-1,3,4,9-tetrahydro-[1,2]thiazino[4,3-g]indole 2,2-dioxide (10) by following step of Example 14. Purification was achieved by chromatography on silica using 20-60% EtOAc/hexanes. ¹H NMR (400 MHz, Acetone-d₆) δ 10.29 (s, 1H), 7.40 (s, 1H), 7.27 (d, J=8.2 Hz, 1H), 7.00 (d, J=8.2 Hz, 1H), 3.61 (dd, J=16.1, 4.4 Hz, 1H), 3.41-3.23 (m, 2H), 2.04-1.93 (m, 1H), 1.71-1.51 (m, 3H), 0.97 (t, J=7.2 Hz, 3H). MS=298.9 (M+1).

### Examples 17 and 18

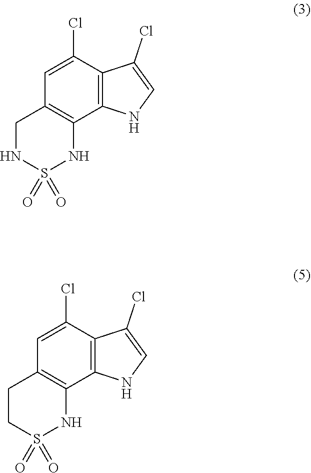 [SMILES: O=S1(=O)NCc2cc(Cl)c3c(Cl)c[nH]c3c2N1 ; O=S1(=O)CCc2cc(Cl)c3c(Cl)c[nH]c3c2N1]

### 6,7-dichloro-1,3,4,9-tetrahydro-[1,2,6]thiadiazino[4,3-g]indole 2,2-dioxide (Compound 3) and 6,7-dichloro-1,3,4,9-tetrahydro-[1,2]thiazino[4,3-g]indole 2,2-dioxide (Compound 5)

Compounds 3 and 5 were prepared in an analogous manner to that outlined in Scheme 14.

### Step 1. 6,7-dichloro-1,3,4,9-tetrahydro-[1,2,6]thiadiazino[4,3-g]indole 2,2-dioxide (3)

1-allyl-6,7-dichloro-1,3,4,9-tetrahydro-[1,2,6]thiadiazino[4,3-g]indole 2,2-dioxide was converted to 6,7-dichloro-1,3,4,9-tetrahydro-[1,2,6]thiadiazino[4,3-g]indole 2,2-dioxide using Deprotection Method A from Examples 1 and 2 (compounds 12 and 42). Purification was achieved by chromatography on silica using 5-15% 3:1 EtOAc:EtOH/CH₂Cl₂. ¹H NMR (400 MHz, Acetone-d₆) δ 10.48 (s, 1H), 8.66 (s, 1H), 7.49 (s, 1H), 6.97 (s, 1H), 6.36 (s, 1H), 4.73 (d, J 5.9 Hz, 2H). MS=289.8 (M−1).

### Step 1. 6,7-dichloro-1,3,4,9-tetrahydro-[1,2]thiazino[4,3-g]indole 2,2-dioxide (5)

1-allyl-6,7-dichloro-1,3,4,9-tetrahydro-[1,2]thiazino[4,3-g]indole 2,2-dioxide was converted to 6,7-dichloro-1,3,4,9-tetrahydro-[1,2]thiazino[4,3-g]indole 2,2-dioxide using Deprotection Method A from Examples 1 and 2 (compounds 12 and 4. Purification was achieved by chromatography on silica using 5-15% 3:1 EtOAc:EtOH/CH₂Cl₂. ¹H NMR (400 MHz, Acetone-d₆) δ 10.60 (s, 1H), 8.36 (s, 1H), 7.48 (d, J=2.5 Hz, 1H), 7.01 (s, 1H), 3.57-3.48 (m, 2H), 3.48-3.38 (m, 2H). MS=288.8 (M−1).

### Examples 80 and 82

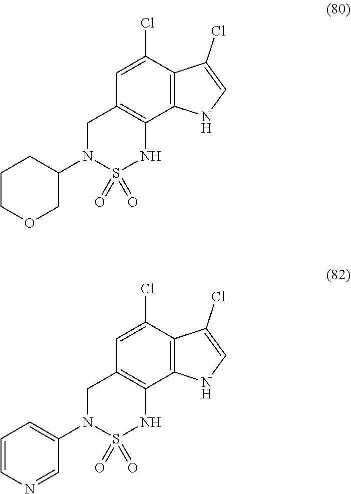 [SMILES: O=S1(=O)Nc2c(cc(Cl)c3c(Cl)c[nH]c23)CN1C1CCCOC1 ; O=S1(=O)Nc2c(cc(Cl)c3c(Cl)c[nH]c23)CN1c1cccnc1]

### 7-chloro-3-(tetrahydro-2H-pyran-3-yl)-1,3,4,9-tetrahydro-[1,2,6]thiadiazino[4,3-g]indole 2,2-dioxide (Compound 80) and 7-chloro-3-(pyridin-3-yl)-1,3,4,9-tetrahydro-[1,2,6]thiadiazino[4,3-g]indole 2,2-dioxide (Compound 82)

Compounds 80 and 82 were prepared in an analogous manner to that outlined in Scheme 15.

### Step 1. 2-(2-fluoro-3-nitrophenyl)-1,3-dioxolane

A solution of ethylene glycol (1.38 mL, 24.7 mmol) and 2-fluoro-3-nitro-benzaldehyde (417 mg, 2.47 mmol) in Toluene (16.4 mL) was treated with p-toluenesulfonic acid monohydrate (140.7 mg, 0.74 mmol). The resulting mixture was equipped with a Dean Stark trap and Vigreux Column. The reaction mixture was heated to 135° C. for 3 h. Upon cooling, the reaction mixture was poured into 30 mL of saturated aqueous NaHCO₃ and extracted with 3×20 mL EtOAc. The combined organics were rinsed with 10 mL of brine, dried with MgSO₄, filtered, and concentrated to dryness. The product was used without further purification. MS=214.0 (M+1).

### Step 2. N-allyl-N-(dimethylsulfamoyl)-2-(1,3-dioxolan-2-yl)-6-nitro-aniline

A solution of 2-(2-fluoro-3-nitrophenyl)-1,3-dioxolane (403 mg, 1.89 mmol) and 3-(dimethylsulfamoylamino)prop-1-ene (341.5 mg, 2.08 mmol) in DMF (6 mL) at room temperature was treated with cesium carbonate (739.2 mg, 2.27 mmol) and stirred at 100° C. until complete by LCMS (˜1 h). The reaction mixture was poured into 60 mL of water and extracted with 3×20 mL TBME. The combined organics were rinsed with 10 mL of brine, dried with MgSO₄, filtered, and concentrated to dryness. Purification was achieved by chromatography on silica using 15-45% EtOAc/hexanes. MS=357.9 (M+1).

### Step 3. N-allyl-N-(dimethylsulfamoyl)-6-(1,3-dioxolan-2-yl)-1H-indol-7-amine

N-allyl-N-(dimethylsulfamoyl)-2-(1,3-dioxolan-2-yl)-6-nitro-aniline was converted to N-allyl-N-(dimethylsulfamoyl)-6-(1,3-dioxolan-2-yl)-1H-indol-7-amine following the procedure outlined in step 5 of Preparatory Example 2. Purification was achieved by chromatography on silica using 10-40% EtOAc/hexanes. MS=351.9 (M+1).

### Step 4. 7-[allyl(dimethylsulfamoyl)amino]-3-chloro-1H-indole-6-carbaldehyde

A solution of 1-allyl-6-chloro-1,3,4,9-tetrahydro-[1,2]thiazino[4,3-g]indole 2,2-dioxide (154 mg, 0.52 mmol) in DMF (2.0 mL) was treated with NCS (69.2 mg, 0.52 mmol) and stirred at 90° C. for 45 min. The reaction mixture was cooled to room temperature and treated with 1 mL of 1 M aqueous HCl. The resulting mixture stirred for 1 h. The reaction mixture was poured into 30 mL of water and extracted with 3×15 mL TBME. The combined organics were rinsed with 10 mL of brine, dried with MgSO₄, filtered, and concentrated to dryness. Purification was achieved by chromatography on silica using 10-30% EtOAc/hexanes. MS=341.9 (M+1).

### Step 5. N-allyl-3-chloro-N-(dimethylsulfamoyl)-6-[(tetrahydropyran-3-ylamino)methyl]-1H-indol-7-amine

A mixture of 7-[allyl(dimethylsulfamoyl)amino]-3-chloro-1H-indole-6-carbaldehyde (10.5 mg, 0.031 mmol), tetrahydropyran-3-ylamine (4.7 mg, 0.046 mmol, 4.8 μL) and 3 Å molecular sieves (12.5 mg) in methanol (0.75 mL) was heated at 63° C. for 3 h. The mixture was cooled to 0° C. and sodium borohydride (1.7 mg, 0.046 mmol) was added. The ice bath removed and the mixture was allowed to warm to room temperature for 1 h. The reaction mixture was filtered and concentrated to dryness. The reaction mixture was poured into 20 mL of water and extracted with 3×15 mL EtOAc. The combined organics were rinsed with 10 mL of brine, dried with MgSO₄, filtered, and concentrated to dryness. The product was used without further purification. MS=426.9 (M+1).

### Step 6. 7-chloro-3-(tetrahydro-2H-pyran-3-yl)-1,3,4,9-tetrahydro-[1,2,6]thiadiazino[4,3-g]indole 2,2-dioxide

To make the compound 80, the Deprotection Method B from Examples 1 and 2 (compounds 12 and 42) was used. Following the standard deprotection at 70° C. for 2 h, the reaction mixture was heated to 83° C. for an additional 1.5 h. This was sufficient to drive cyclization to the sulfamide following deallylation. Purification was achieved by chromatography on silica using 15-45% EtOAc/hexanes. ¹H NMR (400 MHz, Acetone-d₆) δ 10.29 (s, 1H), 8.82 (s, 1H), 7.42 (s, 1H), 7.28 (d, J=8.2 Hz, 1H), 6.98 (d, J=8.2 Hz, 1H), 4.99-4.84 (m, 2H), 3.83 (ddd, J=10.8, 4.2, 1.9 Hz, 1H), 3.78-3.65 (m, 2H), 3.40-3.30 (m, 1H), 3.25 (td, J=11.3, 2.7 Hz, 1H), 1.90-1.80 (m, 1H), 1.80-1.68 (m, 1H), 1.68-1.59 (m, 1H), 1.59-1.47 (m, 1H). MS=339.9 (M−1).

### Step 5. N-allyl-3-chloro-N-(dimethylsulfamoyl)-6-[(3-pyridylamino)methyl]-1H-indol-7-amine

An oven dried vial was charged with 3-pyridinamine (5.6 mg, 0.060 mmol), 7-[allyl(dimethylsulfamoyl)amino]-3-chloro-1H-indole-6-carbaldehyde (22.4 mg, 0.065 mmol), and EtOAc (0.4 mL). TFA (9.1 μL, 0.119 mmol) was then added and the reaction stirred at 25° C. for 10 min. Sodium triacetoxy borohydride (16.4 mg, 0.077 mmol) was added and the reaction left to stir for 10 min. The reaction mixture was quenched by the addition of 180 μL of 1 N NaOH. The reaction mixture was poured into 10 mL of water and extracted with 3×10 mL EtOAc. The combined organics were rinsed with 10 mL of brine, dried with MgSO₄, filtered, and concentrated to dryness. Purification was achieved by chromatography on silica using 20-40% 3:1 EtOAc:EtOH/hexanes. MS=419.9 (M+1).

### Step 6. 7-chloro-3-(tetrahydro-2H-pyran-3-yl)-1,3,4,9-tetrahydro-[1,2,6]thiadiazino[4,3-g]indole 2,2-dioxide

To make the compound 82, Deprotection Method B from Examples 1 and 2 (compounds 12 and 42) was used. Following the standard deprotection at 70° C. for 2 h, the reaction mixture was heated to 83° C. for an additional 1.5 h. This was sufficient to drive cyclization to the sulfamide following deallylation. Purification was achieved by chromatography on silica using 30-80% EtOAc/hexanes. ¹H NMR (400 MHz, Acetone-d₆) δ 10.44 (s, 1H), 8.48 (d, J=2.5 Hz, 1H), 8.42 (d, J=5.0 Hz, 1H), 7.56 (d, J=7.8 Hz, 1H), 7.46 (d, J=2.3 Hz, 1H), 7.42 (d, J=8.2 Hz, 1H), 7.31 (dd, J=8.5, 4.7 Hz, 1H), 7.14 (d, J=8.3 Hz, 1H), 5.33 (s, 2H). MS=334.9 (M+1).

TABLE 8
The following compounds were prepared using procedures similar to those
described for Examples 10 and 11 (compounds 39 and 45) using the appropriate starting
materials.

| | | | Exact Mass |
|---|---|---|---|
| Compd. | | | [M + H]⁺ or |
| No. | Structure | Name | [M − H]⁻ |
| 4 | 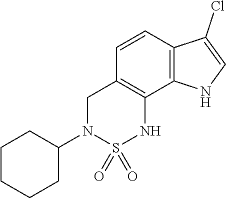 [SMILES: O=S1(=O)Nc2c(ccc3c(Cl)c[nH]c23)CN1C1CCCCC1] | 7-chloro-3-cyclohexyl- 1,3,4,9-tetrahydro-[1,2,6] thiadiazino[4,3-g]indole 2,2- dioxide | Calc'd 373.04, found 373.8 |
| 7 | 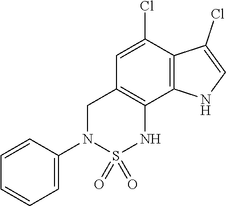 [SMILES: O=S1(=O)Nc2c(cc(Cl)c3c(Cl)c[nH]c23)CN1c1ccccc1] | 6,7-dichloro-3-phenyl- 1,3,4,9-tetrahydro-[1,2,6] thiadiazino[4,3-g]indole 2,2- dioxide | Calc'd 366.99, found 365.8 |

### Example 19

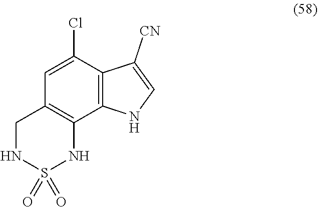 [SMILES: N#Cc1c[nH]c2c3c(cc(Cl)c12)CNS(=O)(=O)N3]

### 6-chloro-1,3,4,9-tetrahydro-[1,2,6]thiadiazino[4,3-g]indole-7-carbonitrile 2,2-dioxide (Compound 58)

Compound 58 was prepared in an analogous manner to that outlined in Scheme 16.

### Step 1. 1-allyl-6-chloro-1,3,4,9-tetrahydro-[1,2,6]thiadiazino[4,3-g]indole-7-carbaldehyde 2,2-dioxide

The starting material 1-allyl-6-chloro-1,3,4,9-tetrahydro-[1,2,6]thiadiazino[4,3-g]indole 2,2-dioxide was prepared using the procedure outlined in step 5 of Preparatory Example 2. To a stirred solution of phosphorus oxychloride (401.7 mg, 2.62 mmol) was added DMF (2 ml) dropwise at 0° C. and the mixture was stirred at this temperature for 20 mins. After this time, 1-allyl-6-chloro-1,3,4,9-tetrahydro-[1,2,6]thiadiazino[4,3-g]indole 2,2-dioxide (130 mg, 0.44 mmol) dissolved in DMF (2 mL) was added slowly. The mixture was stirred for 1 h and then the reaction mixture was poured into ice-water and quenched with aqueous saturated NaHCO₃ solution. The mixture was extracted with EtOAc (3×10 ml) and the combined organic layer was washed with brine (10 mL), dried over Na₂SO₄ and concentrated to dryness. MS=324.0 (M−1).

### Step 2. 1-allyl-6-chloro-1,3,4,9-tetrahydro-[1,2,6]thiadiazino[4,3-g]indole-7-carbaldehyde 2,2-dioxide oxime

To a stirred solution of 1-allyl-6-chloro-1,3,4,9-tetrahydro-[1,2,6]thiadiazino[4,3-g]indole-7-carbaldehyde 2,2-dioxide (142 mg, 0.40 mmol) in EtOH (10 ml) were added sodium acetate trihydrate (65.8 mg, 0.80 mmol) and hydroxylamine hydrochloride (41.8 mg, 0.60 mmol) at 0° C. Then the mixture was warmed to room temperature and stirred at this temperature for 2 h. The solvent was removed and the residue was dissolved in EtOAc (20 ml), then washed with water (20 mL) and brine (10 mL). The organic phase was dried over Na₂SO₄, concentrated to dryness and purified by Prep-TLC (EtOAc/PE=1:1) to give 1-allyl-6-chloro-1,3,4,9-tetrahydro-[1,2,6]thiadiazino[4,3-g]indole-7-carbaldehyde 2,2-dioxide oxime.

### Step 3. 1-allyl-6-chloro-1,3,4,9-tetrahydro-[1,2,6]thiadiazino[4,3-g]indole-7-carbonitrile 2,2-dioxide

To a solution of 1-allyl-6-chloro-1,3,4,9-tetrahydro-[1,2,6]thiadiazino[4,3-g]indole-7-carbaldehyde 2,2-dioxide oxime (90 mg, 0.26 mmol) in THF (5 ml) was added di(1H-imidazol-1-yl)methanone (94.1 mg, 0.53 mmol) at 0° C. The mixture was at this temperature for 1 h. The mixture was poured into water (20 ml) and extracted with EtOAc (3×10 ml). The combined organic was washed with 1N HCl (10 mL) and brine (10 mL), dried over Na₂SO₄, concentrated to dryness and purified by Prep-TLC (EtOAc/PE=1:1) to afford 1-allyl-6-chloro-1,3,4,9-tetrahydro-[1,2,6]thiadiazino[4,3-g]indole-7-carbonitrile 2,2-dioxide. MS=321.0 (M−1).

### Step 4. 6-chioro-1,3,4,9-tetrahydro-[1,2,6]thiadiazino[4,3-g]indole-7-carbonitrile 2,2-dioxide

1-allyl-6-chloro-1,3,4,9-tetrahydro-[1,2,6]thiadiazino[4,3-g]indole-7-carbonitrile 2,2-dioxide was converted to 6-chloro-1,3,4,9-tetrahydro-[11,2,6]thiadiazino[4,3-g]indole-7-carbonitrile 2,2-dioxide (58) using Deprotection Method A from Examples 1 and 2 (compounds 12 and 42). Purification was achieved by Prep-TLC (EtOAc/PE=2:1). ¹H NMR (400 MHz, CD₃OD): δ 8.02 (s, 1H), 7.04 (s, 1H), 4.54 (s, 2H). MS=281.0 (M−1).

TABLE 9
The following compounds were prepared using procedures similar to those
described for Example 19, compound 58 using the appropriate starting materials.

| | | | Exact Mass |
|---|---|---|---|
| Compd. | | | [M + H]⁺ or |
| No. | Structure | IUPAC Name | [M − H]⁻ |
| 8 | 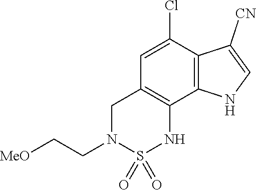 [SMILES: COCCN1Cc2cc(Cl)c3c(C#N)c[nH]c3c2NS1(=O)=O] | 6-chloro-3-(2-methoxyethyl)- 1,3,4,9-tetrahydro-[1,2,6] thiadiazino[4,3-g]indole7- carbonitrile 2,2-dioxide | Calc'd 341.04, found 340.8 |
| 99 | 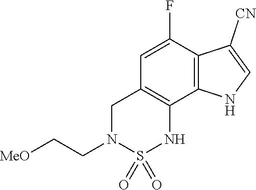 [SMILES: COCCN1Cc2cc(F)c3c(C#N)c[nH]c3c2NS1(=O)=O] | 6-fluoro-3-(2-methoxyethyl)- 1,3,4,9-tetrahydro-[1,2,6] thiadiazino[4,3-g]indole-7- carbonitrile 2,2-dioxide | Calc'd 323.07, found 323.0 |
| 100 | 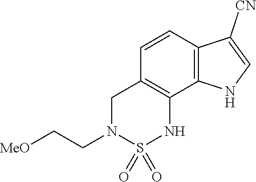 [SMILES: COCCN1Cc2ccc3c(C#N)c[nH]c3c2NS1(=O)=O] | 3-(2-methoxyethyl)- 1,3,4,9-tetrahydro-[1,2,6] thiadiazino[4,3-g]indole-7- carbonitrile 2,2-dioxide | Calc'd 307.08, found 307.0 |

### Example 20

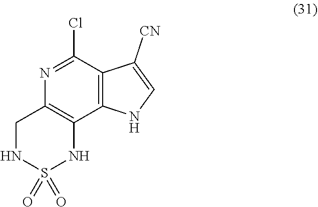 [SMILES: N#Cc1c[nH]c2c3c(nc(Cl)c12)CNS(=O)(=O)N3]

### 6,7-dichloro-1,3,4,9-tetrahydropyrrolo[2′,3′:4,5]pyrido[3,2-c][1,2,6]thiadiazine 2,2-dioxide (Compound 31)

Compound 31 was prepared in an analogous manner to that outlined in Scheme 17.

### Step 1. 2-(aminomethyl)-6-chloropyridin-3-amine

The procedure from Preparatory Example 1, step 1 was used with the following modifications. The reaction ran for 1 h at 0° C. and was then left to stir at room temperature for 4 h. The reaction mixture was quenched by the careful addition of EtOH. Once effervescence ceased, the reaction was stirred a further 20 min during which time a precipitate formed. The reaction was then carefully acidified with 4 N HCl in dioxane while stirring. This induced formation of a yellow precipitate from what was an orange solution prior to acid addition. The precipitate was filtered and rinsed with TBME. The filtered product was suspended in saturated aqueous ammonia solution (60 mL) and extracted with 3×60 mL of 30% IPA/CHCl₃. The combined organics were rinsed with 20 mL of brine, dried with MgSO₄, filtered, and concentrated to dryness. The resulting product was used without further purification. MS=158.0 (M+1).

### Step 2. 6-chloro-3,4-dihydro-1H-pyrido[3,2-c][1,2,6]thiadiazine 2,2-dioxide

The procedure from Preparatory Example 1, step 2 was used with the following modifications. The reaction was heated for 9 h. Purification was achieved by chromatography on silica using 5-30% 3:1 EtOAc:EtOH/hexanes. MS=219.9 (M+1).

### Step 3. 6-chloro-8-nitro-3,4-dihydro-1H-pyrido[3,2-c][1,2,6]thiadiazine 2,2-dioxide

To a solution of 6-chloro-3,4-dihydro-1H-pyrido[3,2-c][1,2,6]thiadiazine 2,2-dioxide (486 mg, 2.21 mmol) and sodium nitrite (183.2 mg, 2.66 mmol) in acetonitrile (11 mL) was added PIFA (1.14 g, 2.66 mmol) at room temperature. After 3 h, the reaction was quenched by the addition 30 mL water. The aqueous layer was extracted with ethyl acetate (3×15 mL). The combined organic layers were washed with brine (10 mL), dried over anhydrous MgSO₄, filtered and concentrated. The residue was used without further purification. MS=262.8 (M−1).

### Step 4. 1-allyl-6-chloro-8-nitro-3,4-dihydro-1H-pyrido[3,2-c][1,2,6]thiadiazine 2,2-dioxide

A mixture of the unpurified 6-chloro-8-nitro-3,4-dihydro-1H-pyrido[3,2-c][1,2,6]thiadiazine 2,2-dioxide (30.5 mg, 0.12 mmol) and cesium carbonate (22.5 mg, 0.069 mmol) was dissolved in DMF (0.5 mL) and stirred at 100° C. for 10 min. After cooling to room temperature, the reaction mixture was treated with allyl iodide (12.6 μL, 0.14 mmol) and stirred at 100° C. for 20 min. The reaction mixture was poured into 30 mL of water and extracted with 3×15 mL TBME. The combined organics were rinsed with 10 mL of brine, dried with MgSO₄, filtered, and concentrated to dryness. Purification was achieved by chromatography on silica using 10-30% EtOAc/hexanes. MS=302.8 (M−1).

### Step 5. 1-allyl-6-chloro-1,3,4,9-tetrahydropyrrolo[2′,3′:4,5]pyrido[3,2-c][1,2,6]thiadiazine 2,2-dioxide

The procedure from Preparatory Example 2, step 5 was used with the following modifications. Purification was achieved by chromatography on silica using 20-70% EtOAc/hexanes. The main product is a partially reduced derivative of the desired product (M+3) which can slowly oxidize to the desired product 1-allyl-6-chloro-1,3,4,9-tetrahydropyrrolo[2′,3′:4,5]pyrido[3,2-c][1,2,6]thiadiazine 2,2-dioxide on standing in air. This process can be hastened by dissolving the partially reduced derivative in acetone and heating the resulting mixture to 40° C. with vigorous stirring under air. The 1-allyl-6-chloro-1,3,4,9-tetrahydropyrrolo[2′,3′:4,5]pyrido[3,2-c][1,2,6]thiadiazine 2,2-dioxide delivered by air oxidation in acetone can be used without further purification simply by concentration and removal of the acetone under vacuum. MS=298.9 (M+1).

### Step 6. 1-allyl-6,7-dichloro-1,3,4,9-tetrahydropyrrolo[2′,3′:4,5]pyrido[3,2-c][1,2,6]thiadiazine 2,2-dioxide

The procedure from Example 14, compound 22, step 5 was used. Purification was achieved by chromatography on silica using 10-40% EtOAc/hexanes. MS=332.8 (M+1).

### Step 7. 6,7-dichloro-1,3,4,9-tetrahydropyrrolo[2′,3′:4,5]pyrido[3,2-c][1,2,6]thiadiazine 2,2-dioxide (31)

To make the compound 31, the Deprotection Method A from Examples 1 and 2 (compounds 12 and 42) was used. Purification was achieved by chromatography on reverse phase by injection of a MeOH solution of the product residue. 10-80% CH₃CN/Water was used as eluent. ¹H NMR (400 MHz, Acetone-d₆) δ 7.58 (s, 1H), 4.65 (s, 2H). MS=292.8 (M+1).

### Example 21

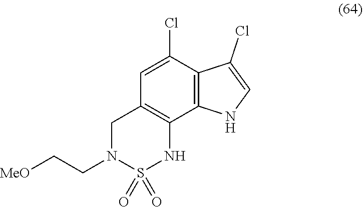 [SMILES: COCCN1Cc2cc(Cl)c3c(Cl)c[nH]c3c2NS1(=O)=O]

### 6,7-dichloro-3-(2-methoxyethyl)-1,3,4,9-tetrahydro-[1,2,6]thiadiazino[4,3-g]indazole 2,2-dioxide (Compound 64)

Compound 64 was prepared in an analogous manner to that outlined in Scheme 18.

### Step 1. (E)-2-(5-chloro-2-nitrophenyl)-N,N-dimethylethen-1-amine

To a solution of 1-chloro-2,5-dimethyl-4-nitrobenzene (5.0 g, 26.94 mmol) in DMF (40 mL) was added DMF-DMA (21.5 mL, 161.6 mmol) under Ar. The vessel was sealed and heated to 140° C. After 8 h, the solvent was removed under vacuum and the residue used without further purification.

### Step 2. 5-chloro-4-methyl-2-nitrobenzaldehyde

To a solution of NaIO₄ (17.3 g, 80.82 mmol) in a mixture of DMF (30 mL) and H₂O (60 mL) was added dropwise the unpurified (E)-2-(5-chloro-2-nitrophenyl)-N,N-dimethylethen-1-amine. The resulting mixture was stirred at 25° C. After 4 hrs, the reaction mixture was filtered through celite, the residue was partitioned between MTBE (250 mL) and H₂O (250 mL), separated, the organic layer was dried over Na₂SO₄, filtered, concentrated and purified by column chromatography on silica (PE:EA=20:1) to give 5-chloro-4-methyl-2-nitrobenzaldehyde. ¹H NMR (400 MHz, CDCl₃): δ 10.56-10.26 (br s, 1H), 7.92 (s, 1H), 8.02 (s, 1H), 2.59 (s, 3H).

### Step 3. N-(5-chloro-4-methyl-2-nitrobenzyl)-2-methoxyethan-1-amine

To a solution of 5-chloro-4-methyl-2-nitrobenzaldehyde (1.0 g, 5.0 mmol) in methanol (20 mL) was added 2-methoxyethylamine (752.6 mg, 10.02 mmol) at 0° C. After 0.5 h, the mixture was allowed to warm to 25° C. Then, after 3 hrs, the mixture was cooled back to 0° C. and NaBH₄ (228.5 mg, 6.01 mmol) was added. Then the ice bath was removed and the reaction allowed to warm back to 25° C. After 1 h, the mixture was partitioned between EtOAc (50 mL) and H₂O (50 mL), the organic layer was separated, dried over Na₂SO₄, filtered, concentrated and purified by column chromatography on silica (PE:EA=5:1-3:1) to give N-(5-chloro-4-methyl-2-nitrobenzyl)-2-methoxyethan-1-amine. ¹H NMR (400 MHz, CD₃OD): δ 7.98 (s, 1H), 7.69 (s, 1H), 3.99 (s, 2H), 3.53-3.43 (m, 2H), 3.33 (s, 3H), 2.75-2.76 (m, 2H), 2.44 (s, 3H).

### Step 4. 4-chloro-2-(((2-methoxyethyl)amino)methyl)-5-methylaniline

To a solution of N-(5-chloro-4-methyl-2-nitrobenzyl)-2-methoxyethan-1-amine (5.62 g, 21.72 mmol) in a mixture of absolute ethanol (60 mL) and water (20 mL) was added iron (6.08 g, 108.6 mmol) and NH₄Cl (11.6 g, 217.2 mmol). The resulting mixture was heated to 80° C. for 3 h. Afterwards, the mixture was filtered through celite and the filtrate concentrated to dryness (concentrated twice from toluene). The residue was purified by column chromatography on reverse phase (5% MeCN/H₂O to 50% MeCN/H₂O over 40 mins. MS=229.1 (M+1).

### Step 5. 6-chloro-3-(2-methoxyethyl)-7-methyl-3,4-dihydro-1H-benzo[c][1,2,6]thiadiazine 2,2-dioxide

To a solution of 4-chloro-2-(((2-methoxyethyl)amino)methyl)-5-methylaniline (3.1 g, 13.55 mmol) in pyridine (30 mL) was added sulfamide (3.9 g, 40.66 mmol). After the mixture was stirred at 130° C. for 8 h, the solvent was removed under vacuum, the residue was partitioned between H₂O (60 mL) and EtOAc (60 mL), the organic layer was separated, dried over Na₂SO₄, filtered, concentrated and purified by column chromatography on silica (PE:EA=3:1-1:1). MS=291.1 (M+1).

### Step 6. 6-chloro-3-(2-methoxyethyl)-7-methyl-8-nitro-3,4-dihydro-1H-benzo[c][1,2,6]thiadiazine 2,2-dioxide

To a solution of 6-chloro-3-(2-methoxyethyl)-7-methyl-3,4-dihydro-1H-benzo[c][1,2,6]thiadiazine 2,2-dioxide (250 mg, 0.86 mmol) in a mixture of CH₃CN (5 mL) and TFAA (1 mL) at 0° C. was added KNO₃ (95.5 mg, 0.95 mmol). After 1.5 h, the mixture was diluted with EtOAc (30 mL) and carefully adjusted to pH=6-7 with saturated aqueous NaHCO₃. The organic layer was separated, dried over Na₂SO₄, filtered, concentrated and purified by column chromatography on silica (PE:EA=3:1-1:1). ¹H NMR (400 MHz, CDCl₃) δ 7.32 (s, 1H), 4.87 (s, 2H), 3.73-3.51 (m, 2H), 3.33 (s, 3H), 3.32-3.15 (m, 2H), 2.42 (s, 3H).

### Step 7. 1-allyl-6-chloro-3-(2-methoxyethyl)-7-methyl-8-nitro-3,4-dihydro-1H-benzo[c][1,2,6]thiadiazine 2,2-dioxide

To a solution of 6-chloro-3-(2-methoxyethyl)-7-methyl-8-nitro-3,4-dihydro-1H-benzo[c][1,2,6]thiadiazine 2,2-dioxide (341.1 mg, 1.02 mmol) in DMF (8 mL) was added potassium 2-methylpropan-2-olate (171.7 mg, 1.53 mmol) at 0° C. The reaction mixture was allowed to warm to 25° C. and, after 20 mins, allyl iodide (204.78 mg, 1.22 mmol) was added. The reaction mixture was left to stir overnight (16 h), then partitioned between H₂O (80 mL) and EtOAc (40 mL), the organic layer was separated, dried over Na₂SO₄, filtered, concentrated and purified by Prep-TLC (PE:EA=2:1). MS=376.1 (M+1).

### Step 8. 1-allyl-8-amino-6-chloro-3-(2-methoxyethyl)-7-methyl-3,4-dihydro-1H-benzo[c][1,2,6]thiadiazine 2,2-dioxide

To a solution of 1-allyl-6-chloro-3-(2-methoxyethyl)-7-methyl-8-nitro-3,4-dihydro-1H-benzo[c][1,2,6]thiadiazine 2,2-dioxide (221.3 mg, 0.59 mmol) in a mixture of absolute ethanol (27 mL) and water (9 mL) was added iron (165.2 mg, 2.95 mmol) and NH₄Cl (314.8 mg, 5.9 mmol). The mixture was stirred at 75° C. for 2 h. The reaction mixture was filtered through celite, the residue was concentrated and partition between H₂O (40 mL) and EtOAc (40 mL), the organic layer was separated, dried over Na₂SO₄, filtered, and concentrated. The unpurified residue was used without further purification. MS=346.1 (M+1).

### Step 9. 1-allyl-6-chloro-3-(2-methoxyethyl)-1,3,4,9-tetrahydro-[1,2,6]thiadiazino[4,3-g]indazole 2,2-dioxide

To a solution of 1-allyl-8-amino-6-chloro-3-(2-methoxyethyl)-7-methyl-3,4-dihydro-1H-benzo[c][1,2,6]thiadiazine 2,2-dioxide (210.6 mg, 0.33 mmol) in acetic acid (6 mL) was added NaNO₂ (27.3 mg, 0.40 mmol). After 2 h, the solvent was removed in vacuum, the residue was partitioned between EtOAc (30 mL) and saturated aqueous NaHCO₃ solution (30 mL), the organic layer was separated, dried over Na₂SO₄, filtered, concentrated and purified by Prep-TLC (PE:EA=2:1). MS=357.0 (M+1).

### Step 10. 1-allyl-6,7-dichloro-3-(2-methoxyethyl)-1,3,4,9-tetrahydro-[1,2,6]thiadiazino[4,3-g]indazole 2,2-dioxide

To a solution of 1-allyl-6-chloro-3-(2-methoxyethyl)-1,3,4,9-tetrahydro-[1,2,6]thiadiazino[4,3-g]indazole 2,2-dioxide (55.6 mg, 0.18 mmol) in absolute ethanol (16.8 mL) was added NaClO (1.6 mL, 10%, aqueous). The mixture was stirred at 30° C. and, after 2 hrs, the mixture was partitioned between EtOAc (60 mL) and H₂O (60 mL), the organic layer was separated, dried over Na₂SO₄, filtered, concentered and purified by Prep-TLC (PE:EA=3:1).

### Step 11. 6,7-dichloro-3-(2-methoxyethyl)-1,3,4,9-tetrahydro-[1,2,6]thiadiazino[4,3-g]indazole 2,2-dioxide (21)

To a solution of 1-allyl-6,7-dichloro-3-(2-methoxyethyl)-1,3,4,9-tetrahydro-[1,2,6]thiadiazino[4,3-g]indazole 2,2-dioxide (25.5 mg, 0.07 mmol) in acetonitrile (5 mL) was added 1,3-dimethylbarbituric acid (101.8 mg, 0.65 mmol) and Pd(PPh₃)₄(4.0 mg, 0.0035 mmol). The mixture was stirred at 70° C. After 2 h, the solvent was removed under vacuum, the residue was partitioned between EtOAc (30 mL) and H₂O (30 mL), the organic layer was separated, dried over Na₂SO₄, filtered, concentrated and purified by Prep-HPLC. ¹H NMR (400 MHz, CD₃OD) δ 6.98 (s, 1H), 4.84 (s, 2H), 3.61 (t, J=5.2 Hz, 2H), 3.33 (s, 3H), 3.18 (t, J=5.2 Hz, 2H). MS=349.0 (M−1).

### Examples 22 and 23

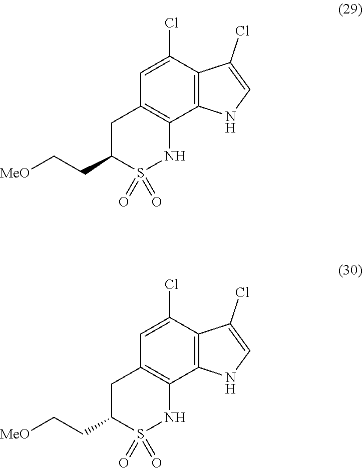 [SMILES: COCC[C@H]1Cc2cc(Cl)c3c(Cl)c[nH]c3c2NS1(=O)=O ; COCC[C@@H]1Cc2cc(Cl)c3c(Cl)c[nH]c3c2NS1(=O)=O]

### (R)-6,7-dichloro-3-(2-methoxyethyl)-1,3,4,9-tetrahydro-[1,2]thiazino[4,3-g]indole 2,2-dioxide (Compound 29) and (S)-6,7-dichloro-3-(2-methoxyethyl)-1,3,4,9-tetrahydro-[1,2]thiazino[4,3-g]indole 2,2-dioxide (Compound 30)

The racemic compound 6,7-dichloro-3-(2-methoxyethyl)-1,3,4,9-tetrahydro-[1,2]thiazino[4,3-g]indole 2,2-dioxide was resolved by chiral HPLC (Chiralpak AD-H; 45% ethanol in CO₂, 100 bar) to furnish the enantiopure compounds. The faster-eluting enantiomer of the title compound was obtained as a solid (Compound 29): NMR as above. ¹H NMR (400 MHz, CDCl₃) δ 8.83 (s, 1H), 7.14 (d, J=2.7 Hz, 1H), 7.10 (s, 1H), 6.88 (s, 1H), 3.68-3.50 (m, 4H), 3.35 (s, 4H), 2.39 (ddt, J=14.7, 7.4, 4.9 Hz, 1H), 1.85 (dtd, J=14.6, 6.9, 4.6 Hz, 1H). MS=346.8 (M−1). The slower-eluting enantiomer of the title compound was obtained as a solid (Compound 30): ¹H NMR (400 MHz, CDCl₃) δ 8.83 (s, 1H), 7.14 (d, J=2.7 Hz, 1H), 7.10 (s, 1H), 6.88 (s, 1H), 3.68-3.50 (m, 4H), 3.35 (s, 4H), 2.39 (ddt, J=14.7, 7.4, 4.9 Hz, 1H), 1.85 (dtd, J=14.6, 6.9, 4.6 Hz, 1H). MS=346.8 (M−1).

### Assays

The compounds of the present invention were subjected to various assays to determine their biological activity. A description of the assays used as well as assay results for particular compounds of the present invention are presented below.

Proteins used for the assays were as follows: Human DDA1 (UniProt:Q9BW61), DDB1 (UniPro: Q16531), and DCAF15 (UniProt: Q66K64) coding sequences were cloned into pFastBac1 vectors and were co-expressed in Sf9 cells using Bac-to-Bac baculovirus expression system (Thermo Fisher Scientific). The expression construct for DCAF15 includes a N-terminal His6-tag to facilitate the purification. The atrophin-1 homology region of DCAF15 (amino acid residues 276-383) was excised (Δpro, originally to facilitate crystallization). Triplex of DDA1-DDB1ΔB-DCAF15Δpro, which has the BPB domain (aa 396 to 705) excised and replaced with a GNGNSG linker, was used in all the kinetic measurements because of the higher yield. R1R2 of RBM39 (aa 150 to 331; UniProt: Q14498) contained a C-terminal FLAG tag used in the DCAF15/RBM39 complex formation assay.

### DCAF15 RBM39 Complex Formation Assay

LANCE Eu-W1024 Anti-6×His and SureLight Allophycocyanin-anti-FLAG antibodies were purchased from Perkin Elmer. Human DDB1DB and His-tagged DCAF15 were co-expressed in baculovirus/sf9 system. While full length DDB1 contains three 7-bladed propellers termed A, B, and C, DDB1DB has only propeller A (amino acids 1 to 395) and C (amino acids 709-1140). The His-tagged DCAF15 contains six histidine residues directly preceding the DCAF15 sequence without any linker. An RBM39 fragment containing RRM1 and RRM2 was expressed in E. coli. Amino acids 150 to 331 of RBM39 were cloned into pGEX4T3 with an N-terminal TEV cleavage site and a C-terminal FLAG tag.

A compound of the present disclosure in DMSO (2 μL) was transferred to a 384-well assay plate. Compound was added in 14 points, 3-fold serial dilution (1 μM to 0.6 pico molar) in DMSO. 48 mL FRET assay buffer (25 mM HEPES, pH 7.5, 100 mM NaCl, 0.1 M BSA, 0.5 mM TCEP, 0.005% Tween-20, 0.2 nM Eu-W1024 Anti-6×His, 50 nM SureLight Allophycocyanin-anti-FLAG, 20 nM DDB1DB/DCAF15, 50 nM RBM39 R1R2) was added and the resulting solution was incubated overnight at 4° C. The plate was placed in a plate reader (Spark 10) with excitation at l=340 nm and emissions monitored at l=665 nm and l=615 nm, and the FRET ratio (Em 665/Em 615) was determined. Low signal controls containing no compound and high signal controls containing 10 mM indisulam were measured for each plate.

EC₅₀ values were calculated using Dotmatics template equation for 4-parameter fit.

Results are expressed as % formation using the following equation:

$$\% formation = ( \frac{high control - sample}{high control - low control} ) \times 100$$

### DCAF15 Displacement Assay (Displacement FRET K_{D})

The previously described procedure was followed (See X. Du, et al., “Structural Basis and Kinetic Pathway of RBM39 Recruitment to DCAF15 by a Sulfonamide Molecular Glue E7820”, Structure, 27, 1625-1633.e3, 2019). Synthesis of the probe PT7795 is described in the article.

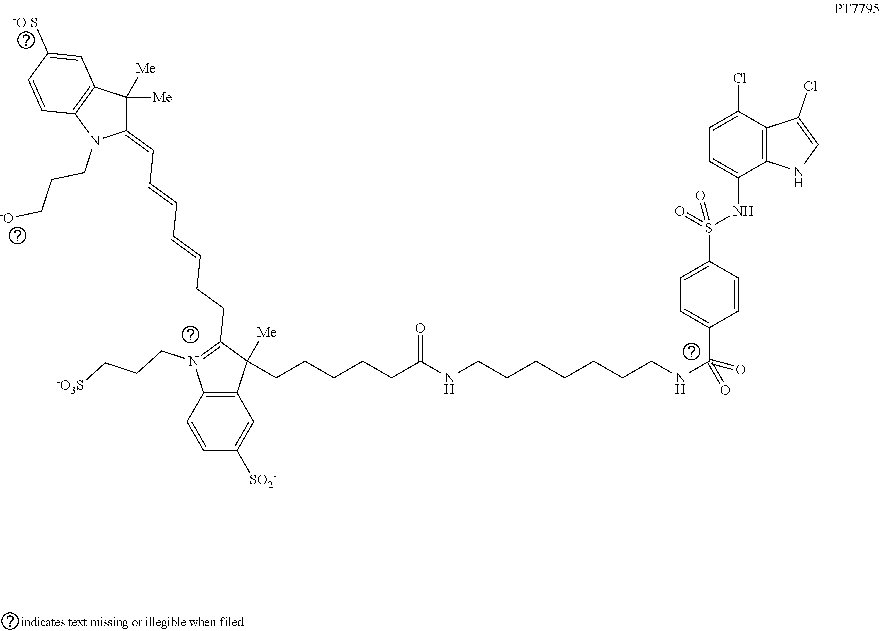

Binding of PT7795 to DDA1-DDB1AB-DCAF15Δpro was measured by the FRET signal between europium-labeled anti-His antibody (AD0205, Perkin Elmer) that is associated with His-tagged DCAF15 and ALX647 moiety in PT7795. The concentration of PT7795 was varied from 2.3 nM to 1000 nM in 16 wells, each containing 25 mM HEPES pH7.5, 100 mM NaCl, 0.1 mg/ml BSA, 0.005% Tween 20, 0.5 mM TCEP, 4% DMSO, 0.5 nM europium-labeled anti-His antibody, and 10 nM triplex of DDA1-DDB1DB-DCAF15 Δpro. After equilibrating for 2-3 hours at 4° C., the plate was removed from the incubator, briefly spun, then read on a Spark 10 (Tecan) at the excitation/emission wavelength of 340 nm/615 nm and 340 nm/665 nm. To obtain Kd, the ratio of 665/615 readings was fitted to Equation 1, where F stands for the ratio of 665/615 nM.

F=Fₘₐₓ[PT7795]/(K_{d}+[PT7795]) (Equation 1)

Binding of compounds in this disclosure to DCAF15 was measured in a competition by the displacement of PT7795. The concentration of PT7795 was kept at 300 nM while the concentration of a compound was varied from 0.01 to 100 μM. The incubation time was increased to 18 hours for enhanced signal to noise level. To obtain IC50, the ratio of 665/615 readings was fitted to Equation 2, where n is Hill slope.

F=Fₘᵢₙ+(Fₘₐₓ−Fₘᵢₙ)/(1+10^{(logIC50-log[Compound])}^{ⁿ}) (Equation 2)

The K_{d}s can then be calculated from IC₅₀s using Equation 3 (Cheng-Prusoff equation).

K_{d}=IC₅₀/(1+([PT7795]/K_{d}^{PT7795})) (Equation 3)

### CellTiter-Glo Assay

HCT116 human colorectal carcinoma cells were used in these experiments. HCT116 cells were obtained from ATCC. CellTiter-Glo® Luminescent Cell Viability Assay Reagent was obtained from Promega.

About 3,000 cells were seeded into 96-well plate with 100 μL of media (DMEM supplemented with 10% FBS, 100 units penicillin, and 100 mg streptomycin per mL) the day before the experiment. For compound treatment, the master plate was prepared as follows: 100× compound stocks were prepared in a 96-well plate by 1 to 3 serial dilutions of 0.5 mM DMSO stocks. Each master plate contained serial dilution of 8 compounds, including the control compound E7820. 1 mL of 100× compound stock was added to each well of the assay plate to give final concentrations of 5, 1.67, 0.56, 0.19, 0.06, 0.02, 0.0069, 0.002, 0.0007, 0.0002, and 0 mM. Each concentration was tested in triplicate. After 3 days, cell viability was determined using CellTiter-Glo Luminescent Cell Viability Assay Reagent following the manufacturer's recommended protocol. Briefly, 50 mL of CellTiter-Glo reagent was added to each well of the assay plate and the contents were mixed for 3 minutes at 600 rpm on a shaker (Thermomixer R). The plate was immediately placed in a plate reader (Synergy 2) and the luminescence signal (0.5 second per well integration time) was determined. IC₅₀ values were calculated using Dotmatics software.

Tables 10—shows biological activities of selected compounds described herein in the CellTiter-Glo assay, DCAF15 displacement assay and DCAF15 RBM39 ternary complex formation assay. Compound numbers correspond to the numbers and structures provided in Tables 1-9 and Examples 1-23.

TABLE 10

| | | DCAF15 | DCAF15 RBM39 |
|---|---|---|---|
| Compound | CTG | displacement | ternary complex |
| Number | IC₅₀ (μM) | IC₅₀ (μM) | EC₅₀ (μM) |
| 1 | 0.4753 | 3.1553 | 0.0055 |
| 2 | 2.2308 | 51.821 | 0.0658 |
| 3 | 1.4318 | 27.791 | 0.0081 |
| 4 | 0.9434 | 58.089 | 0.0161 |
| 5 | 0.7888 | 19.953 | 0.011 |
| 6 | 0.6084 | 5.2558 | 0.0031 |
| 7 | 1.3128 | 17.39 | 0.0088 |
| 8 | 3.1602 | 3.3379 | 0.0062 |
| 9 | 5 | 100 | 0.0747 |
| 10 | 5 | 100 | 0.0164 |
| 11 | 1.397 | 9.7161 | 0.0028 |
| 12 | 0.1253 | 1.1091 | 0.0007 |
| 13 | 0.8095 | 36.065 | 0.0062 |
| 14 | 0.2183 | 2.3206 | 0.0034 |
| 15 | 0.7256 | 14.369 | 0.0077 |
| 16 | 0.2588 | 1.2086 | 0.001 |
| 17 | 0.916 | 0.7969 | 0.0018 |
| 18 | 0.4175 | 2.6831 | 0.0009 |
| 19 | 0.2374 | 0.5309 | 0.0016 |
| 20 | 0.1548 | 1.0678 | 0.0007 |
| 21 | 0.4146 | 0.5605 | 0.0015 |
| 22 | 0.2338 | 12.28 | 0.0007 |
| 23 | 0.5913 | 9.5915 | 0.0028 |
| 24 | 0.7994 | 8.2995 | 0.0039 |
| 25 | 0.9916 | 52.219 | 0.0034 |
| 26 | 0.1301 | 0.5069 | 0.0012 |
| 27 | 0.1399 | 0.6468 | 0.0008 |
| 28 | 0.5012 | 28.005 | 0.0014 |
| 29 | 0.5547 | 50.549 | 0.0019 |
| 30 | 0.1836 | 2.5821 | 0.0013 |
| 31 | 4.5094 | 100 | 0.2633 |
| 32 | 0.2693 | 4.116 | 0.0019 |
| 33 | 0.1619 | 0.5298 | 0.0023 |
| 34 | 0.1257 | 0.6887 | 0.0018 |
| 35 | 0.1176 | 0.2103 | 0.004 |
| 36 | 0.2359 | 0.9634 | 0.0021 |
| 37 | 0.5679 | 1.999 | 0.0025 |
| 38 | 0.2625 | 0.4575 | 0.0013 |
| 39 | 4.9918 | 0.2851 | 0.0012 |
| 40 | 0.537 | 1.559 | 0.0016 |
| 41 | 0.1831 | 17.523 | 0.0106 |
| 42 | 0.2975 | 0.5881 | 0.0007 |
| 43 | 0.2465 | 1.0514 | 0.0027 |
| 44 | 0.0622 | 0.2761 | 0.0018 |
| 45 | 0.0528 | 0.1274 | 0.0016 |
| 46 | 0.2778 | 0.2936 | 0.0016 |
| 47 | 0.3932 | 0.3899 | 0.0004 |
| 48 | 0.2593 | 0.6233 | 0.0006 |
| 49 | 2.2541 | 8.3889 | 0.0007 |
| 50 | 0.881 | 5.7108 | 0.0011 |
| 51 | 0.2826 | 4.4114 | 0.0015 |
| 52 | 0.0937 | 0.5799 | 0.0006 |
| 53 | 0.3936 | 4.2778 | 0.0034 |
| 54 | 0.1275 | 0.371 | 0.0012 |
| 55 | 0.1082 | 0.1736 | 0.0017 |
| 56 | 0.1843 | 1.4357 | 0.0026 |
| 57 | 0.5247 | 1.0001 | 0.004 |
| 58 | 5 | 14.862 | 0.0462 |
| 59 | 0.1199 | 0.3249 | 0.002 |
| 60 | 2.8402 | 0.1623 | 0.0024 |
| 61 | N/A | 1.2423 | 0.1233 |
| 62 | N/A | 1.0656 | 0.1552 |
| 63 | 0.3513 | 0.3283 | 0.001 |
| 64 | 5 | 4.562 | 0.01 |
| 65 | 0.4928 | 0.9341 | 0.0019 |
| 66 | 5 | 100 | 0.1085 |
| 67 | 0.3199 | 0.4516 | 0.002 |
| 68 | 0.2047 | 0.6295 | 0.0023 |
| 69 | 1.2033 | 7.5615 | 0.0039 |
| 70 | 0.8333 | 1.95 | 0.0033 |
| 71 | 0.3526 | 0.9523 | 0.0021 |
| 72 | 0.15 | 0.2233 | 0.0012 |
| 73 | 0.4179 | 1.6498 | 0.0013 |
| 74 | 0.1708 | 0.3009 | 0.0013 |
| 75 | 0.8499 | 4.315 | 0.0023 |
| 76 | 0.2545 | 0.5687 | 0.0012 |
| 77 | N/A | 0.6799 | 0.0008 |
| 78 | N/A | 1.1693 | 0.0007 |
| 79 | N/A | 19.497 | 0.0089 |
| 80 | N/A | 1.8106 | 0.0182 |
| 81 | N/A | 32.013 | 0.0177 |
| 82 | N/A | 3.3187 | 0.0011 |
| 83 | 0.0669 | 0.7808 | 0.0023 |
| 84 | 1.791 | 33.051 | 0.0043 |
| 85 | 0.302 | 8.3799 | 0.002 |
| 86 | 0.1213 | 0.4102 | 0.0013 |
| 87 | 0.2585 | 6.2187 | 0.0018 |
| 88 | N/A | 0.2307 | 0.0016 |
| 89 | N/A | 0.574 | 0.0008 |
| 90 | N/A | 0.274 | 0.001 |
| 91 | N/A | 0.0518 | 0.0004 |
| 92 | N/A | 0.9648 | 0.0007 |
| 93 | N/A | 0.773 | 0.0016 |
| 94 | N/A | 0.3625 | 0.0029 |
| 95 | N/A | 0.0949 | 0.0012 |
| 96 | N/A | 0.2153 | 0.0012 |
| 97 | N/A | 0.0 | 0.0017 |
| 98 | N/A | 0.1811 | 0.0023 |
| 99 | N/A | 30.793 | 0.2121 |
| 100 | N/A | 100 | 0.1683 |

1. A compound of Formula I: 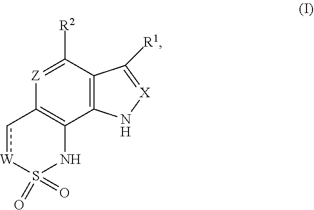
or a pharmaceutically acceptable salt thereof, wherein:
W is NR^{a} or CR^{a}R^{b};
R^{b} is hydrogen, or (C₁-C₄)alkyl;
R^{a} is selected from H, (C₁-C₆)alkyl, —(C₁-C₆)alkylOH, (C₁-C₆)alkoxy(C₁-C₆)alkyl), (C₁-C₆)haloalkyl, (C₂-C₈)alkenyl, cycloalkyl((C₀-C₆)alkyl), aryl((C₀-C₆)alkyl), heterocycloalkyl((C₀-C₆)alkyl), heteroaryl((C₀-C₆)alkyl), ((C₀-C₆)alkyl)amino((C₁-C₆)alkyl), and ((C₁-C₆)alkyl)carbonylamino((C₁-C₆)alkyl), wherein R^{a} is substituted with 0, 1, 2, or 2 R⁴ substituents;
each R⁴ independently is selected from (C₁-C₆)alkylcarbonyl, cyano, (C₁-C₆)haloalkyl, halogen, oxo, (C₁-C₆)alkyl, (C₁-C₆)alkoxy((C₀-C₆)alkyl), —SO₂(C₁-C₆)alkyl, amino, hydroxy, amino(C₁-C₆)akylcarbonyl, (C₁-C₆)alkylcarboxy, and —(C₁-C₆)alkylOH, provided that when R^{a} is (C₁-C₆)alkoxy(C₁-C₆)alkyl), R⁴ is other than (C₁-C₆)alkoxy((C₀-C₆)alkyl)
each n independently is 1, 2, or 3;
Z is CH, or N;
X is CH or N;
R¹ is H, (C₁-C₆)alkyl, cyano, —(C₁-C₆)OH, halogen, or (C₁-C₆)haloalkyl; and
R² is H, (C₁-C₆)alkyl, cyano, —(C₁-C₆)OH, halogen, or (C₁-C₆)haloalkyl.

2. A compound of claim 1 or a pharmaceutically acceptable salt thereof, wherein R¹ is hydrogen, (C₁-C₆)alkyl, cyano, or halogen.

3. A compound of claim 2 or a pharmaceutically acceptable salt thereof, wherein R¹ is methyl, ethyl, propyl, isopropyl, cyano, hydrogen, chloro, or fluoro.

4. A compound of claim 1 or a pharmaceutically acceptable salt thereof, wherein R¹ is hydrogen, cyano, or chloro.

5. A compound of claim 1 or a pharmaceutically acceptable salt thereof, wherein R² is methyl, ethyl, propyl, isopropyl, cyano, hydrogen, chloro, or fluoro.

6. A compound of claim 1 or a pharmaceutically acceptable salt thereof, wherein R² is methyl, hydrogen, chloro, or fluoro.

7. A compound of claim 1 or a pharmaceutically acceptable salt thereof, wherein X is CH.

8. A compound of claim 1 or a pharmaceutically acceptable salt thereof, wherein X is N.

9. A compound of claim 1 or a pharmaceutically acceptable salt thereof, wherein W is CR^{a}R^{b}.

10. A compound of claim 1 or a pharmaceutically acceptable salt thereof, wherein W is NR^{a}.

11. A compound of claim 1 or a pharmaceutically acceptable salt thereof, wherein R^{b} is hydrogen, methyl, ethyl, propyl, tert-butyl or isopropyl.

12. A compound of claim 1 or a pharmaceutically acceptable salt thereof, wherein R^{b} is hydrogen, or methyl.

13. A compound of claim 1 or a pharmaceutically acceptable salt thereof, wherein R^{a} is selected from methylcarbonyl, ethylcarbonyl, hydrogen, cyano, difluoromethyl, 2,2,2,-trifluoroethyl, fluoromethyl, fluoro, chloro, oxo, methyl, ethyl, propyl, isopropyl, butyl, methoxy, ethoxy, methylsulfonyl, ethylsulfonyl, hydroxymethyl, hydroxyethyl, hydroxy, amino, and trifluoromethyl.

14. A compound of claim 1 or a pharmaceutically acceptable salt thereof, wherein R^{a} is selected from methylcarbonyl, hydrogen, cyano, difluoromethyl, fluoro, chloro, oxo, methyl, methoxy, methylsulfonyl, hydroxy, amino, and trifluoromethyl.

15. A compound of claim 1 or a pharmaceutically acceptable salt thereof, selected from:
6,7-dichloro-3-(2-methoxyethyl)-4,9-dihydro-1H-pyrrolo[3,2-h][2,1,3]benzothiadiazine 2,2-dioxide;
6-chloro-3-(2-methoxyethyl)-4,9-dihydro-1H-pyrrolo[3,2-h][2,1,3]benzothiadiazine 2,2-dioxide;
6,7-dichloro-1,3,4,9-tetrahydropyrrolo[3,2-h][2,1,3]benzothiadiazine 2,2-dioxide,
6,7-dichloro-3-cyclohexyl-4,9-dihydro-1H-pyrrolo[3,2-h][2,1,3]benzothiadiazine 2,2-dioxide;
6,7-dichloro-1,3,4,9-tetrahydropyrrolo[3,2-h][2,1]benzothiazine 2,2-dioxide;
3-benzyl-6,7-dichloro-4,9-dihydro-1H-pyrrolo[3,2-h][2,1,3]benzothiadiazine 2,2-dioxide;
6,7-dichloro-3-phenyl-4,9-dihydro-1H-pyrrolo[3,2-h][2,1,3]benzothiadiazine 2,2-dioxide;
6-chloro-3-(2-methoxyethyl)-2,2-dioxo-4,9-dihydro-1H-pyrrolo[3,2-h][2,1,3]benzothiadiazine-7-carbonitrile;
7-chloro-3-propyl-1,9-dihydropyrrolo[3,2-h][2,1]benzothiazine 2,2-dioxide;
7-chloro-3-propyl-1,3,4,9-tetrahydropyrrolo[3,2-h][2,1]benzothiazine 2,2-dioxide;
N-[(7-amino-3,4-dichloro-1H-indol-6-yl)methyl]-N-[(3-chlorophenyl)methyl]methanesulfonamide;
6,7-dichloro-3-[(6-chloro-3-pyridyl)methyl]-4,9-dihydro-1H-pyrrolo[3,2-h][2,1,3]benzothiadiazine 2,2-dioxide;
6,7-dichloro-3-[(4-chlorophenyl)methyl]-4,9-dihydro-1H-pyrrolo[3,2-h][2,1,3]benzothiadiazine 2,2-dioxide;
6,7-dichloro-3-[(2-chlorophenyl)methyl]-4,9-dihydro-1H-pyrrolo[3,2-h][2,1,3]benzothiadiazine 2,2-dioxide;
6,7-dichloro-3-methyl-4,9-dihydro-1H-pyrrolo[3,2-h][2,1,3]benzothiadiazine 2,2-dioxide;
3-benzyl-6,7-dichloro-1,3,4,9-tetrahydropyrrolo[3,2-h][2,1]benzothiazine 2,2-dioxide;
5-[(6,7-dichloro-2,2-dioxo-4,9-dihydro-1H-pyrrolo[3,2-h][2,1,3]benzothiadiazin-3-yl)methyl]-1H-pyridin-2-one;
6,7-dichloro-3-(4-fluorobutyl)-4,9-dihydro-1H-pyrrolo[3,2-h][2,1,3]benzothiadiazine 2,2-dioxide;
6,7-dichloro-3-(2-pyridylmethyl)-4,9-dihydro-1H-pyrrolo[3,2-h][2,1,3]benzothiadiazine 2,2-dioxide;
6,7-dichloro-3-(4-pyridylmethyl)-4,9-dihydro-1H-pyrrolo[3,2-h][2,1,3]benzothiadiazine 2,2-dioxide;
6,7-dichloro-3-[(6-methoxy-3-pyridyl)methyl]-4,9-dihydro-1H-pyrrolo[3,2-h][2,1,3]benzothiadiazine 2,2-dioxide;
6,7-dichloro-3-(2-methoxyethyl)-1,3,4,9-tetrahydropyrrolo[3,2-h][2,1]benzothiazine 2,2-dioxide;
6,7-dichloro-3-(1-phenylethyl)-4,9-dihydro-1H-pyrrolo[3,2-h][2,1,3]benzothiadiazine 2,2-dioxide;
2-(6,7-dichloro-2,2-dioxo-4,9-dihydro-1H-pyrrolo[3,2-h][2,1,3]benzothiadiazin-3-yl)ethanol;
6,7-dichloro-3-(4,4,5,5,5-pentafluoropentyl)-4,9-dihydro-1H-pyrrolo[3,2-h][2,1,3]benzothiadiazine 2,2-dioxide;
6,7-dichloro-3-(3-pyridylmethyl)-4,9-dihydro-1H-pyrrolo[3,2-h][2,1,3]benzothiadiazine 2,2-dioxide;
6,7-dichloro-3-[(2-chloro-3-pyridyl)methyl]-4,9-dihydro-1H-pyrrolo[3,2-h][2,1,3]benzothiadiazine 2,2-dioxide;
2-(6,7-dichloro-2,2-dioxo-4,9-dihydro-1H-pyrrolo[3,2-h][2,1,3]benzothiadiazin-3-yl)-N-methyl-ethanamine; 2,2,2-trifluoroacetic acid;
(3R)-6,7-dichloro-3-(2-methoxyethyl)-1,3,4,9-tetrahydropyrrolo[3,2-h][2,1]benzothiazine 2,2-dioxide;
(3S)-6,7-dichloro-3-(2-methoxyethyl)-1,3,4,9-tetrahydropyrrolo[3,2-h][2,1]benzothiazine 2,2-dioxide;
6,7-dichloro-1,3,4,9-tetrahydropyrrolo[1,2]pyrido[3,5-d][1,2,6]thiadiazine 2,2-dioxide;
6,7-dichloro-3-(4,4,4-trifluorobutyl)-4,9-dihydro-1H-pyrrolo[3,2-h][2,1,3]benzothiadiazine 2,2-dioxide;
6,7-dichloro-3-(pyrimidin-5-ylmethyl)-4,9-dihydro-1H-pyrrolo[3,2-h][2,1,3]benzothiadiazine 2,2-dioxide;
6,7-dichloro-3-(oxetan-3-ylmethyl)-4,9-dihydro-1H-pyrrolo[3,2-h][2,1,3]benzothiadiazine 2,2-dioxide;
6,7-dichloro-3-[(3-chloro-4-pyridyl)methyl]-4,9-dihydro-1H-pyrrolo[3,2-h][2,1,3]benzothiadiazine 2,2-dioxide;
6,7-dichloro-3-[(5-methyl-1,3,4-oxadiazol-2-yl)methyl]-4,9-dihydro-1H-pyrrolo[3,2-h][2,1,3]benzothiadiazine 2,2-dioxide;
6,7-dichloro-3-[(1-methyl-1,2,4-triazol-3-yl)methyl]-4,9-dihydro-1H-pyrrolo[3,2-h][2,1,3]benzothiadiazine 2,2-dioxide;
6,7-dichloro-3-[(2-methyl-1,2,4-triazol-3-yl)methyl]-4,9-dihydro-1H-pyrrolo[3,2-h][2,1,3]benzothiadiazine 2,2-dioxide;
6,7-dichloro-3-(4-piperidylmethyl)-4,9-dihydro-1H-pyrrolo[3,2-h][2,1,3]benzothiadiazine 2,2-dioxide;
N-[2-(6,7-dichloro-2,2-dioxo-4,9-dihydro-1H-pyrrolo[3,2-h][2,1,3]benzothiadiazin-3-yl)ethyl]acetamide;
2-(6,7-dichloro-2,2-dioxo-4,9-dihydro-1H-pyrrolo[3,2-h][2,1,3]benzothiadiazin-3-yl)-N,N-dimethyl-ethanamine;
6,7-dichloro-3-(pyridazin-3-ylmethyl)-4,9-dihydro-1H-pyrrolo[3,2-h][2,1,3]benzothiadiazine 2,2-dioxide;
6,7-dichloro-3-[(1-methylpyrazol-4-yl)methyl]-4,9-dihydro-1H-pyrrolo[3,2-h][2,1,3]benzothiadiazine 2,2-dioxide;
6,7-dichloro-3-(tetrahydropyran-4-ylmethyl)-4,9-dihydro-1H-pyrrolo[3,2-h][2,1,3]benzothiadiazine 2,2-dioxide;
1-[4-[(6,7-dichloro-2,2-dioxo-4,9-dihydro-1H-pyrrolo[3,2-h][2,1,3]benzothiadiazin-3-yl)methyl]-1-piperidyl]ethenone;
2-[(6,7-dichloro-2,2-dioxo-4,9-dihydro-1H-pyrrolo[3,2-h][2,1,3]benzothiadiazin-3-yl)methyl]benzonitrile;
1-[3-[(6,7-dichloro-2,2-dioxo-4,9-dihydro-1H-pyrrolo[3,2-h][2,1,3]benzothiadiazin-3-yl)methyl]azetidin-1-yl]ethenone;
6,7-dichloro-3-[(6-methyl-3-pyridyl)methyl]-4,9-dihydro-1H-pyrrolo[3,2-h][2,1,3]benzothiadiazine 2,2-dioxide;
6,7-dichloro-3-[[6-(trifluoromethyl)-3-pyridyl]methyl]-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]benzonitrile;
3-[(6,7-dichloro-2,2-dioxo-4,9-dihydro-1H-pyrrolo[3,2-h][2,1,3]benzothiadiazin-3-yl)methyl]benzonitrile;
6,7-dichloro-3-(pyrimidin-4-ylmethyl)-4,9-dihydro-1H-pyrrolo[3,2-h][2,1,3]benzothiadiazine 2,2-dioxide;
6,7-dichloro-3-[[6-(difluoromethyl)-3-pyridyl]methyl]-4,9-dihydro-1H-pyrrolo[3,2-h][2,1,3]benzothiadiazine 2,2-dioxide;
6,7-dichloro-3-[(4-chloro-3-pyridyl)methyl]-4,9-dihydro-1H-pyrrolo[3,2-h][2,1,3]benzothiadiazine 2,2-dioxide;
6,7-dichloro-3-[(4-methyl-3-pyridyl)methyl]-4,9-dihydro-1H-pyrrolo[3,2-h][2,1,3]benzothiadiazine 2,2-dioxide;
6,7-dichloro-3-((5-chloropyridin-3-yl)methyl)-1,3,4,9-tetrahydro-[1,2,6]thiadiazino[4,3-g]indole 2,2-dioxide;
6-[(6,7-dichloro-2,2-dioxo-4,9-dihydro-1H-pyrrolo[3,2-h][2,1,3]benzothiadiazin-3-yl)methyl]-1H-pyridin-2-one;
6-chloro-2,2-dioxo-1,3,4,9-tetrahydropyrrolo[3,2-h][2,1,3]benzothiadiazine-7-carbonitrile;
6,7-dichloro-3-(pyrazin-2-ylmethyl)-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]-1H-pyridin-2-one;
4-[(6,7-dichloro-2,2-dioxo-4,9-dihydro-1H-pyrrolo[3,2-h][2,1,3]benzothiadiazin-3-yl)methyl]-1-methyl-pyridin-2-one;
6-[(6,7-dichloro-2,2-dioxo-4,9-dihydro-1H-pyrrolo[3,2-h][2,1,3]benzothiadiazin-3-yl)methyl]-1-methyl-pyridin-2-one;
6,7-dichloro-3-[(2-chloro-4-pyridyl)methyl]-4,9-dihydro-1H-pyrrolo[3,2-h][2,1,3]benzothiadiazine 2,2-dioxide;
6,7-dichloro-3-(2-methoxyethyl)-4,9-dihydro-1H-pyrazolo[4,3-h][2,1,3]benzothiadiazine 2,2-dioxide;
5-[(6,7-dichloro-2,2-dioxo-4,9-dihydro-1H-pyrrolo[3,2-h][2,1,3]benzothiadiazin-3-yl)methyl]-1-methyl-pyridin-2-one;
6,7-dichloro-3,3-dimethyl-4,9-dihydro-1H-pyrrolo[3,2-h][2,1]benzothiazine 2,2-dioxide;
6,7-dichloro-3-(pyridazin-4-ylmethyl)-4,9-dihydro-1H-pyrrolo[3,2-h][2,1,3]benzothiadiazine 2,2-dioxide;
6,7-dichloro-3-(3-methoxypropyl)-4,9-dihydro-1H-pyrrolo[3,2-h][2,1,3]benzothiadiazine 2,2-dioxide;
6,7-dichloro-3-[(4-fluorophenyl)methyl]-4,9-dihydro-1H-pyrrolo[3,2-h][2,1,3]benzothiadiazine 2,2-dioxide;
6,7-dichloro-3-[(2-fluorophenyl)methyl]-4,9-dihydro-1H-pyrrolo[3,2-h][2,1,3]benzothiadiazine 2,2-dioxide;
6,7-dichloro-3-(2-morpholinoethyl)-4,9-dihydro-1H-pyrrolo[3,2-h][2,1,3]benzothiadiazine 2,2-dioxide;
6,7-dichloro-3-[(1-methylsulfonyl-4-piperidyl)methyl]-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]phenol;
2-[(6,7-dichloro-2,2-dioxo-4,9-dihydro-1H-pyrrolo[3,2-h][2,1,3]benzothiadiazin-3-yl)methyl]aniline;
6,7-dichloro-3-[(3-fluorophenyl)methyl]-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]aniline;
6,7-dichloro-3-(3-methylsulfonylpropyl)-4,9-dihydro-1H-pyrrolo[3,2-h][2,1,3]benzothiadiazine 2,2-dioxide;
4-[(6,7-dichloro-2,2-dioxo-4,9-dihydro-1H-pyrrolo[3,2-h][2,1,3]benzothiadiazin-3-yl)methyl]aniline;
7-chloro-6-fluoro-3-(2-methoxyethyl)-4,9-dihydro-1H-pyrrolo[3,2-h][2,1,3]benzothiadiazine 2,2-dioxide;
7-chloro-3-tetrahydropyran-3-yl-4,9-dihydro-1H-pyrrolo[3,2-h][2,1,3]benzothiadiazine 2,2-dioxide;
7-chloro-3-(2-methoxyethyl)-4,9-dihydro-1H-pyrrolo[3,2-h][2,1,3]benzothiadiazine 2,2-dioxide;
7-chloro-3-(3-pyridyl)-4,9-dihydro-1H-pyrrolo[3,2-h][2,1,3]benzothiadiazine 2,2-dioxide;
6,7-dichloro-3-[(1-methyl-4-piperidyl)methyl]-4,9-dihydro-1H-pyrrolo[3,2-h][2,1,3]benzothiadiazine 2,2-dioxide;
6,7-dichloro-3-(2-phenylethyl)-4,9-dihydro-1H-pyrrolo[3,2-h][2,1,3]benzothiadiazine 2,2-dioxide;
6,7-dichloro-3-[(3,3-difluorocyclobutyl)methyl]-4,9-dihydro-1H-pyrrolo[3,2-h][2,1,3]benzothiadiazine 2,2-dioxide;
6,7-dichloro-3-(tetrahydrofuran-3-ylmethyl)-4,9-dihydro-1H-pyrrolo[3,2-h][2,1,3]benzothiadiazine 2,2-dioxide;
7-chloro-3-(2-methoxyethyl)-6-methyl-4,9-dihydro-1H-pyrrolo[3,2-h][2,1,3]benzothiadiazine 2,2-dioxide;
3-[(6,7-dichloro-2,2-dioxo-4,9-dihydro-1H-pyrrolo[3,2-h][2,1,3]benzothiadiazin-3-yl)methyl]cyclobutanol;
6,7-dichloro-3-(tetrahydrofuran-2-ylmethyl)-4,9-dihydro-1H-pyrrolo[3,2-h][2,1,3]benzothiadiazine 2,2-dioxide;
6,7-dichloro-3-(3-piperidylmethyl)-4,9-dihydro-1H-pyrrolo[3,2-h][2,1,3]benzothiadiazine 2,2-dioxide;
1-[3-[(6,7-dichloro-2,2-dioxo-4,9-dihydro-1H-pyrrolo[3,2-h][2,1,3]benzothiadiazin-3-yl)methyl]-1-piperidyl]ethenone;
2-(6,7-dichloro-2,2-dioxo-4,9-dihydro-1H-pyrrolo[3,2-h][2,1,3]benzothiadiazin-3-yl)-1-morpholino-ethanone;
3-allyl-6,7-dichloro-4,9-dihydro-1H-pyrrolo[3,2-h][2,1,3]benzothiadiazine 2,2-dioxide;
6,7-dichloro-3-[2-(oxetan-3-yl)ethyl]-4,9-dihydro-1H-pyrrolo[3,2-h][2,1,3]benzothiadiazine 2,2-dioxide;
6,7-dichloro-3-(tetrahydropyran-3-ylmethyl)-4,9-dihydro-1H-pyrrolo[3,2-h][2,1,3]benzothiadiazine 2,2-dioxide;
6,7-dichloro-3-(tetrahydropyran-2-ylmethyl)-4,9-dihydro-1H-pyrrolo[3,2-h][2,1,3]benzothiadiazine 2,2-dioxide;
3-chloro-4-[(6,7-dichloro-2,2-dioxo-4,9-dihydro-1H-pyrrolo[3,2-h][2,1,3]benzothiadiazin-3-yl)methyl]-1H-pyridin-2-one;
5-chloro-4-[(6,7-dichloro-2,2-dioxo-4,9-dihydro-1H-pyrrolo[3,2-h][2,1,3]benzothiadiazin-3-yl)methyl]-1H-pyridin-2-one;
6-fluoro-3-(2-methoxyethyl)-2,2-dioxo-4,9-dihydro-1H-pyrrolo[3,2-h][2,1,3]benzothiadiazine-7-carbonitrile; and
3-(2-methoxyethyl)-2,2-dioxo-4,9-dihydro-1H-pyrrolo[3,2-h][2,1,3]benzothiadiazine-7-carbonitrile.

16. A pharmaceutical composition comprising a compound of claim 1 or a pharmaceutically acceptable salt thereof, and a pharmaceutically acceptable diluent or carrier.

17. (canceled)

18. (canceled)

19. A method of treating cancer in a subject in need thereof, comprising administering to said subject a therapeutically effective amount of a compound of any one of claim 1 or a pharmaceutically acceptable salt thereof.

20. A method of modulating at least one activity selected from RBM39 activity and DCAF 15 activity, in a patient in need thereof, comprising administering to said subject a therapeutically effective amount compound of claim 1 or a pharmaceutically acceptable salt thereof.

21. A method of modulating at least one activity of claim 1, or a pharmaceutically acceptable salt thereof, wherein the at least one activity is RBM39.

22. A method of modulating at least one activity of claim 1, or a pharmaceutically acceptable salt thereof, wherein the at least one activity is DCAF 15 activity.
